(19) United States
(12) Patent Application Publication
(10) Pub. No.: US 2019/0112590 A1
(43) Pub. Date: Apr. 18, 2019

(54) ARABINOSE ISOMERASES FOR YEAST

(71) Applicant: EI DU PONT DE NEMOURS AND COMPANY, Wilmington, DE (US)

(72) Inventors: Zhongqiang Chen, Wilmington, DE (US); Min Qi, Hockessin, DE (US); Jianjun Yang, Hockessin, DE (US); Barbara RICE, Wilmington, DE (US)

(21) Appl. No.: 16/091,175

(22) PCT Filed: Apr. 5, 2017
(86) PCT No.: PCT/US2017/026133

§ 371 Date: Oct. 4, 2018

Related U.S. Application Data
(60) Provisional application No. 62/319,945, filed on Apr. 8, 2016.

(51) Int. Cl. C12N 9/90; C12N 15/52; C12P 7/18; C12P 7/16; C12P 7/06

(52) U.S. Cl. CPC C12N 9/90; C12N 15/52; C12Y 503/01004; C12P 7/16; C12P 7/06; C12P 7/18

(57) ABSTRACT

A group of arabinose isomerases are disclosed that provide effective amounts of activity for use of arabinose in production of ethanol, when expressed in yeast cells expressing the other enzymes of an arabinose utilization pathway. The group of arabinose isomerases represents a clade of a phylogenetic tree, having a distinguishing conserved amino acid sequence motif. Other useful arabinose isomerases are also disclosed.

This application claims the benefit of U.S. Provisional Application No. 62/319,945 (filed Apr. 8, 2016), which is incorporated herein by reference in its entirety.

### FIELD OF INVENTION

The field of invention relates to genetic engineering, and more specifically to engineering yeast to enhance utilization of arabinose during fermentation.

### REFERENCE TO SEQUENCE LISTING SUBMITTED ELECTRONICALLY

The official copy of the sequence listing is submitted electronically via EFS-Web as an ASCII formatted sequence listing with a file named CL6357WOPCT_SequenceListing_ST25_ExtraLinesRemoved created on Mar. 31, 2017, and having a size of 284 kilobytes and is filed concurrently with the specification. The sequence listing contained in this ASCII-formatted document is part of the specification and is herein incorporated by reference in its entirety.

### BACKGROUND

Currently, fermentative production of ethanol is typically done with yeast, particularly Saccharomyces cerevisiae, using hexoses obtained from grains or mash as the carbohydrate source. Use of hydrolysate prepared from cellulosic biomass as a carbohydrate source for fermentation is desirable, as this is a readily renewable resource that does not compete with the food supply. The most abundant sugar in cellulosic biomass hydrolysate is glucose, while the pentoses xylose and arabinose are also present. Many biocatalysts, including the yeast Saccharomyces cerevisiae, are not naturally capable of metabolizing xylose or arabinose, but can be engineered to express xylose and/or arabinose utilization pathways. One approach to engineering a xylose utilization pathway in yeast includes introduction of xylose isomerase, and increasing expression of the pentose phosphate pathway including xylulokinase, transaldolase, transketolase 1, D-ribulose-5-phosphate 3-epimerase, and ribose 5-phosphate ketol-isomerase. Use of arabinose can be achieved by additionally engineering expression of L-arabinose isomerase, L-ribulokinase, and L-ribulose-5-phosphate 4-epimerase (e.g., Becker and Boles, Appl. Environ. Microbiol. 69:4144-4150).

Though yeast strains engineered in this manner can utilize arabinose, such arabinose use is typically inefficient due to poor efficiency of arabinose isomerase, which operates at the first step of the bacterial-type arabinose assimilation pathway. Thus, there remains a need for arabinose isomerases that are more effective when expressed in yeast, and engineered yeast cells that express an arabinose isomerase that allows greater efficiency of arabinose utilization.

### SUMMARY

In one embodiment, the present disclosure concerns a recombinant yeast cell comprising an arabinose utilization pathway that comprises a polypeptide having arabinose isomerase activity, wherein the yeast cell comprises a heterologous polynucleotide encoding the polypeptide, wherein the polypeptide comprises a motif that is at least 90% identical to SEQ ID NO:67, and wherein the position of the motif in the polypeptide corresponds with positions 237-269 of SEQ ID NO:7.

In another embodiment, the present disclosure concerns a method for producing a yeast cell having arabinose isomerase activity. This method comprises: (a) providing a yeast cell lacking arabinose isomerase; and (b) introducing a heterologous polynucleotide into the yeast cell, wherein the heterologous polynucleotide encodes a polypeptide having arabinose isomerase activity, and wherein the polypeptide comprises a motif that is at least 90% identical to SEQ ID NO:67.

In another embodiment, the present disclosure concerns a method of producing a target compound from arabinose comprising: (a) providing a recombinant yeast cell as disclosed herein; (b) growing the yeast cell of (a) in medium comprising arabinose, wherein the target compound is produced; and (c) optionally isolating the target compound of (b).

In another embodiment, the present disclosure concerns a recombinant yeast cell comprising an arabinose utilization pathway that comprises a polypeptide having arabinose isomerase activity, wherein the yeast cell comprises a heterologous polynucleotide encoding the polypeptide, and wherein the polypeptide comprises an amino acid sequence that is at least 85% identical to SEQ ID NO:15 or 20. The present disclosure also concerns a method of using such a yeast cell to produce a target compound from arabinose comprising: (a) providing the yeast cell; (b) growing the yeast cell of (a) in medium comprising arabinose, wherein the target compound is produced; and c) optionally isolating the target compound of (b).

### BRIEF DESCRIPTION OF THE DRAWINGS AND SEQUENCES

FIG. 1 shows a phylogenetic tree of twenty candidate arabinose isomerase proteins, and the B. subtilis arabinose isomerase protein.

FIG. 2A shows the 237-269 Motif formula I (SEQ ID NO:67).

FIG. 2B shows the 237-269 Motif formula II (SEQ ID NO:67) with bold and underline highlighting of specific positions, as described in the detailed description.

FIG. 2C shows an alignment of twenty candidate arabinose isomerases and the B. subtilis arabinose isomerase in the region of positions 237-269 (with reference to SEQ ID NO:7), with the six members of the 237-269 Motif clade at the top. The BSaraA amino acid sequence used in this alignment is SEQ ID NO:41.

FIG. 3A is a plasmid map of pSX01 (SEQ ID NO:43).

FIG. 3B is a plasmid map of pSX208 (SEQ ID NO:47).

FIG. 4A is a plasmid map of pSX209 (SEQ ID NO:48).

FIG. 4B is a plasmid map of pSX210 (SEQ ID NO:49).

FIG. 5A is a plasmid map of pSA0-B (SEQ ID NO:58).

FIG. 5B is a plasmid map of pSA503 (SEQ ID NO:59).

FIG. 6 is a graph showing growth (OD600), arabinose use, and production of ethanol during fermentation by yeast strain PX182-araBAD5030 in medium containing arabinose as the only sugar.

FIG. 7 is a graph of in vitro arabinose isomerase activities (μmol/mg/min) from arabinose- and xylose-utilizing yeast strains expressing different candidate arabinose isomerases or the B. subtilis arabinose isomerase.

TABLE 1
SEQ ID NOs for Amino Acid (AA) and Nucleotide
(NT) Sequences (Codon-Optimized Coding Regions)
of Candidate Arabinose Isomerases.

| Designation | SEQ ID NO: AA | SEQ ID NO: NT |
|---|---|---|
| HMPREF9412_4417 | 1 | 21 |
| POTG_01507 | 2 | 22 |
| HMPREF9374_3716 | 3 | 23 |
| DORFOR_01282 | 4 | 24 |
| HMPREF0994_04908 | 5 | 25 |
| NODE_4061684 | 6 | 26 |
| NODE_3664377 | 7 | 27 |
| NODE_458803 | 8 | 28 |
| NODE_3921064 | 9 | 29 |
| NODE_3693095 | 10 | 30 |
| DORLON_00938 | 11 | 31 |
| HMPREF9469_04726 | 12 | 32 |
| HMPREF9467_00216 | 13 | 33 |
| RTO_26010 | 14 | 34 |
| BRYFOR_08166 | 15 | 35 |
| RUMOBE_03031 | 16 | 36 |
| NODE_3658038 | 17 | 37 |
| NODE_4175755 | 18 | 38 |
| NODE_2588280 | 19 | 39 |
| NODE_3735508 | 20 | 40 |

Arabinose isomerase designations in Table 1 are from the cow rumen metagenome dataset (“NODE” designations; Hess et al., Science 331:463-467, incorporated herein by reference) or the human microbiome dataset (The Human Microbiome Jumpstart Reference Strains Consortium et al., Science 328:994-999, incorporated herein by reference).

SEQ ID NO:41 is the amino acid sequence of the arabinose isomerase from B. subtilis (BSaraA).

SEQ ID NO:42 is the nucleotide sequence of the codon-optimized coding to region for the B. subtilis arabinose isomerase.

SEQ ID NO:43 is the nucleotide sequence of the plasmid pSX01.

SEQ ID NO:44 is the amino acid sequence of the xylose isomerase VDxylA.

SEQ ID NO:45 is the nucleotide sequence of the codon-optimized coding region for the VDxylA arabinose isomerase.

SEQ ID NO:46 is the nucleotide sequence of the 2966-bp chimeric expression cassette designated as UAS(FBA1)::PDC1p::VDxylA::ILV5t.

SEQ ID NO:47 is the nucleotide sequence of the plasmid pSX208.

SEQ ID NO:48 is the nucleotide sequence of the plasmid pSX209.

SEQ ID NO:49 is the nucleotide sequence of the plasmid pSX210.

SEQ ID NO:50 is the nucleotide sequence of the CRE recombinase vector pJT254.

SEQ ID NO:51 is the nucleotide sequence of the 2429-bp chimeric expression cassette designated as ADHp::BSaraA::CYC1t.

SEQ ID NO:52 is the amino acid sequence of the E. coli araB gene-encoded ribulokinase.

SEQ ID NO:53 is the nucleotide sequence of the codon-optimized coding region for the E. coli ribulokinase.

SEQ ID NO:54 is the nucleotide sequence of the 2907-bp chimeric expression cassette designated as ILV5p::ECaraB::PHO13-3′UTR.

SEQ ID NO:55 is the amino acid sequence of the E. coli araD gene-encoded L-ribulose-5-phosphate 4-epimerase.

SEQ ID NO:56 is the nucleotide sequence of the codon-optimized coding region for the E. coli L-ribulose-5-phosphate 4-epimerase.

SEQ ID NO:57 is the nucleotide sequence of the 1691-bp chimeric expression cassette designated as GPDp::ECaraD::ADH1t.

SEQ ID NO:58 is the nucleotide sequence of the plasmid pSA0-B.

SEQ ID NO:59 is the nucleotide sequence of the plasmid pSA503.

SEQ ID NO:60 is the amino acid sequence of the arabinose isomerase from E. coli.

SEQ ID NO:61 is the amino acid sequence of the arabinose isomerase from Bacillus licheniformis.

SEQ ID NO:62 is the amino acid sequence of the arabinose isomerase from Clostridium acetobutylicum.

SEQ ID NO:63 is the amino acid sequence of the arabinose isomerase from Leuconostoc mesenteroides.

SEQ ID NO:64 is the amino acid sequence of the arabinose isomerase from Lactobacillus plantarum.

SEQ ID NO:65 is the amino acid sequence of the arabinose isomerase from Pediococcus pentosaceus.

SEQ ID NO:66 is the amino acid sequence representing a “237-269 Motif”.

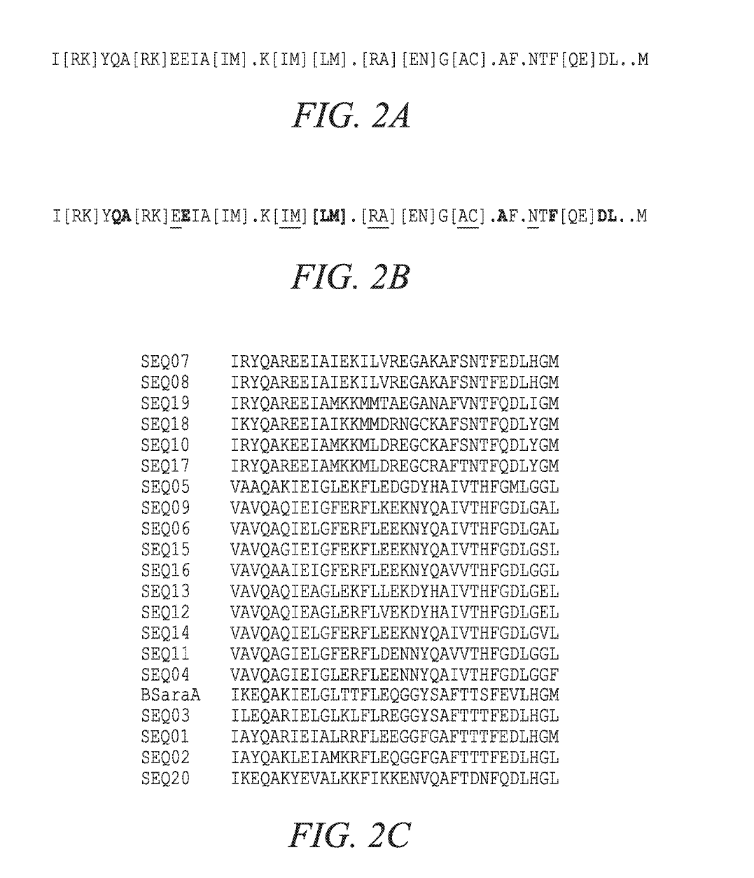

SEQ ID NO:67 is the amino acid sequence of the 237-269 Motif shown in FIG. 2A.

Each of SEQ ID NOs:68-71 is an amino acid sequence that can optionally be excluded in certain embodiments of the present disclosure.

### DETAILED DESCRIPTION

The disclosures of all cited patent and non-patent literature are incorporated herein by reference in their entirety.

Unless otherwise disclosed, the terms “a” and “an” as used herein are intended to encompass one or more (i.e., at least one) of a stated feature.

Where present, all ranges are inclusive and combinable, except as otherwise noted. For example, when a range of “1 to 5” is recited, the recited range should be construed as including ranges “1 to 4”, “1 to 3”, “1-2”, “1-2 & 4-5”, “1-3 & 5”, and the like.

The terms “about”, “approximately” and the like in some aspects, as used to modify certain numerical values herein, refer to being within 5%-10% of the stated numerical value.

The terms “arabinose isomerase”, “L-arabinose isomerase” and the like refer to an enzyme that catalyzes the conversion of L-arabinose to L-ribulose. Arabinose isomerases belong to the group of enzymes classified in Enzyme Commission (EC) entry 5.3.1.4.

The terms “carbon substrate”, “fermentable carbon substrate” and the like refer to a carbon source capable of being metabolized by microorganisms. A type of carbon substrate is “fermentable sugars” which refer to oligosaccharides and monosaccharides that can be used as a carbon source by a microorganism in a fermentation process. Arabinose and xylose are examples of fermentable sugars.

The term “lignocellulosic” refers to a composition comprising both lignin and cellulose. Lignocellulosic material may also comprise hemicellulose.

The term “cellulosic” refers to a composition comprising cellulose and additional components, which may include hemicellulose and lignin.

The term “saccharification” refers to the production of fermentable sugars from polysaccharides such as cellulose and hemicellulose. Saccharification can be done via chemical and/or enzymatic means.

“Biomass” refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Further examples of biomass include, but are not limited to, corn cobs, crop residues such as corn husks, corn stover, corn grain fiber, grasses, beet pulp, wheat straw, wheat chaff, oat straw, barley straw, barley hulls, hay, rice straw, rice hulls, switchgrass, miscanthus, cord grass, reed canary grass, waste paper, sugar cane bagasse, sorghum bagasse, sorghum stover, soybean stover, components obtained from milling of grains, trees, branches, roots, leaves, wood chips, sawdust, palm waste, shrubs and bushes, vegetables, fruits, flowers, and animal manure.

The term “pretreated biomass” refers to biomass that has been subjected to thermal, physical and/or chemical treatment to increase the availability of polysaccharides in the biomass to saccharification enzymes. Biomass pretreatment is typically done before saccharification.

“Biomass hydrolysate” refers to the product resulting from saccharification of biomass, and comprises fermentable sugars.

The terms “target compound”, “target chemical” and the like refer to a compound made by a microorganism via an endogenous or recombinant biosynthetic/metabolic pathway that is able to metabolize a fermentable carbon source to produce the target compound.

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “polynucleotide”, “polynucleotide sequence”, “nucleic acid molecule” and the like are used interchangeably herein. These terms encompass nucleotide sequences and the like. A polynucleotide may be a polymer of DNA or RNA that is single- or double-stranded, that optionally contains synthetic, non-natural or altered nucleotide bases. A polynucleotide may be comprised of one or more segments of cDNA, genomic DNA, synthetic DNA, or mixtures thereof. Nucleotides (ribonucleotides or deoxyribonucleotides) can be referred to by a single letter designation as follows: “A” for adenylate or deoxyadenylate (for RNA or DNA, respectively), “C” for cytidylate or deoxycytidylate (for RNA or DNA, respectively), “G” for guanylate or deoxyguanylate (for RNA or DNA, respectively), “U” for uridylate (for RNA), “T” for deoxythymidylate (for DNA), “R” for purines (A or G), “Y” for pyrimidines (C or T), “K” for G or T, “H” for A or C or T, “I” for inosine, “W” for A or T, and “N” for any nucleotide (e.g., N can be A, C, T, or G, if referring to a DNA sequence; N can be A, C, U, or G, if referring to an RNA sequence).

The terms “motif”, “conserved motif” and the like herein refer to a distinctive and recurring structural unit, such as within an amino acid sequence. By “recurring” it is meant that a motif occurs in multiple related polypeptides, for example.

Herein, a first polynucleotide sequence that is “complementary” to a second polynucleotide sequence can alternatively be referred to as being in the “antisense” orientation with the second sequence.

The term “gene” as used herein refers to a DNA polynucleotide sequence that expresses an RNA (RNA is transcribed from the DNA polynucleotide sequence) from a coding region, which RNA can be a messenger RNA (encoding a protein) or a non-protein-coding RNA. A gene may refer to the coding region alone, or may include regulatory sequences upstream and/or downstream to the coding region (e.g., promoters, 5′-untranslated regions, 3′-transcription terminator regions). A coding region encoding a protein can alternatively be referred to herein as an “open reading frame” (ORF). A gene that is “native” or “endogenous” refers to a gene as found in nature with its own regulatory sequences; such a gene is located in its natural location in the genome of a host cell. A “chimeric” gene refers to any gene that is not a native gene, comprising regulatory and coding sequences that are not found together in nature (i.e., the regulatory and coding regions are heterologous with each other). Accordingly, a chimeric gene may comprise regulatory sequences and coding sequences that are derived from different sources, or regulatory sequences and coding sequences derived from the same source, but arranged in a manner different than that found in nature. A “foreign” or “heterologous” gene can refer to a gene that is introduced into the host organism by gene transfer. Foreign/heterologous genes can comprise native genes inserted into a non-native organism, native genes introduced into a new location within the native host, or chimeric genes. The polynucleotide sequences in certain embodiments disclosed herein are heterologous. A “transgene” is a gene that has been introduced into the genome by a gene delivery procedure (e.g., transformation). A “codon-optimized” open reading frame has its frequency of codon usage designed to mimic the frequency of preferred codon usage of the host cell.

The term “heterologous” means not naturally found in the location of interest. For example, a heterologous gene can be one that is not naturally found in a host organism, but that is introduced into the host organism by gene transfer. As another example, a nucleic acid molecule that is present in a chimeric gene can be characterized as being heterologous, as such a nucleic acid molecule is not naturally associated with the other segments of the chimeric gene (e.g., a promoter can be heterologous to a coding sequence).

A “non-native” amino acid sequence or polynucleotide sequence comprised in a cell or organism herein does not occur in a native (natural) counterpart of such cell or organism. Such an amino acid sequence or polynucleotide sequence can also be referred to as being heterologous to the cell or organism.

“Regulatory sequences” as used herein refer to nucleotide sequences located upstream of a gene's transcription start site (e.g., promoter), 5′ untranslated regions, introns, and 3′ non-coding regions, and which may influence the transcription, processing or stability, and/or translation of an RNA transcribed from the gene. Regulatory sequences herein may include promoters, enhancers, silencers, 5′ untranslated leader sequences, introns, polyadenylation recognition sequences, RNA processing sites, effector binding sites, stem-loop structures, and other elements involved in regulation of gene expression. One or more regulatory elements herein may be heterologous to a coding region herein.

A “promoter” as used herein refers to a DNA sequence capable of controlling the transcription of RNA from a gene. In general, a promoter sequence is upstream of the transcription start site of a gene. Promoters may be derived in their entirety from a native gene, or be composed of different elements derived from different promoters found in nature, or even comprise synthetic DNA segments. Promoters that cause a gene to be expressed in a cell at most times under all circumstances are commonly referred to as “constitutive promoters”. One or more promoters herein may be heterologous to a coding region herein.

A “strong promoter” as used herein refers to a promoter that can direct a relatively large number of productive initiations per unit time, and/or is a promoter driving a higher level of gene transcription than the average transcription level of the genes in a cell.

The terms “3′ non-coding sequence”, “transcription terminator” and “terminator” as used herein refer to DNA sequences located downstream of a coding sequence. This includes polyadenylation recognition sequences and other sequences encoding regulatory signals capable of affecting mRNA processing or gene expression.

The terms “upstream” and “downstream” as used herein with respect to polynucleotides refer to “5′ of” and “3′ of”, respectively.

The term “expression” as used herein refers to (i) transcription of RNA (e.g., mRNA or a non-protein-coding RNA) from a coding region, and/or (ii) translation of a polypeptide from mRNA. Expression of a coding region of a polynucleotide sequence can be up-regulated or down-regulated in certain embodiments.

The term “operably linked” as used herein refers to the association of two or more nucleic acid sequences such that the function of one is affected by the other. For example, a promoter is operably linked with a coding sequence when it is capable of affecting the expression of that coding sequence. That is, the coding sequence is under the transcriptional control of the promoter. A coding sequence can be operably linked to one (e.g., promoter) or more (e.g., promoter and terminator) regulatory sequences, for example.

The term “recombinant” when used herein to characterize a DNA sequence such as a plasmid, vector, or construct refers to an artificial combination of two otherwise separated segments of sequence, e.g., by chemical synthesis and/or by manipulation of isolated segments of nucleic acids by genetic engineering techniques.

The term “transformation” as used herein refers to the transfer of a nucleic acid molecule into a host organism or host cell by any method. A nucleic acid molecule that has been transformed into an organism/cell may be one that replicates autonomously in the organism/cell, or that integrates into the genome of the organism/cell, or that exists transiently in the cell without replicating or integrating. Non-limiting examples of nucleic acid molecules suitable for transformation are disclosed herein, such as plasmids and linear DNA molecules. Host organisms/cells herein containing a transforming nucleic acid sequence can be referred to as “transgenic”, “recombinant”, “transformed”, “engineered”, as a “transformant”, and/or as being “modified for exogenous gene expression”, for example.

The terms “control cell” and “suitable control cell” are used interchangeably herein and may be referenced with respect to a cell in which a particular modification (e.g., over-expression of a polynucleotide, down-regulation of a polynucleotide) has been made (i.e., an “experimental cell”). A control cell may be any cell that does not have or does not express the particular modification of the experimental cell. Thus, a control cell may be an untransformed wild type cell or may be genetically transformed but does not express the particular modification. For example, a control cell may be a direct parent of the experimental cell, which direct parent cell does not have the particular modification that is in the experimental cell. Alternatively, a control cell may be a parent of the experimental cell that is removed by one or more generations. Alternatively still, a control cell may be a sibling of the experimental cell, which sibling does not comprise the particular modification that is present in the experimental cell. A control cell can optionally be characterized as a cell as it existed before being modified to be an experimental cell.

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. It would be understood that, when calculating sequence identity between a DNA sequence and an RNA sequence, T residues of the DNA sequence align with, and can be considered “identical” with, U residues of the RNA sequence. For purposes of determining “percent complementarity” of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

Percent identity can be readily determined by any known method, including but not limited to those described in: 1) Computational Molecular Biology (Lesk, A. M., Ed.) Oxford University: NY (1988); 2) Biocomputing: Informatics and Genome Projects (Smith, D. W., Ed.) Academic: NY (1993); 3) Computer Analysis of Sequence Data, Part I (Griffin, A. M., and Griffin, H. G., Eds.) Humana: NJ (1994); 4) Sequence Analysis in Molecular Biology (von Heinje, G., Ed.) Academic (1987); and 5) Sequence Analysis Primer (Gribskov, M. and Devereux, J., Eds.) Stockton: NY (1991), all of which are incorporated herein by reference.

Preferred methods for determining percent identity are designed to give the best match between the sequences tested. Methods of determining identity and similarity are codified in publicly available computer programs, for example. Sequence alignments and percent identity calculations can be performed using the MEGALIGN program of the LASERGENE bioinformatics computing suite (DNASTAR Inc., Madison, Wis.), for example. Multiple alignment of sequences can be performed, for example, using the Clustal method of alignment which encompasses several varieties of the algorithm including the Clustal V method of alignment (described by Higgins and Sharp, CABIOS. 5:151-153 (1989); Higgins, D. G. et al., Comput. Appl. Biosci., 8:189-191 (1992)) and found in the MEGALIGN v8.0 program of the LASERGENE bioinformatics computing suite (DNASTAR Inc.). For multiple alignments, the default values can correspond to GAP PENALTY=10 and GAP LENGTH PENALTY=10. Default parameters for pairwise alignments and calculation of percent identity of protein sequences using the Clustal method can be KTUPLE=1, GAP PENALTY=3, WINDOW=5 and DIAGONALS SAVED=5. For nucleic acids, these parameters can be KTUPLE=2, GAP PENALTY=5, WINDOW=4 and DIAGONALS SAVED=4. Additionally the Clustal W method of alignment can be used (described by Higgins and Sharp, CABIOS. 5:151-153 (1989); Higgins, D. G. et al., Comput. Appl. Biosci. 8:189-191(1992); Thompson, J. D. et al, Nucleic Acids Research, 22 (22): 4673-4680, 1994) and found in the MEGALIGN v8.0 program of the LASERGENE bioinformatics computing suite (DNASTAR Inc.). Default parameters for multiple alignment (protein/nucleic acid) can be: GAP PENALTY=10/15, GAP LENGTH PENALTY=0.2/6.66, Delay Divergen Seqs(%)=30/30, DNA Transition Weight=0.5, Protein Weight Matrix=Gonnet Series, DNA Weight Matrix=IUB.

Various polypeptide amino acid sequences and polynucleotide sequences are disclosed herein as features of certain embodiments. Variants of these sequences that are at least about 70-85%, 85-90%, or 90%-95% identical to the sequences disclosed herein can be used or referenced. Alternatively, a variant amino acid sequence or polynucleotide sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with a sequence disclosed herein. The variant amino acid sequence or polynucleotide sequence has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, or 99% of the function/activity of the disclosed sequence. Any polypeptide amino acid sequence disclosed herein not beginning with a methionine can typically further comprise at least a start-methionine at the N-terminus of the amino acid sequence.

All the amino acid residues at each amino acid position of the proteins disclosed herein are examples. Given that certain amino acids share similar structural and/or charge features with each other (i.e., conserved), the amino acid at each position of a protein herein can be as provided in the disclosed sequences or substituted with a conserved amino acid residue (“conservative amino acid substitution”) as follows:

1. The following small aliphatic, nonpolar or slightly polar residues can substitute for each other: Ala (A), Ser (S), Thr (T), Pro (P), Gly (G);

2. The following polar, negatively charged residues and their amides can substitute for each other: Asp (D), Asn (N), Glu (E), Gln (Q);

3. The following polar, positively charged residues can substitute for each other: His (H), Arg (R), Lys (K);

4. The following aliphatic, nonpolar residues can substitute for each other: Ala (A), Leu (L), Ile (I), Val (V), Cys (C), Met (M); and

5. The following large aromatic residues can substitute for each other: Phe (F), Tyr (Y), Trp (W).

The terms “corresponds with”, “corresponds to”, “aligns with” and the like can be used interchangeably herein. The relative position of a conserved amino acid motif (e.g., SEQ ID NO:67 or 66) in an arabinose isomerase herein can, for example, correspond with certain positions/residues (e.g., positions 237-269) that are associated with (define the location of) the conserved motif as it exists in a reference arabinose isomerase (e.g., SEQ ID NO:7). The position of the conserved motif of SEQ ID NO:67 or 66 in a particular arabinose isomerase can thus be determined with reference to positions 237-269 of SEQ ID NO:7, for example. In general, one can align the amino acid sequence of a query arabinose isomerase with SEQ ID NO:7 using an alignment algorithm and/or software described herein (e.g., BLASTP, ClustalW, ClustalV, Clustal Omega, EMBOSS) to determine if the conserved motif of SEQ ID NO:67 or 66, if present, is located at the noted position. In some embodiments, an alignment further indicates that SEQ ID NO:67 or 66 is at a position corresponding with positions 237-269 of SEQ ID NO:7, if the location of SEQ ID NO:67 or 66 (i) begins at any residue from positions 217-257, 227-247, 230-244, or 232-242, and (ii) ends at any residue from positions 249-289, 259-279, 262-276, or 264-274, of the amino acid sequence of the query arabinose isomerase.

The term “isolated” as used herein refers to a polynucleotide or polypeptide molecule that has been completely or partially purified from its native source. In some instances, the isolated polynucleotide or polypeptide molecule is part of a greater composition, buffer system or reagent mix. For example, an isolated polynucleotide or polypeptide molecule can be comprised within a cell or organism in a heterologous manner. Such a cell or organism containing heterologous components does not occur in nature, and/or exhibits properties not believed to naturally occur.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein. These terms can be used to characterize the “over-expression” or “up-regulation” of a polynucleotide encoding a protein, for example.

The present disclosure relates to engineered yeast strains that have arabinose isomerase activity. A challenge for engineering yeast to utilize arabinose, which is a sugar that can be obtained from cellulosic biomass, is to produce sufficient arabinose isomerase activity in the yeast cell. Arabinose isomerase catalyzes the conversion of arabinose to ribulose, which is the first step in an arabinose utilization pathway. Applicants have found that expression of specific arabinose isomerase polypeptides provides arabinose isomerase activity in yeast cells, while expression of other arabinose isomerase polypeptides does not provide activity. A yeast cell expressing arabinose isomerase activity provides a host cell for expression of a complete arabinose utilization pathway, thereby engineering a yeast cell that can produce a target chemical, such as ethanol, butanol, or 1,3-propanediol, using arabinose derived from lignocellulosic biomass as a carbon source, for example.

### Yeast Host Cells

Yeast cells of the present disclosure are those that comprise an arabinose isomerase (i.e., heterologous arabinose isomerase) that supports effective utilization of arabinose in an arabinose utilization pathway, and are capable of producing a target chemical. Preferred target chemicals are those of commercial value including, but not limited to, ethanol, butanol, or 1,3-propanediol.

Any yeast cells that either produce a target chemical, or can be engineered to produce a target chemical, may be used as host cells herein. Examples of such yeasts include, but are not limited to, yeasts of the genera Kluyveromyces, Candida, Pichia, Hansenula, Schizosaccharomyces (e.g., S. pombe), Kloeckera, Schwanniomyces, Yarrowia, and Saccharomyces (e.g., S. cerevisiae).

Yeast cells of the present disclosure comprising an effective arabinose isomerase may be engineered according to methods well known in the art. For example, yeast cells that have the native ability to produce ethanol from C6 sugars may be transformed with genes encoding C5 metabolic pathways including an arabinose isomerase disclosed herein. Such cells may be capable of either aerobic or anaerobic fermentation ethanol production.

In some embodiments, yeast cells may be engineered to express a pathway for synthesis of butanol or 1,3-propanediol. Engineering of pathways for butanol synthesis (including isobutanol, 1-butanol, and/or 2-butanol) have been disclosed, for example, in U.S. Pat. Nos. 8,206,970 and 7,851,188, and in U.S. Patent Appl. Publ. Nos. 2007/0292927, 2009/0155870 and 2008/0182308, all of which are incorporated herein by reference. Engineering of pathways for 1,3-propanediol have been disclosed in U.S. Pat. Nos. 6,514,733, 5,686,276, 7,005,291, 6,013,494 and 7,629,151, which are incorporated herein by reference.

For utilization of xylose as a carbon source, a yeast cell can be engineered for expression of a complete xylose utilization pathway. Engineering of yeast such as S. cerevisiae for production of ethanol from xylose is described in Matsushika et al. (Appl. Microbiol. Biotechnol. 84:37-53) and in Kuyper et al. (FEMS Yeast Res. 5:399-409), which are incorporated herein by reference. In certain embodiments, in addition to engineering a yeast cell to have xylose isomerase activity, the activities of other pathway enzymes are increased in the cell to provide the ability to grow on xylose. Typically the activity levels of five pentose pathway enzymes are increased: xylulokinase (XKS1), transaldolase (TAL1), transketolase 1 (TKL1), D-ribulose-5-phosphate 3-epimerase (RPE1), and ribose 5-phosphate ketol-isomerase (RKI1). Any method known to one skilled in the art for increasing expression of a gene may be used. For example, as described in the Examples (below), these activities may be increased by expressing a coding region for each protein using a highly active promoter. Chimeric genes for expression can be constructed and integrated into the yeast genome. Alternatively, heterologous coding regions for these enzymes may be expressed in the yeast cell to obtain increased enzyme activities. Other suitable methods for engineering yeast capable of metabolizing xylose have been disclosed in, for example, U.S. Pat. Nos. 7,622,284, 8,058,040, 8,129,171 and 7,943,366, International Patent Appl. Publ. Nos. WO2011153516A2, WO2011149353A1, WO2006115455A1 and WO2011079388A1, and U.S. Patent Appl. Publ. Nos. 2010/0112658, 2010/0028975, 2009/0061502, 2007/0155000 and 2006/0216804, all of which are incorporated herein by reference.

For utilization of arabinose as a carbon source, a yeast cell can be engineered to express a complete arabinose utilization pathway, as disclosed in U.S. Patent Appl. Publ. No. 2005/0142648 and U.S. Pat. No. 8,129,171, for example, which are incorporated herein by reference. To allow arabinose utilization, activities expressed in addition to activities of the xylose utilization pathway include: 1) L-arabinose isomerase (examples of which are presently disclosed) to convert L-arabinose to L-ribulose, 2) L-ribulokinase to convert L-ribulose to L-ribulose-5-phosphate, and 3) L-ribulose-5-phosphate-4-epimerase to convert L-ribulose-5-phosphate to D-xylulose. These enzyme activities can be expressed using coding regions of araA, araB, and araD genes, respectively. In certain aspects, the araB-encoded L-ribulokinase is from E. coli, and the araD-encoded L-ribulose-5-phosphate-4-epimerase is from E. coli. Any method known to one skilled in the art for expressing a foreign coding region may be used. For example, as described in the Examples (below), these activities can be expressed by introducing chimeric genes containing promoters active in yeast cells, heterologous codon-optimized coding regions for the enzymes, and termination sequences active in yeast cells.

### Arabinose Isomerase

Obtaining an effective amount of arabinose isomerase activity in yeast cells has been problematic. A group of arabinose isomerase enzymes were found herein that provide effective arabinose isomerase activity in a yeast cell for producing ethanol from arabinose in fermentation. The yeast cell, in addition to expressing the arabinose isomerase enzyme described herein, was genetically engineered as described above to express a xylose utilization pathway and a partial arabinose utilization pathway that lacks arabinose isomerase. The present arabinose isomerase was then expressed to complete the arabinose utilization pathway. One or more additional arabinose isomerases may also be expressed, if desired.

Twenty candidate arabinose isomerase enzymes (SEQ ID NOs:1-20) were chosen from the cow rumen metagenome dataset (Hess et al., Science 331:463-467) and the human microbiome dataset (The Human Microbiome Jumpstart Reference Strains Consortium et al., Science 328:994-999) as described in the Examples (below). Each of these arabinose enzymes was expressed in yeast cells from a codon-optimized coding sequence as described in Example 6 herein, and tested for the ability to support ethanol production by yeast cells grown in medium containing arabinose as the only sugar. Eight of the arabinose isomerase candidates (SEQ ID NOs:7, 8, 10, 15, 17, 18, 19 and 20) were effective in supporting production of ethanol, as compared to the arabinose isomerase from B. subtilis. Five of these arabinose isomerase candidates supported greater ethanol production as compared to the arabinose isomerase from B. subtilis: SEQ ID NOs:7, 10, 17, 18 and 19. Enzyme activities in protein extracts from the expressing strains showed these to be higher than for the B. subtilis arabinose isomerase, though the activities did not directly correlate with the ethanol production level of the corresponding strain in all cases. In some aspects, a yeast cell expressing an arabinose isomerase as presently disclosed can produce at least about 90%, 100%, 110%, 120%, 130%, 140%, or 150% of the amount of ethanol that is produced under suitable conditions by a yeast cell expressing a B. subtilis arabinose isomerase (e.g., SEQ ID NO:41).

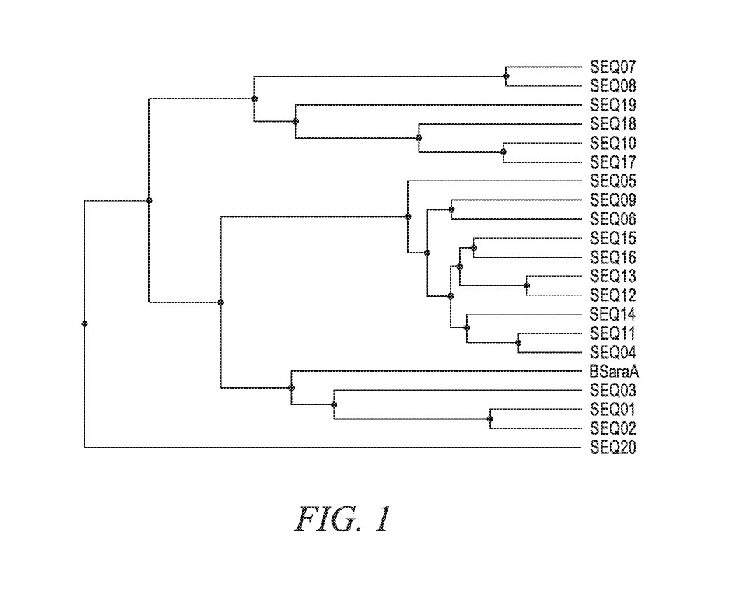

Six of the effective arabinose isomerase candidates (SEQ ID NOs:7, 8, 10, 17, 18 and 19) were found to be separated from the other candidates as members of one clade in a phylogenetic tree prepared for the twenty candidate arabinose isomerase enzyme amino acid sequences and the arabinose isomerase sequence from Bacillus subtilis (BSaraA; SEQ ID NO:41) (see FIG. 1). These six sequences were all from the cow rumen metagenome dataset. The sequences of SEQ ID NOs:7 and 8 have 94% amino acid sequence identity to each other. The sequences of SEQ ID NOs:10 and 17 have 92% amino acid sequence identity to each other. The sequences of SEQ ID NOs:10 and 17 have 80% amino acid sequence identity to SEQ ID NO: 18. Sequence identities among the other sequences are lower. Though the amino acid sequence identities vary among the six sequences of this clade, further sequence analysis identified an amino acid sequence motif (SEQ ID NO:67) that distinguishes the six sequences of this clade from the other fourteen candidate arabinose isomerase enzyme amino acid sequences, and that of the B. subtilis arabinose isomerase (SEQ ID NO:41). The distinguishing motif occurs starting with amino acid #237 and ending with amino acid #269, with reference to positions in the amino acid sequence of SEQ ID NO:7 (see FIGS. 2A and B). Based on the motif, this clade is called herein the “237-269 Motif” clade. The corresponding amino acid positions in different arabinose isomerase sequences can readily be determined by performing a sequence alignment. For example, these positions are #241 through #273 in both SEQ ID NOs:10 and 18.

The 237-269 Motif (FIG. 2A) is represented as formula I: I[RK]YQA[RK]EEIA[IM].K[IM][LM].[RA][EN]G[AC].AF.NTF[QE]DL . . . M (SEQ ID NO:67), where the standard single letter abbreviations for amino acids are used; where “[ ]” (brackets) indicate a position where the amino acid can be any one of the bracketed amino acids; and where each “.” (period mark) indicates a position that does not have distinguishing amino acids (i.e., the residue at such position may also be found at the corresponding position in a non-clade AI sequence) (e.g., can be any standard amino acid) in terms of physical properties.

Further information about the 237-269 Motif (FIG. 2B) is represented in formula II: I[RK]YQA[RK]EEIA[IM].K[IM][LM].[RA][EN]G[AC].AF.NTF[QE]DL . . . M (SEQ ID NO:67), where the standard single letter abbreviations for amino acids are used; where “[ ]” (brackets) indicate a position where the amino acid can be any one of the bracketed amino acids; where the letters in bold indicate positions that are conserved (for the specific amino acid or for the physiochemical properties of the amino acid in each particular bold position) among all twenty candidate sequences and are therefore not distinguishing for the motif; where each “.” (period mark) indicates a position that does not have distinguishing amino acids (i.e., the residue at such position may also be found at the corresponding position in a non-clade AI sequence) (e.g., can be any standard amino acid) in terms of physical properties; and where each underline indicates an amino acid that is only found at the underlined position in the present sequences (e.g., SEQ ID NOs:7, 8, 10, 17, 18, 19, or any other AI comprising SEQ ID NO:67) (i.e., they are not found in the non-clade sequences).

The amino acid sequence of the 237-269 Motif can be given as the following sequence, where the positions with non-distinguishing amino acids (“.” positions) are shown as bracketed multiple amino acids, any one of which may occur in these bracketed positions:

(SEQ ID NO: 66)
I[RK]YQA[RK]EEIA[IM][EK]K[IM][LM][VTD][RA][EN]G[AC]
[KNR]AF[SVT]NTF[EQ]DL[HIY]GM.

Thus, the 237-269 Motif as represented by SEQ ID NO:66 has an “Xaa” at each position having possible multiple amino acids, with the possible amino acids in these positions, as shown above in the bracket positions, designated in the present Sequence Listing.

There is variation in the amino acids of the motif among the six sequences of the 237-269 Motif clade (see FIG. 2B), however, the amino acid sequence of an arabinose isomerase can be readily identified by one skilled in the art as belonging to this clade based on overall matching with the motif. Thus, in certain embodiments, the present arabinose isomerase sequences for expression in yeast have the 237-269 Motif described above (e.g., SEQ ID NO:67 or SEQ ID NO:66) or a related sequence (e.g., a motif that is at least 90%, 91%, 92%, 93%, 94%, 95%, 96%, 97%, 98%, or 99% identical to SEQ ID NO:67 or SEQ ID NO:66). In some embodiments, an arabinose isomerase can comprise a motif that is at least 90%, 91%, 92%, 93%, 94%, 95%, 96%, 97%, 98%, or 99% identical to positions 237-269 of SEQ ID NO:7. The present arabinose isomerase sequences are any that belong to the 237-269 Motif clade (i.e., any that comprise SEQ ID NO:67, SEQ ID NO:66, or a related sequence thereof). It is noted for clarity that SEQ ID NO:66 is a version of SEQ ID NO:67.

In some embodiments, the present arabinose isomerase sequences are identified by specific amino acids matching to distinguishing positions in the motif sequence. The present arabinose isomerase is identified in this manner by having at least seventeen, eighteen, or nineteen of, or all of, the following amino acids in the motif: I at position 237; R or K at position 238; Y at position 239; R or K at position 242; E at position 243; I at position 245; A at position 246; I or M at position 247; K at position 249; I or M at position 250; R or A at position 253; E or N at position 254; G at position 255; A or C at position 256; F at position 259; N at position 261; T at position 262; Q or E at position 264; and M at position 269.

In some embodiments, the present arabinose isomerase sequences are identified by specific amino acids that are different from the amino acids at the corresponding positions in the sequences which are not in the 237-269 Motif clade. These specific amino acids are: E at position 243; I or M at position 250; A or C at position 256; and N at position 261.

In certain embodiments, an arabinose isomerase comprises, or consists of, an amino acid sequence that is 100% identical to, or at least about 80%, 85%, 90%, 91%, 92%, 93%, 94%, 95%, 96%, 97%, 98%, or 99% identical to, SEQ ID NO:7, 8, 10, 17, 18, or 19. Such an arabinose isomerase can optionally be further characterized to comprise SEQ ID NO:67 or SEQ ID NO:66 (or a variant of either motif that is at least 90% or 95% identical thereto), either of which being located at amino acid positions corresponding to positions 237-269 of SEQ ID NO:7. In some embodiments, an arabinose isomerase can (i) comprise, or consist of, an amino acid sequence that is at least about 80%, 85%, 90%, 91%, 92%, 93%, 94%, 95%, 96%, 97%, 98%, or 99% identical to, SEQ ID NO:7, 8, 10, 17, 18, or 19, and (ii) comprise the respective motif shown in FIG. 2C.

In some embodiments, an arabinose isomerase can comprise a motif that is at least about 70%, 75%, 80%, 85%, 90%, 91%, 92%, 93%, 94%, 95%, 96%, 97%, 98%, or 99% identical to positions 237-269 of SEQ ID NO:7. Such an arabinose isomerase can optionally further be characterized as comprising, or consisting of, an amino acid sequence that is at least about 80%, 85%, 90%, 91%, 92%, 93%, 94%, 95%, 96%, 97%, 98%, or 99% identical to SEQ ID NO:7, 8, 10, 17, 18, or 19.

In some embodiments, an arabinose isomerase comprises, or consists of, the amino acid sequence of (i) SEQ ID NO:10, 17, 18, or 19 (or any variant thereof as disclosed herein) or (ii) SEQ ID NO:10, 17, or 18 (or any variant thereof as disclosed herein). An arabinose isomerase, in some aspects of the present disclosure, does not comprise SEQ ID NO:15, 68, 69, 70, or 71.

The present amino acid sequences that provide arabinose isomerase activity in yeast cells are not native to yeast cells, thus their encoding polynucleotide sequences are heterologous to yeast cells. For expression, polynucleotide molecules encoding the present polypeptides may be designed using codon optimization for the desired yeast cell. For example, to express SEQ ID NO:7, 8,10, 17, 18, and/or 19, a codon-optimized coding region of SEQ ID NO:27, 28, 30, 37, 38, and/or 39 can be used, respectively. A polynucleotide can also be characterized as being heterologous in some aspects by virtue of comprising heterologously combined elements (e.g., a promoter that is heterologous to the sequence encoding the polypeptide).

Methods for gene expression in yeasts are known in the art (see for example Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.). Expression of genes in yeast typically requires a promoter, operably linked to the coding region of interest, and a transcriptional terminator. A number of yeast promoters can be used in constructing expression cassettes for genes encoding the desired proteins, including, but not limited to, constitutive promoters (e.g., FBA1, GPD1, PDC1, ADH1, GPM, TPI1, TDH3, PGK1, ILV5p) and inducible promoters (e.g., GAL1, GAL10, CUP1). Suitable transcription terminators include, but are not limited to, FBAt, GPDt, GPMt, ERG10t, GAL1t, CYC1t, ADH1t, TAL1t, TKL1t, ILV5t, and ADHt.

A polynucleotide sequence herein encoding an arabinose isomerase can be a vector (e.g., plasmid, cosmid) containing a selectable marker and sequences allowing autonomous replication or chromosomal integration in the desired host, for example. Typically used plasmids in yeast are shuttle vectors pRS423, pRS424, pRS425, and pRS426 (American Type Culture Collection, Rockville, Md.), which contain an E. coli replication origin (e.g., pMB1), a yeast 2μ origin of replication, and a marker for nutritional selection. The selection markers for these four vectors are His3 (vector pRS423), Trp1 (vector pRS424), Leu2 (vector pRS425) and Ura3 (vector pRS426). Additional vectors that may be used include pHR81 (ATCC #87541) and pRS313 (ATCC #77142). Construction of expression vectors with chimeric genes encoding the desired proteins may be performed by either standard molecular cloning techniques in E. coli or by the gap repair recombination method in yeast, for example.

The present disclosure also provides a method for producing a yeast cell that has arabinose isomerase activity following the teachings above. In this method, a heterologous polynucleotide encoding an arabinose isomerase as presently disclosed is introduced into a yeast cell lacking arabinose isomerase. Any yeast cell as disclosed herein can be produced using this method, if desired. Some aspects are drawn to increasing the arabinose isomerase activity of a yeast cell that already comprises a heterologous arabinose isomerase, by introducing a polynucleotide encoding an arabinose isomerase as presently disclosed to the yeast cell.

In various embodiments, a heterologous polynucleotide encoding a polypeptide having arabinose isomerase activity can be introduced into the yeast cell before, after, or at the same time that other genes for expressing enzymes of an arabinose utilization pathway are introduced.

In certain embodiments, a heterologous polynucleotide herein can be introduced into a yeast cell that has already been modified to comprise a complete xylose utilization pathway. Introduction of arabinose isomerase activity and additional modifications for a xylose utilization pathway and a complete arabinose utilization pathway may be performed in any order, and/or with two or more introductions/modifications performed concurrently. Such yeast cells have the ability to grow in medium containing arabinose as the sole carbon source. More typically, these cells are grown in medium containing arabinose, as well as other sugars such as glucose and/or xylose. This latter growth scheme allows effective use of the sugars found in a hydrolysate medium prepared from cellulosic biomass by pretreatment and saccharification. Some embodiments therefore are drawn to a fermentation comprising at least a yeast cell as disclosed herein, arabinose, and optionally xylose and/or glucose. Any or all of the sugar components of such a fermentation can be provided from a lignocellulosic biomass hydrolysate, for example.

In certain embodiments, a heterologous polynucleotide herein can be introduced into a yeast cell that has a metabolic pathway that produces a target chemical. The pathway may be endogenous, or it may be engineered in the cell. Introduction of arabinose isomerase activity and a metabolic pathway producing a target chemical may be performed in any order, and/or with two or more genetic modifications performed concurrently. Examples of target chemicals include ethanol, butanol, and 1,3-propanediol. Yeast cells containing metabolic pathways for production of target chemicals are described above, for example.

### Production of a Target Chemical Using Arabinose

The present yeast cells expressing an arabinose isomerase as part of an arabinose utilization pathway, and producing ethanol and/or another target chemical, can be grown in medium containing arabinose. Typically, such yeast cells also are able to utilize xylose and the medium contains additional sugars such as glucose and xylose. In certain embodiments, lignocellulosic biomass hydrolysate is used in fermentation medium, for example as disclosed in U.S. Pat. No. 7,932,063, which is incorporated herein by reference.

A variety of culture methodologies may be applied. For example, large-scale fermentation/production may use batch, fed-batch, or continuous culture methodologies. Other fermentation conditions such as pH, oxygenation, and temperature can be applied, accordingly.

In any embodiment disclosed herein, an arabinose isomerase can, instead of being one as disclosed herein to comprise a 237-269 Motif (e.g., SEQ ID NO:66 or 67), rather be one comprising, or consisting of, an amino acid sequence that is 100% identical to, or at least about 80%, 85%, 90%, 91%, 92%, 93%, 94%, 95%, 96%, 97%, 98%, or 99% identical to, SEQ ID NO:15 or 20. Such enzymes do not belong to the 237-269 Motif clade.

Non-limiting examples of compositions and methods disclosed herein include:

1. A recombinant yeast cell comprising an arabinose utilization pathway that comprises a polypeptide having arabinose isomerase activity, wherein the yeast cell comprises a heterologous polynucleotide encoding the polypeptide, wherein the polypeptide comprises a motif that is at least 90% identical to SEQ ID NO:67, and wherein the position of the motif in the polypeptide corresponds with positions 237-269 of SEQ ID NO:7.
2. The recombinant yeast cell of embodiment 1, wherein the motif comprises at least seventeen amino acids selected from the group consisting of: (a) I at position 237; (b) R or K at position 238; (c) Y at position 239; (d) R or K at position 242; (e) E at position 243; (f) I at position 245; (g) A at position 246; (h) I or M at position 247; (i) K at position 249; (j) I or M at position 250; (k) R or A at position 253; (l) E or N at position 254; (m) G at position 255; (n) A or C at position 256; (o) F at position 259; (p) N at position 261; (q) T at position 262; (r) Q or E at position 264; and (s) M at position 269; wherein each position of (a)-(s) corresponds with the respective position in positions 237-269 of SEQ ID NO:7.
3. The recombinant yeast cell of embodiment 1 or 2, wherein the polypeptide comprises a motif that is SEQ ID NO:67.
4. The recombinant yeast cell of embodiment 1, 2, or 3, wherein the polypeptide comprises a motif that is at least 90% identical to SEQ ID NO:66.
5. The recombinant yeast cell of embodiment 4, wherein the polypeptide comprises a motif that is SEQ ID NO:66.
6. The recombinant yeast cell of embodiment 1, 2, 3, 4, or 5, wherein the polypeptide comprises an amino acid sequence that is at least 85% identical to SEQ ID NO:7, 8, 10, 17, 18, or 19.
7. The recombinant yeast cell of embodiment 1, 2, 3, 4, 5, or 6, further comprising a metabolic pathway that produces a target compound, optionally wherein the target compound is ethanol, butanol, or 1,3-propanediol.
8. A method for producing a yeast cell having arabinose isomerase activity, the method comprising: (a) providing a yeast cell lacking arabinose isomerase; and (b) introducing a heterologous polynucleotide into the yeast cell, wherein the heterologous polynucleotide encodes a polypeptide having arabinose isomerase activity, and wherein the polypeptide comprises a motif that is at least 90% identical to SEQ ID NO:67.
9. The method of embodiment 8, wherein: (i) the yeast cell of step (a) comprises one or more polynucleotides encoding enzymes, except an arabinose isomerase, of an arabinose utilization pathway, or (ii) step (b) further comprises introducing, into the yeast cell, one or more polynucleotides encoding enzymes of an arabinose utilization pathway, wherein this further introduction is at the same time of, or after, the introducing the heterologous polynucleotide encoding the polypeptide having arabinose isomerase activity.
10. A method of producing a target compound from arabinose comprising: (a) providing the recombinant yeast cell of any one of embodiments 1-7; (b) growing the yeast cell of (a) in medium comprising arabinose, wherein the target compound is produced; and c) optionally isolating the target compound of (b).
11. The method of embodiment 10, wherein the target compound is ethanol, butanol, or 1,3-propanediol.
12. A recombinant yeast cell comprising an arabinose utilization pathway that comprises a polypeptide having arabinose isomerase activity, wherein the yeast cell comprises a heterologous polynucleotide encoding the polypeptide, wherein the polypeptide comprises an amino acid sequence that is at least 85% identical to SEQ ID NO:15 or 20.
13. The recombinant yeast cell of embodiment 12, further comprising a metabolic pathway that produces a target compound, optionally wherein the target compound is ethanol, butanol, or 1,3-propanediol.
14. A method of producing a target compound from arabinose comprising: (a) providing the recombinant yeast cell of embodiment 12 or 13; (b) growing the yeast cell of (a) in medium comprising arabinose, wherein the target compound is produced; and c) optionally isolating the target compound of (b).
15. A method for producing a yeast cell having arabinose isomerase activity, the method comprising: (a) providing a yeast cell lacking arabinose isomerase; and (b) introducing a heterologous polynucleotide into the yeast cell, wherein the heterologous polynucleotide encodes a polypeptide (i) having arabinose isomerase activity and (ii) comprising an amino acid sequence that is at least 85% identical to SEQ ID NO:15 or 20.

### EXAMPLES

The present disclosure is further exemplified in the following Examples. It should be understood that these Examples, while indicating certain preferred aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### Example 1

### Plasmid Constructs for Xylose Utilization Pathway

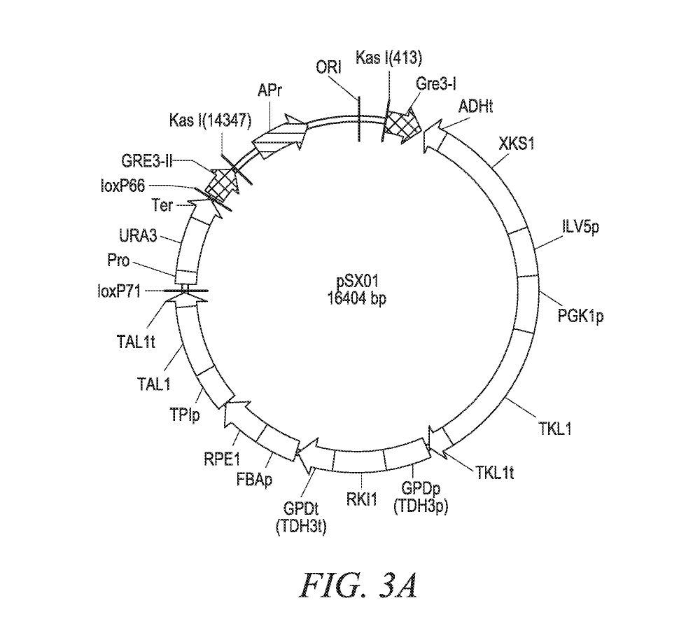

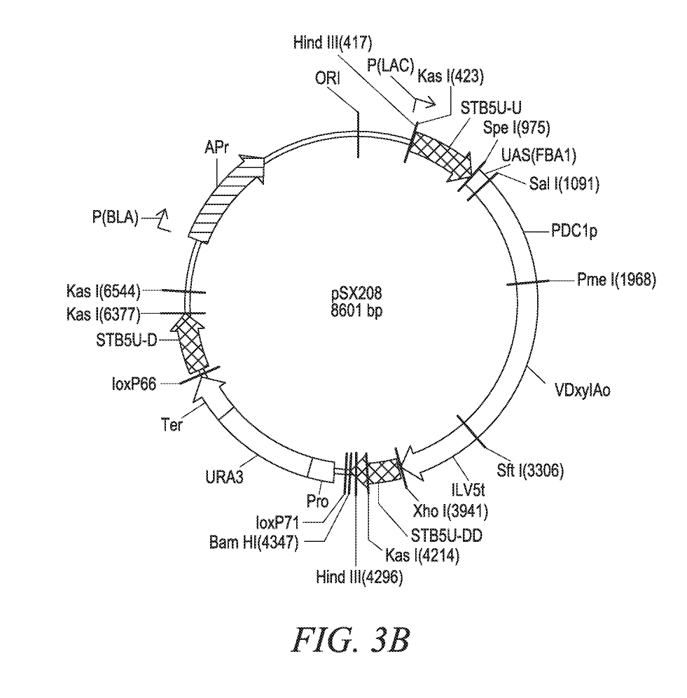

To make xylose-utilizing yeast strains, a plasmid for increasing expression of the pentose pathway in Saccharomyces cerevisiae that was described as P5 Integration Vector in GRE3 in U.S. Pat. No. 8,669,076 (Example 1 therein), which is incorporated herein by reference, was used. This plasmid was renamed herein as pSX01 (SEQ ID NO:43; FIG. 3A). It contains a 12719-bp P5 transgene fragment having five chimeric genes (XKS1, TKL1, RKI1, RPE1, and TAL1) and a URA3 marker, flanked by a pair of homologous recombination fragments (HRF), GRE3-I (572-bp) and GRE3-II (541-bp). GRE3-I and GRE3-II direct integration of the transgene fragment into the GRE3 locus on chromosome 8 of the S. cerevisiae genome, between positions 323809 and 324118. Integration truncates the GRE3 coding sequence between nucleotides 401 and 710, which removes a 308-bp sequence from it. On the transgene fragment, TKL1, RKI1, RPE1, and TAL1 encode four pentose phosphate pathway enzymes: transketolase (EC 2.2.1.1), ribose-5-phosphate ketokisornerase (EC 5.3.1.6), ribulose-phosphate 3-epimerase (EC 5.1.3.1), and transaldolase (EC 2.2.1.2); XKS1 encodes a xylose assimilation pathway enzyme xylulokinase (EC 2.7.1.17); URA3 functions as a selection marker for transformation of the URA3-deletion strains. A pair of Lox elements was located at 5′ and 3′ ends of the URA3 marker so that it could be removed after integration of the transgene, when a Cre recombinase is introduced to transformants.

Xylose isomerase (EC 5.3.1.5) is a key enzyme for the xylose assimilation pathway in many bacteria and a few fungi. It is a slow enzyme with poor kinetic properties. It was previously disclosed in U.S. Pat. Nos. 8,114,974 and 8,093,037, which are incorporated herein by reference, that a synthetic xylA gene (herein named VDxylA) was expressed and functioned well in S. cerevisiae. VDxylA has the amino acid sequence shown in SEQ ID NO:44. A 1323-bp VDxylA coding sequence was synthesized using codon optimization for expression in S. cerevisiae (SEQ ID NO:45). It was then linked with a 868-bp PDC1 promoter and 110-bp UAS(FBA1) enhancer at its 5′ end and a 623-bp ILV5 terminator at its 3′ end, forming a 2966-bp chimeric expression cassette designated as UAS(FBA1)::PDC1p::VDxylA::ILV5t (SEQ ID NO:46).

The VDxylA cassette was constructed into three different integration plasmids, which targeted for three integration loci specifically. These plasmids had a 2653-bp common backbone sequence, which contained an E. coli replication origin (ORI) and ampicillin-resistance marker (AP^{r}) for plasmid propagation in E. coli, as well as KasI sites at both ends. The transgene sequences had a structure of HRF-UNDxylA Cassette/HRF-DD/URA3 Cassette/HRF-D. They were connected with the backbone through KasI sites. In this transgene structure, HRF-U and HRF-D were two homologous recombination fragments able to integrate the transgene into the S. cerevisiae chromosome between the sequences corresponding to these two HRFs. The URA3 cassette provided a selective marker for integration. HRF-DD was a third homologous recombination fragment corresponding to a chromosomal sequence further downstream of HRF-D. After integration, this fragment was able to interact with the chromosomal copy of HRF-DD to loop-out the URA3 cassette and the HRF-D, thus leaving the VDxylA cassette between HRF-U and HRF-DD on the chromosome without a selective marker.

Plasmid pSX208 (SEQ ID NO:47; FIG. 3B) has a VDxylA cassette in its transgene fragment. Its three HRFs are 546-bp STB5U-U, 487-bp STB5U-D, and 386-bp STB5U-DD, corresponding to the S. cerevisiae chromosome-VIII from coordinates 457706 to 458251, from coordinates 458334 to 458820, and from coordinates 458836 to 459221, respectively. Therefore, during integration, the transgene fragment inserts into chromosome-VIII between coordinates 458251 and 458334. After recycling the URA3 marker, the VDxylA cassette was located between coordinates 458251 and 458836, that is, in an intergenic region upstream of the STB5 locus (YHR178W).

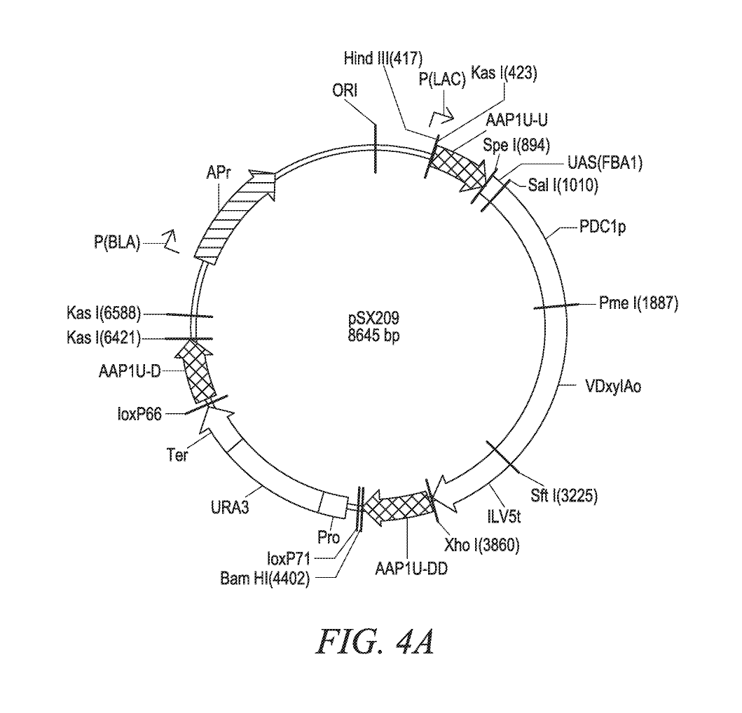

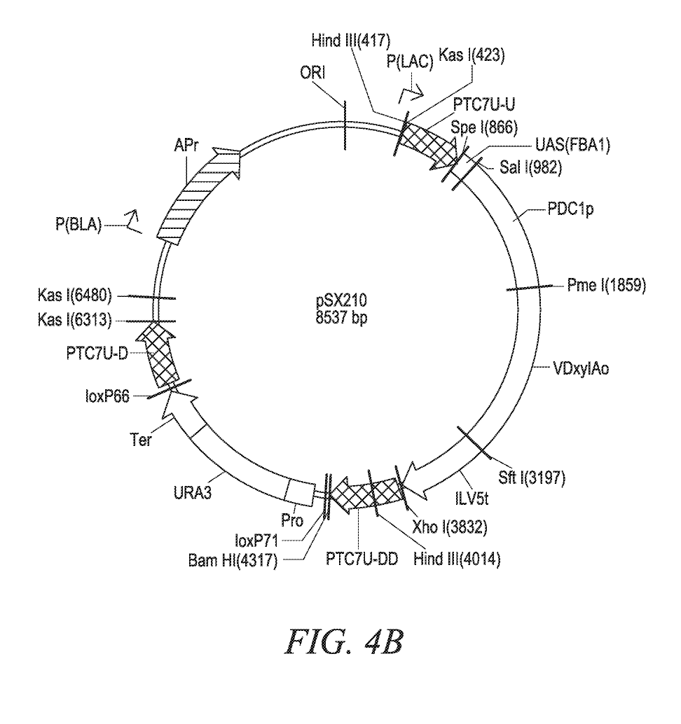

Plasmid pSX209 (SEQ ID NO:48; FIG. 4A) has a VDxylA cassette in its transgene fragment. Its three HRFs are 465-bp AAP1U-U, 476-bp AAP1U-D, and 522-bp AAP1U-DD, corresponding to S. cerevisiae chromosome-VIII from coordinates 203581 to 203117, from coordinates 202845 to 202370, and from coordinates 202362 to 201841, respectively. Therefore, during integration, the transgene fragment inserts into chromosome-VIII between coordinates 203117 and 202845. After recycling the URA3 marker, the VDxylA cassette from pSX209 was located between coordinates 203117 and 202362, that is, in an intergenic region upstream of the AAP1 locus (YHR047C).

Plasmid pSX210 (SEQ ID NO:49; FIG. 4B) has a VDxylA cassette in its transgene fragment. Its three HRFs are 437-bp PTC7U-U, 453-bp PTC7U-D, and 465-bp PTC7U-DD, corresponding to S. cerevisiae chromosome-VIII from coordinates 249702 to 250138, from coordinates 250199 to 250651, and from coordinates 250670 to 251134, respectively. Therefore, during integration, the transgene fragment inserts into chromosome-VIII between coordinates 250138 and 250199. After recycling the URA3 marker, the VDxylA cassette from pSX210 was located between coordinates 250138 and 250670, that is, in an intergenic region upstream of the PTC7 locus (YHR076W). Table 2 is a summary of plasmids pSX208, pSX209, and pSX210.

TABLE 2
Summary of pSX208, pSX209 and pSX210 Constructs.

| | pSX208 | pSX209 | pSX210 |
|---|---|---|---|
| Size | 8601 bp | 8645 bp | 8537 bp |
| Backbone | 6376-427 | 6420-427 | 6312-427 |
| STB5U-U | 428-973 | 428-892 | 428-864 |
| VDxylA Cassette | 980-3945 | 899-3864 | 871-3836 |
| STB5U-DD | 3960-4345 | 3879-4400 | 3851-4315 |
| URA3 Cassette | 4472-5826 | 4527-5881 | 4442-5796 |
| STB5U-D | 5889-6375 | 5944-6419 | 5859-6311 |

### Example 2

### Development of Xylose-Utilization Strains

To make xylose-utilizing recombinant strains, the S. cerevisiae strain PXI3 was used as the recipient of transgenes. PXI3, also called BP1548, is a CEN.PK-based haploid laboratory strain derived from prototrophic diploid strain CBS 8272 (Centraalbureau voor Schimmelcultures [CBS] Fungal Biodiversity Centre, Netherlands), with a genotype of MATα ura3Δ his3Δ. The strain was described in detail in Patent Appl. Publ. No. 2014/0178954 (Example 5 therein), which is incorporated herein by reference. Both native URA3 coding sequences were deleted by an approach similar to that described in Patent Appl. Publ. No. 2014/0178954.

The 12719-bp P5 transgene fragment (see Example 1), which included the homologous recombination regions GRE3-I and GRE3-II, was isolated from plasmid pSX01 by KasI digestion and transformed into the PXI3 strain using the FROZEN-EZ Yeast Transformation II Kit from Zymo Research (Irvine, Calif.). Transformants were selected on plates with CM/Gluc/−Ura (Teknova), which is a synthetic dropout (SD) medium lacking uracil. The URA3 marker was removed by introducing a CRE recombinase vector, pJT254 (SEQ ID NO:50), into the transformants. This vector was derived from pRS413 and the cre coding region (nt 2562 to 3593) was under the control of the GAL1 promoter (nt 2119 to 2561). Strains that could no longer grow on SD (−uracil) medium were selected. Further passages on YPD medium (Teknova) were used to cure the pJT254 plasmid. The resulting CEN-PK-based strain was named PX112. It has a P5 transgene fragment integrated into GRE3 locus as described in Example 1, with a genotype of MATα ura3Δ his3Δ gre3Δ:P5.

To integrate the xylA gene into the PX112 strain, the transgene fragments with the flanked HRFs were amplified by PCR from pSX208 or isolated from pSX209 and pSX210 by KasI digestion. The fragments were sequentially transformed into the strain to achieve multiple insertions. For one round of integration, the xylA transgene fragment was transformed into the recipient strain using the FROZEN-EZ Yeast Transformation II Kit from Zymo Research. Transformants were selected on CM/Gluc/−Ura Plates. Accurate integration was confirmed by PCR. Integration was followed by a recycling procedure to remove the URA3 marker. For this purpose, the transformant was grown in YPD broth (Teknova) overnight and then on a CM-FOA plate (6.7 g/L Sigma yeast nitrogen base without amino acids, 0.77 g/L Clontech dropout mix without uracil, 20 g/L glucose, 40 g/L uracil, 1 g/L 5-fluoro-orotic acid, 20 g/L agar) for two days. The survivors were streaked on both a YPD plate and a CM/Gluc/−Ura plate. A marker-free transformant was identified when it grew on the YPD plate but not on the CM/Gluc/−Ura plate. Marker removal was confirmed by PCR. When the transgene fragments of pSX208, pSX209, and pSX210 were integrated sequentially into PX112, a CEN-PK based haploid strain was obtained, which has a genotype of MATα ura3Δ::loxP his3Δ gre3Δ:P5 AAP1UΔ::UAS(FBA1)-PDC1p-VDxylA-ILV5t PTC7UΔ::UAS(FBA1)-PDC1p-VDxylA-ILV5t STB5UΔ::UAS(FBA1)-PDC1p-VDxylA-1 LV5t. The resulting strain was named PXI68.

PXI68 was subjected to adaptation to speed up xylose fermentation. Adaptation was carried out in 4 mL YPX4 broth (10 g/L yeast extract, 20 g/L peptone, 4 g/L xylose) with a starting cell density at an OD₆₀₀ value of 0.5. The strain was grown under micro-aerobic conditions at 32° C. and 200 rpm shaking until approximately 5 doublings. Then it was passed to fresh YPX4 and grown under the same conditions. Adaptation was completed after 10 passages (approximately 50 doublings). Individual adapted strains were isolated and examined for improved xylose utilization using a standard mini-fermentation assay. This assay is described in Example 4 below, using YPX4 (YP medium containing 40 g/L xylose) as fermentation broth. The adaptation resulted in the improved strain, PXI82.

### Example 3

### Plasmid Constructs for Arabinose Utilization Pathway

To assemble a bacterium-type arabinose assimilation pathway in a xylose-utilizing strain so that the resultant strain is able to utilize glucose, xylose, and arabinose, three enzymes are required. L-arabinose isomerase (EC 5.3.1.4) is a key enzyme for the arabinose assimilation pathway in many bacteria, encoded by the araA gene. Similar to xylose isomerase, it is a slow enzyme with poor kinetic properties. The B. subtilis araA (BSaraA) gene-encoded arabinose isomerase has the amino acid sequence shown in SEQ ID NO:41. To express this araA, a 1491-bp BSaraA coding region was synthesized using codon optimization for expression in S. cerevisiae (SEQ ID NO:42). It was then linked with the 678-bp ADP promoter (ADHp) at its 5′ end and with the 252-bp CYC1 terminator at its 3′ end, forming a 2429-bp chimeric expression cassette called ADHp::BSaraA::CYC1t (SEQ ID NO:51) or BSaraA cassette. L-ribulokinase (EC 2.7.1.16) is the second enzyme in the bacterial arabinose assimilation pathway, encoded by the araB gene. The E. coli araB (ECaraB) gene-encoded ribulokinase has the amino acid sequence shown in SEQ ID NO:52. To express this araB, a 1701-bp ECaraB coding region was synthesized using codon optimization for expression in S. cerevisiae (SEQ ID NO:53). It was then linked with the 700-bp ILV5 promoter (ILV5p) at its 5′ end and with the 500-bp PHO13 terminator (PHO13-3′UTR) at its 3′ end, forming a 2907-bp chimeric expression cassette called ILV5p::ECaraB::PHO13-3′UTR (SEQ ID NO:54) or ECaraB cassette. L-ribulose-5-phosphate 4-epimerase (EC 5.1.3.4) is the third enzyme in the bacterial arabinose assimilation pathway, encoded by the araD gene. The E. coli araD (ECaraD) gene-encoded L-ribulose-5-phosphate 4-epimerase has the amino acid sequence shown in SEQ ID NO:55. To express this araD, a 696-bp ECaraD coding region was synthesized using codon optimization for expression in S. cerevisiae (SEQ ID NO:56). It was then linked with the 679-bp GPD promoter (GPDp) at its 5′ end and with the 316-bp ADH1 terminator (ADP1t) at its 3′ end, forming a 1691-bp chimeric expression cassette called GPDp::ECaraD::ADH1t (SEQ ID NO:57) or ECaraD cassette.

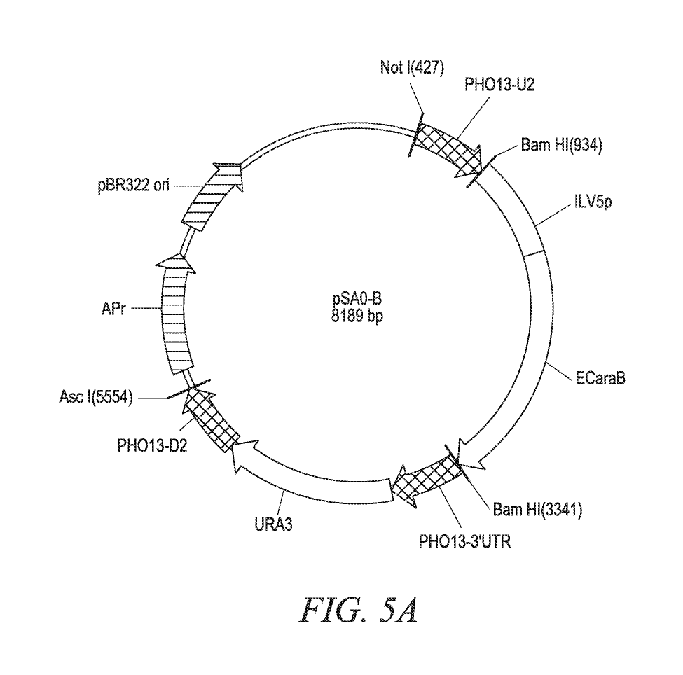

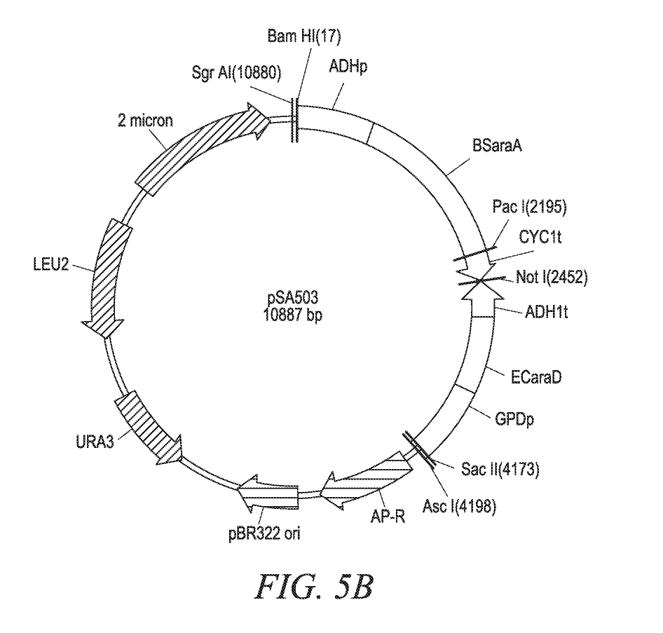

The ECaraB cassette was constructed into an integration plasmid, which targeted integration in the PHO13 locus (YDL236W, encoding for an alkaline phosphatase specific for p-nitrophenyl phosphate) on chromosome-IV, resulting in pSA0-B (SEQ ID NO:58; FIG. 5A). Similar to the integration plasmid described earlier, the backbone of this plasmid contained an E. coli replication origin (pBR322 ori) and ampicillin-resistance marker (AP^{r}) for plasmid propagation in E. coli, as well as AscI and NotI sites at the ends. In the integration plasmid, transgene sequences had a structure of PHO13-U2/ECaraB Cassette/PHO13-3′UTR/URA3 Cassette/PHO13-D2. It was connected with the backbone through NotI and AscI sites. In this transgene structure, 500-bp PHO13-U2 and 500-bp PHO13-D2 were two homologous recombination fragments, corresponding to the chromosome-IV sequence from coordinates 31794 to 32294 and from coordinates 32735 to 33234, respectively. These sequences direct integration of the transgenes into chromosome-IV between the sequences corresponding to these two HRFs, which interrupted the PHO13 locus and deleted the first 439 nucleotides in the coding region. Within the transgene structure, the URA3 cassette provided a selective marker for integration. The PHO13-3′UTR not only served as a terminator for ECaraB but also was the third homologous recombination fragment corresponding to a chromosomal sequence further downstream of PHO13-D2, from coordinates 33235 to 33734. After integration, this fragment was able to interact with the chromosomal copy of PHO13-3′UTR to loop-out the URA3 Cassette and PHO13-D2, thus leaving the ECaraB cassette between PHO13-U2 and PHO13-3′UTR on chromosome-IV without a selective marker. The marker recycling of URA3 further removed the rest of the PHO13 coding sequence. Therefore, the entire PHO13 coding sequence was completely deleted.

The BSaraA cassette and ECaraD cassette were constructed into a high copy number shuttle vector, resulting in pSA503 (SEQ ID NO:59, FIG. 5B) that could support a high level transient expression of these transgenes. The backbone of this plasmid contained an E. coli replication origin (pBR322 ori) and ampicillin-resistance marker (AP^{r}) for plasmid propagation in E. coli. It also contained an S. cerevisiae 2 micron replication sequence, and LEU2 and URA3 selection markers for plasmid propagation in S. cerevisiae. The BSaraA cassette was located downstream of the 2 micron element, with a unique 5′ BamHI site. The ECaraD cassette was located downstream of the BSaraA cassette but in opposite orientation, with a unique 5′ SacII site. The two cassettes were separated by a unique NotI site.

### Example 4

### Development of Arabinose-Utilization Strains

To make arabinose-utilizing recombinant strains, PX182 (prepared in Example 2) was used as the recipient of transgenes. The transgene fragment of PHO13-U2/ECaraB Cassette/PHO13-3′UTR/URA3 Cassette/PHO13-D2 was isolated from plasmid pSA0-B by NotI and AscI digestion and transformed into the PX182 strain using the FROZEN-EZ Yeast Transformation II Kit from Zymo Research (Irvine, Calif.). Transformants were selected on CM/Gluc/−Ura plates. Accurate integration was confirmed by PCR. Integration was followed by a recycling procedure to remove the URA3 marker. For this purpose, transformants were grown in YPD broth overnight and then on CM-FOA plates (6.7 g/L Sigma yeast nitrogen base without amino acids, 0.77 g/L Clontech dropout mix without uracil, 20 g/L glucose, 40 g/L uracil, 1 g/L 5-fluoro-orotic acid, 20 g/L agar) for two days. The survivors were streaked on both YPD plates and CM/Gluc/−Ura plates. A marker-free transformant was identified when it grew on a YPD plate but not on a CM/Gluc/−Ura plate, and was named PX182-araB. Marker removal was confirmed by PCR. To introduce the BSaraA cassette and the ECaraD cassette into the strain, shuttle vector pSA503 was transformed into the PX182-araB strain using the FROZEN-EZ Yeast Transformation II Kit from Zymo Research. Transformants were selected on CM/Gluc/−Ura Plates and named PX182-araBAD5030.

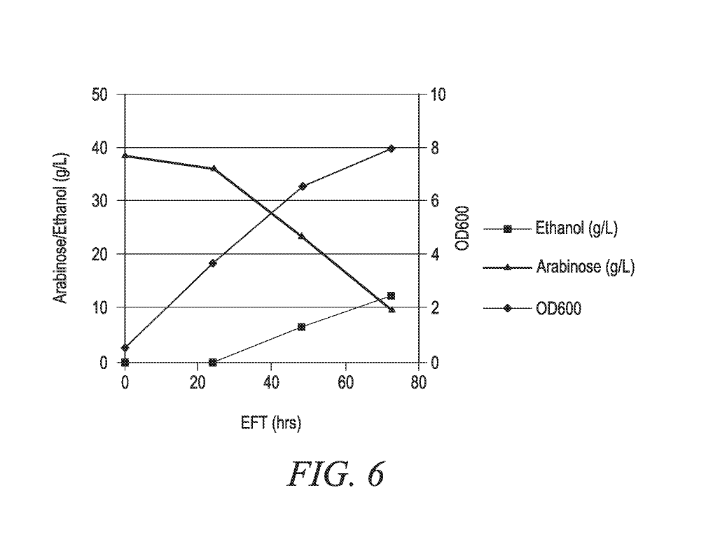

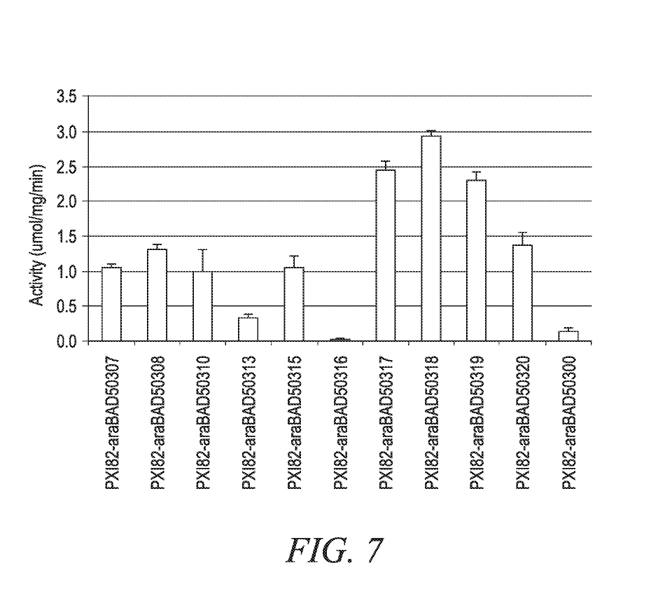

The PX182-araBAD5030 strain was tested for arabinose assimilation and fermentation capacity in a mini-shaking bottle fermentation under micro-aerobic conditions. Fermentation broth was YPA4 (YP medium containing 40 g/L arabinose). To assemble the fermentation, the strain was grown in 3 mL YPD culture at 32° C. with shaking at 200 rpm overnight. Fresh cells were added into 5-mL NALGENE PETG diagnostics vials containing 4 mL fermentation broth, with a starting OD₆₀₀ at 0.5. The PETG vials were sealed by screw caps mounted with a WHEATON 20-mm SEPTA PTEF red rubber pad within it. Micro-aerobic conditions were achieved by inserting BD 26G needles through the caps. Fermentation was conducted at 32° C. with shaking at 200 rpm. At specified time intervals, 0.5 mL of culture was drawn through the needle using a syringe. One fifth of the collected culture (0.1 mL) was used for measurement of OD₆₀₀. The rest of the culture was spun in a micro-centrifuge at 14000 rpm for 2 min. The supernatant was carefully collected by pipette, placed into a 0.22-μm COSTAR SPIN-X Centrifuge Tube Filter (Corning Inc., Corning, N.Y.), and then passed through the filter by micro-centrifuging at 6000 rpm for 2 min. The flow-through was loaded into an AGILENT 250-4 vial insert within a 2-mL crimp vial and sealed. Xylose, ethanol (EtOH) and other metabolites were analyzed by running flow-through samples through a BIORAD AMINEX HPX-A7H ion exclusion column with 0.01 N H₂SO₄ at a speed of 0.6 mL/min at 55° C. on an AGILENT 1100 HPLC system. Assay results (FIG. 6) showed that during 72-hr of fermentation, arabinose concentration in the broth was reduced from 38.5 g/L to 10.1 g/L (73.7% arabinose had been consumed). The arabinose consumption resulted in cell growth from OD₆₀₀ value of 0.5 to 7.9, and supported production of 12.4 g/L ethanol. It confirmed that an arabinose assimilation pathway was assembled successfully in the PX182-araBAD5030 strain. This pathway, as combined with the pentose phosphate pathway engineered into the strain previously, was able to ferment arabinose to ethanol.

### Example 5

### Identification of AraA Candidates from Cow Rumen and Human Microbiome Databases

Arabinose isomerase is slow enzyme and functions in the first step of the bacterial-type arabinose assimilation pathway. To identify new bacterial arabinose isomerase candidates for expression testing in yeast, we used amino acid sequences of the arabinose isomerases from seven bacteria species: Bacillus subtilis (BS; SEQ ID NO:41), Escherichia coli (EC; SEQ ID NO:60), Bacillus licheniformis (BL; SEQ ID NO:61), Clostridium acetobutylicum (CA; SEQ ID NO:62), Leuconostoc mesenteroides (LM; SEQ ID NO:63), Lactobacillus plantarum (LP; SEQ ID NO:64), and Pediococcus pentosaceus (PP; SEQ ID NO:65) as queries in BLAST searches against translated open reading frames of the databases generated from the cow rumen metagenome dataset (Hess et al., Science 331:463-467) and the human microbiome dataset (The Human Microbiome Jumpstart Reference Strains Consortium et al., Science 328: 994-999). The putative open reading frames in the BLAST search results were first filtered by removing those entries with greater than 70% identity to any of the seven query amino acid sequences, or those entries containing one or more ambiguous nucleotide “N” in the nucleotide sequences. As all seven query arabinose isomerases have an L-arabinose isomerase protein domain (Arabinose_Isome, PFAM identifier PF02610) followed by an L-arabinose isomerase C-terminal protein domain (Arabinose_Iso_C, PFAM identifier PF11762), we further removed any open reading frames in the BLAST search results which did not contain both aforementioned protein domains in the same order. From the remaining search results, nine putative arabinose isomerases from among the sequences with the closest identities to B. subtilis arabinose isomerase were chosen from the cow rumen metagenome dataset (CR), and eleven were chosen from the human microbiome dataset (HM). These twenty arabinose isomerase (AI) candidates are listed and their sequence identity comparisons with the seven query arabinose isomerases are given in Table 3.

TABLE 3
Al Candidates and Their Sequence Comparison with Known Als.

| | Data | SEQ | Percent Identity to Seven Known Als | | | | | | |
|---|---|---|---|---|---|---|---|---|---|
| Al Candidate | set | ID NO | BS | EC | BL | CA | LM | LP | PP |
| HMPREF9412_4417 | HM | 1 | 64.9 | 59.8 | 69.1 | 56.7 | 51.6 | 51.8 | 50.6 |
| POTG_01507 | HM | 2 | 63.2 | 59.0 | 67.5 | 55.2 | 52.3 | 52.2 | 52.2 |
| HMPREF9374_3716 | HM | 3 | 59.6 | 55.8 | 63.8 | 52.7 | 52.3 | 51.5 | 49.7 |
| DORFOR_01282 | HM | 4 | 57.6 | 51.3 | 58.8 | 52.4 | 52.0 | 51.6 | 51.9 |
| HMPREF0994_04908 | HM | 5 | 56.7 | 52.8 | 57.4 | 49.6 | 48.6 | 48.3 | 48.4 |
| NODE_4061684 | CR | 6 | 55.6 | 49.7 | 56.4 | 51.6 | 48.9 | 48.6 | 47.6 |
| NODE_3664377 | CR | 7 | 52.0 | 50.2 | 56.9 | 51.6 | 51.3 | 49.6 | 50.0 |
| NODE_458803 | CR | 8 | 50.9 | 48.6 | 55.8 | 50.5 | 50.0 | 48.3 | 49.6 |
| NODE_3921064 | CR | 9 | 50.9 | 46.9 | 52.0 | 48.2 | 46.8 | 45.5 | 45.5 |
| NODE_3693095 | CR | 10 | 50.3 | 50.0 | 52.6 | 51.4 | 48.0 | 48.3 | 47.6 |
| DORLON_00938 | HM | 11 | 56.4 | 51.7 | 58.4 | 52.6 | 51.8 | 51.6 | 51.3 |
| HMPREF9469_04726 | HM | 21 | 56.3 | 50.6 | 58.2 | 50.0 | 50.2 | 48.2 | 49.3 |
| HMPREF9467_00216 | HM | 13 | 55.7 | 49.8 | 57.0 | 50.0 | 49.3 | 48.2 | 48.4 |
| RTO_26010 | HM | 14 | 55.3 | 51.3 | 57.6 | 50.2 | 49.6 | 50.3 | 49.3 |
| BRYFOR_08166 | HM | 15 | 55.1 | 49.5 | 57.4 | 51.1 | 49.0 | 47.8 | 48.4 |
| RUMOBE_03031 | HM | 16 | 54.7 | 51.6 | 57.6 | 50.8 | 50.8 | 48.9 | 49.3 |
| NODE_3658038 | CR | 17 | 50.3 | 50.5 | 53.1 | 52.0 | 49.0 | 49.1 | 48.5 |
| NODE_4175755 | CR | 18 | 48.3 | 49.7 | 50.8 | 50.6 | 48.6 | 49.7 | 48.6 |
| NODE_2588280 | CR | 19 | 48.3 | 47.6 | 51.1 | 49.3 | 49.9 | 50.2 | 49.5 |
| NODE_3735508 | CR | 20 | 45.2 | 44.7 | 46.7 | 48.8 | 42.4 | 43.1 | 43.1 |

### Example 6

### Plasmid Constructs and Functional Studies of the Arabinose Isomerase Candidates

Synthetic coding sequences for the twenty arabinose isomerase (AI) candidates identified above were designed and synthesized using codon optimization for expression in S. cerevisiae. They were named araA-1 to araA-20 (SEQ ID NOs:21-40, respectively), corresponding to arabinose isomerase candidates with amino acid sequences of SEQ ID NOs:1-20, respectively. To test these arabinose isomerases, each synthetic coding region was constructed into pSA503 between the last nucleotide of the ADHp fragment and the PacI site in front of the CYC1t fragment, which accurately replaced the BSaraA coding sequence. This resulted in twenty plasmid constructs (from pSA503-1 to pSA503-20) that had sequences identical to pSA503 except for different araA coding regions. All twenty plasmid constructs were transformed into the PXI82-araB strain using the FROZEN-EZ Yeast Transformation II Kit from Zymo Research. Transformants were selected on CM/Gluc/−Ura Plates and named PX182-araBAD50301 to PX182-araBAD50320, corresponding to plasmid constructs pSA503-1 to pSA503-20, respectively. As a control, pSA503 was also transformed into PXI82-araB and selected on a CM/Glud-Ura Plate. Its transformants were named as PXI82-araBAD50300. The constructs and transformants are summarized in Table 4.

TABLE 4
Summary of Twenty Synthetic AraA Nucleotide Sequences and
Their Transformants.

| | | Synthetic | SEQ | Size of | |
|---|---|---|---|---|---|
| Transformant | Plasmid | araA | ID NO | araA | Encoded Al |
| PXI82- | pSA503 | BSaraA | 42 | 1491 nt | BSAI |
| araBAD50300 | | | | | |
| PXI82- | psSA503-1 | araA-1 | 21 | 1488 nt | HMPREF9412_4417 |
| araBAD50301 | | | | | |
| PXI82- | psSA503-2 | araA-2 | 22 | 1488 nt | POTG_01507 |
| araBAD50302 | | | | | |
| PXI82- | psSA503-3 | araA-3 | 23 | 1488 nt | HMPREF9374_3716 |
| araBAD50303 | | | | | |
| PXI82- | psSA503-4 | araA-4 | 24 | 1500 nt | DORFOR_01282 |
| araBAD50304 | | | | | |
| PXI82- | psSA503-5 | araA-5 | 25 | 1497 nt | HMPREF0994_04908 |
| araBAD50305 | | | | | |
| PXI82- | psSA503-6 | araA-6 | 26 | 1497 nt | NODE_4061684 |
| araBAD50306 | | | | | |
| PXI82- | psSA503-7 | araA-7 | 27 | 1434 nt | NODE_3664377 |
| araBAD50307 | | | | | |
| PXI82- | psSA503-8 | araA-8 | 28 | 1434 nt | NODE_458803 |
| araBAD50308 | | | | | |
| PXI82- | psSA503-9 | araA-9 | 29 | 1497 nt | NODE_3921064 |
| araBAD50309 | | | | | |
| PXI82- | psSA503- | araA-10 | 30 | 1464 nt | NODE_3693095 |
| araBAD50310 | 10 | | | | |
| PXI82- | psSA503- | araA-11 | 31 | 1500 nt | DORLON_00938 |
| araBAD50311 | 11 | | | | |
| PXI82- | psSA503- | araA-12 | 32 | 1497 nt | HMPREF9469_04726 |
| araBAD50312 | 12 | | | | |
| PXI82- | psSA503- | araA-13 | 33 | 1497 nt | HMPREF9467_00216 |
| araBAD50313 | 13 | | | | |
| PXI82- | psSA503- | araA-14 | 34 | 1500 nt | RTO_26010 |
| araBAD50314 | 14 | | | | |
| PXI82- | psSA503- | araA-15 | 35 | 1497 nt | BRYFOR_08166 |
| araBAD50315 | 15 | | | | |
| PXI82- | psSA503- | araA-16 | 36 | 1497 nt | RUMOBE_03031 |
| araBAD50316 | 16 | | | | |
| PXI82- | psSA503- | araA-17 | 37 | 1476 nt | NODE_3658038 |
| araBAD50317 | 17 | | | | |
| PXI82- | psSA503- | araA-18 | 38 | 1467 nt | NODE_4175755 |
| araBAD50318 | 18 | | | | |
| PXI82- | psSA503- | araA-19 | 39 | 1467 nt | NODE_2588280 |
| araBAD50319 | 19 | | | | |
| PXI82- | psSA503- | araA-20 | 40 | 1413 nt | NODE_3735508 |
| araBAD50320 | 20 | | | | |

To carry out the functional study for these twenty arabinose isomerases, three transformants were picked as replicas from each of twenty transformations and a control transformation. They were tested for arabinose assimilation and fermentation capacity in the mini-shaking bottle fermentation under micro-aerobic conditions described in Example 4. The fermentation broth used was YPA4 (YP medium containing 40 g/L arabinose). Cultures were grown, and samples were taken and analyzed as described. Fermentation assays for transformants from PX182-araBAD50301 to PX182-araBAD50315 and control transformant PX182-araBAD50300 were carried out for 72 hrs. Fermentations for PX182-araBAD50316 PX182-araBAD50320 were carried out for only 48 hrs because fermentation went fast for those transformants. Therefore, the 48-hr fermentation assays using to PXI82-araBAD50300 were also set up as controls. The assay result for each transformant was an average of fermentation data from three replicas. Ethanol production relative to that of PXI82-araBAD50300 that expressed B. subtilis arabinose isomerase is summarized in Table 5.

TABLE 5
Functional Assays of Twenty AI Candidates

| | | SEQ ID NO | Ethanol Production: % |
|---|---|---|---|
| | Synthetic | of encoded | of PXI82- |
| Transformant | araA | protein | araBAD50300 |
| PXI82-araBAD50300 | BSaraA | 41 | 100.0% |
| PXI82-araBAD50301 | araA-1 | 1 | 0.0% |
| PXI82-araBAD50302 | araA-2 | 2 | 0.0% |
| PXI82-araBAD50303 | araA-3 | 3 | 0.0% |
| PXI82-araBAD50304 | araA-4 | 4 | 39.9% |
| PXI82-araBAD50305 | araA-5 | 5 | 65.5% |
| PXI82-araBAD50306 | araA-6 | 6 | 17.4% |
| PXI82-araBAD50307 | araA-7 | 7 | 110.9% |
| PXI82-araBAD50308 | araA-8 | 8 | 97.7% |
| PXI82-araBAD50309 | araA-9 | 9 | 68.8% |
| PXI82-araBAD50310 | araA-10 | 10 | 105.2% |
| PXI82-araBAD50311 | araA-11 | 11 | 39.6% |
| PXI82-araBAD50312 | araA-12 | 12 | 0.0% |
| PXI82-araBAD50313 | araA-13 | 13 | 43.8% |
| PXI82-araBAD50314 | araA-14 | 14 | 0.0% |
| PXI82-araBAD50315 | araA-15 | 15 | 94.6% |
| PXI82-araBAD50316 | araA-16 | 16 | 0.0% |
| PXI82-araBAD50317 | araA-17 | 17 | 109.9% |
| PXI82-araBAD50318 | araA-18 | 18 | 152.4% |
| PXI82-araBAD50319 | araA-19 | 19 | 139.0% |
| PXI82-araBAD50320 | araA-20 | 20 | 96.1% |

The results showed that six arabinose isomerase candidates, which were constructed into pSA503-1, pSA503-2, pSA503-3, pSA503-12, pSA503-14, and pSA503-16, did not support production of ethanol by S. cerevisiae at all; seven arabinose isomerase candidates, which were constructed into pSA503-4, pSA503-5, pSA503-6, pSA503-9, pSA503-11 and pSA503-13, functioned in S. cerevisiae but were 31.2% to 82.6% less effective in supporting ethanol production than B. subtilis arabinose isomerase. The other eight arabinose isomerase candidates, which were constructed into pSA503-7, pSA503-8, pSA503-10, pSA503-15, pSA503-17, pSA503-18, pSA503-19 and pSA503-20, performed similarly to, or better than, B. subtilis arabinose isomerase for ethanol production. It is interesting to note that all of the candidates in this group originated from the cow rumen dataset except for araA-15, which was from the human microbiome dataset. The best candidate was a cow rumen arabinose isomerase encoded by araA-18. It supported ethanol production up to 152.4% of that supported by the B. subtilis arabinose isomerase.

### Example 7

### In Vitro Activity Assay of the Top Arabinose Isomerase Candidates

Example 6 showed that the top performers in fermentation to produce ethanol included PX182-araBAD50307, PX182-araBAD50308, PX182-araBAD50310, PX182-araBAD50315, PX182-araBAD50317, PX182-araBAD50318, PX182-araBAD50319 and PX182-araBAD50320. To determine whether the arabinose isomerases expressed in these transformants were indeed highly active, the transformants were grown in 25 mL CM/Gluc/−Ura broth (6.7 g/L Sigma yeast nitrogen base without amino acids, 0.77 g/L CLONTECH dropout mix without uracil, 2% glucose) overnight at 32° C. with 200 rpm shaking. At the same time, PX182-araBAD50313 and PX182-araBAD50316 were also grown up as representatives of the groups of arabinose isomerases supporting ethanol production at less than BSara or not at all, respectively. PX182-araBAD50300 was grown as a positive control. To prepare total soluble protein extract, overnight-grown cells with an OD₆₀₀ value of 100 were collected and washed in 10 mL protein extraction buffer (PEB) (10 mM triethanolamine, pH 8.0, 10 mM MgSO₄, 1 mM DTT, 5% glycerol). Cells were resuspended in 1 mL ice-cold PEB with Roche COMPLETE MINI EDTA-Free proteinase inhibitors (product #: 11836170001) and transferred into a tube containing 0.5-mm soda lime glass beads (BioSpec Products, Inc.). Total protein was extracted by beating cells in the tube using a BIO101 FP120 FASTPREP at setting 6 for 30 sec. Beating was repeated six times. Between the beatings, the tube was cooled down on ice for 2 min. Finally, protein extract was obtained by centrifugation and the protein concentration was determined using Coomassie Protein Assay Reagent (Thermo Scientific).

Arabinose isomerase activity in each protein extract was measured by the cysteine-carbazol method (Dische and Borefreund, J. Biol. Chem. 192:583-587). First, a 100-μL assay reaction was assembled to include 10 mM MgSO₄, 10 mM triethanolamine (pH 8.0), 50 mM arabinose, and appropriate amount (2-10 μL) protein extract. The reaction was incubated at 32° C. for 10 min and then stopped by adding 3 mL ice cold 75% H₂SO₄. Ribulose produced in the assay was quantified in a color reaction by adding 100 μL of 2.4% cysteine hydrochloride and 100 μL of 0.12% carbazol ethanolic solution. After incubating at room temperature for 6 min, OD₅₄₀ value was measured using a spectrophotometer. Ribulose concentration was determined by comparing the OD₅₄₀ value with a standard curve of a ribulose color reaction. A unit of arabinose isomerase was defined as the amount of enzyme required to produce one micromole of ribulose in 10 minutes of incubation.

Arabinose isomerase activity of each protein extract was determined by the abovementioned cysteine-carbazol method. For one assay, three replicas were carried out and the result was an average of them. A blank assay without protein extract was set up and deducted from all assay results. Specific activity of a protein extract was calculated based on its activity unit and protein concentration, expressed as unit per milligram of protein per min. FIG. 7 shows arabinose isomerase activities in the protein extracts. The results indicated that (1) protein extract of PX182-araBAD50316 did not have detectable arabinose isomerase activity, which was consistent with its functional assay (Table 5); (2) protein extract of PX182-araBAD50313 presented an activity approximately 2.4-fold higher than that of PXI82-araBAD50300, even though in the functional assay the ethanol productivity of PX182-araBAD50313 was only 43.8% of PXI82-araBAD50300 (Table 5); (3) other protein extracts had activities up to about 20-fold higher than that of PXI82-araBAD50300, but the maximal ethanol productivity was about 152% of PXI82-araBAD50300 (Table 5). Thus ethanol production did not always directly correlate with the in vitro enzyme activity result, perhaps due to the different conditions of the intracellular environment. Good arabinose isomerase candidates should perform well not only in the in vitro activity assay but also in the functional assay. Therefore, both in vitro activity assay and fermentation functional assay confirmed that arabinose isomerase candidates expressed in PX182-araBAD50307, PX182-araBAD50308, PX182-araBAD50310, PX182-araBAD50315, PX182-araBAD50317, PX182-araBAD50318, PX182-araBAD50319, and PX182-araBAD50320 were the enzymes that performed as well or better than B. subtilis arabinose isomerase in S. cerevisiae.

What is claimed is:

1. A recombinant yeast cell comprising an arabinose utilization pathway that comprises a polypeptide having arabinose isomerase activity,
wherein the yeast cell comprises a heterologous polynucleotide encoding said polypeptide,
wherein the polypeptide comprises a motif that is at least 90% identical to SEQ ID NO:67,
to and wherein the position of the motif in the polypeptide corresponds with positions 237-269 of SEQ ID NO:7.

2. The recombinant yeast cell of claim 1, wherein the motif comprises at least seventeen amino acids selected from the group consisting of:
(a) I at position 237;
(b) R or K at position 238;
(c) Y at position 239;
(d) R or K at position 242;
(e) E at position 243;
(f) I at position 245;
(g) A at position 246;
(h) I or M at position 247;
(i) K at position 249;
(j) I or M at position 250;
(k) R or A at position 253;
(l) E or N at position 254;
(m) G at position 255;
(n) A or C at position 256;
(o) F at position 259;
(p) N at position 261;
(q) T at position 262;
(r) Q or E at position 264; and
(s) M at position 269.

3. The recombinant yeast cell of claim 1, wherein the polypeptide comprises a motif that is SEQ ID NO:67.

4. The recombinant yeast cell of claim 1, wherein the polypeptide comprises a motif that is at least 90% identical to SEQ ID NO:66.

5. The recombinant yeast cell of claim 4, wherein the polypeptide comprises a motif that is SEQ ID NO:66.

6. The recombinant yeast cell of claim 1, wherein the polypeptide comprises an amino acid sequence that is at least 85% identical to SEQ ID NO:7, 8, 10, 17, 18, or 19.

7. The recombinant yeast cell of claim 1, further comprising a metabolic pathway that produces a target compound, optionally wherein the target compound is ethanol, butanol, or 1,3-propanediol.

8. A method for producing a yeast cell having arabinose isomerase activity, said method comprising:
(a) providing a yeast cell lacking arabinose isomerase; and
(b) introducing a heterologous polynucleotide into the yeast cell, wherein the heterologous polynucleotide encodes a polypeptide having arabinose isomerase activity, and wherein the polypeptide comprises a motif that is at least 90% identical to SEQ ID NO:67.

9. The method of claim 8, wherein:
(i) the yeast cell of step (a) comprises one or more polynucleotides encoding enzymes, except an arabinose isomerase, of an arabinose utilization pathway, or
(ii) step (b) further comprises introducing, into the yeast cell, one or more polynucleotides encoding enzymes of an arabinose utilization pathway, wherein this further introduction is at the same time of, or after, said introducing the heterologous polynucleotide encoding the polypeptide having arabinose isomerase activity.

10. A method of producing a target compound from arabinose comprising:
(a) providing the recombinant yeast cell of claim 7;
(b) growing the yeast cell of (a) in medium comprising arabinose, wherein the target compound is produced; and
c) optionally isolating the target compound of (b).

11. The method of claim 10, wherein the target compound is ethanol, butanol, or 1,3-propanediol.

12. A recombinant yeast cell comprising an arabinose utilization pathway that comprises a polypeptide having arabinose isomerase activity,
wherein the yeast cell comprises a heterologous polynucleotide encoding said polypeptide,
and wherein the polypeptide comprises an amino acid sequence that is at least 85% identical to SEQ ID NO:15 or 20.

13. A method of producing a target compound from arabinose comprising:
(a) providing the recombinant yeast cell of claim 12;
(b) growing the yeast cell of (a) in medium comprising arabinose, wherein the target compound is produced; and
c) optionally isolating the target compound of (b).


SEQUENCE LISTING (71 sequences)

SEQ ID NO: 1 - protein, 495 aa, unknown
  sequence from Human Microbiome dataset
MNLKPHTFWFVTGSQHLYGPETLEQVAEHSRIVATEFDKDPVFTYPIVFKPIVTTPDEIYKLILEANNDESCAGIMTWMHTFSPAKMWIAGLSQLQKPLLHFHTQFNRDIPWETIDMDFMNLNQSAHGDREYGHIGARLGIARKVVVGHWEDGEVRGSIAGWMRTAAAYAESRRLKVARFGDNMRQVAVTEGDKVEAQIKLGWSVNGYGIGDLVQSMNEVGDEEVKALLNEYAESYSITKEGLSDGPVRDSIAYQARIEIALRRFLEEGGFGAFTTTFEDLHGMKQLPGLAVQRLMESGYGFGGEGDWKTAALTRVLKVLADNKSTSFMEDYTYHFEPGNHMILGSHMLEVCPTIALDKPTLEVHPLGIGGKGDPARLVFNGQDGPAVNASLIDLGHRFRLLVNVVDGVKVEQPMPKLPVARVLWKPQPSLRESAEAWILAGGAHHTVLSYAMTAEHLSDWAEMTGIEAVVIDKDTTIPRFKNELRWSEAAYRLR

SEQ ID NO: 2 - protein, 495 aa, unknown
  sequence from Human Microbiome dataset
MKLKPHSFWFVTGSQHLYGPETLEEVAGHSRIIAEQLDKDPAIGFPVVFKPIVTTPDEIYKLILAANGDETCAGIITWMHTFSPAKMWIAGLSQLQKPLLHFHTQFNRDIPWETIDMDFMNLNQSAHGDREYGHIGARLGINRKIVVGHWEDEEVRASLAGWMRTAVAYAESRQLKVARFGDNMREVAVTEGDKVEAQIKFGWSVNGYGVGDLVQVLNEVTDAEAEALLKEYAEQYTITQAGLSSGPIRDSIAYQAKLEIAMKRFLEQGGFGAFTTTFEDLHGLKQLPGLAVQRLMEAGYGFGGEGDWKTAALTRVLKVLANNKSTSFMEDYTYHFEPGNHMILGAHMLEVCPTIAATKPTIEVHPLGIGGKADPARMVFDGQAGPAVNASLVDLGHRFRLLVNVVDGVKVEKPMPKLPVARVLWKPQPSLRESAEAWILAGGAHHTVLSYAITAENLSDWAEMVGIEAVIIDKDTSVPRFKNELRWSDAAYRLR

SEQ ID NO: 3 - protein, 495 aa, unknown
  sequence from Human Microbiome dataset
MQRTPYEFWFVTGSQHLYGSEALAEVSSHSRQITQAFNEADSISFPIVVKPVVKTPEEILQLCMEANSDENCAGLITWMHTFSPGKMWIGGLSQLHKPLLHFHTQFHREIPWDRIDMDFMNLHQSAHGDREFGFIATRLGILRKEVVGHWRDEAVQKRLSDWMRTAIACLEGKKLKVARFGDNMRRVAVTEGDKVEAQIQFGWSINGYGVGDLVQRITDISDTAVHQLFREYQERYDFPPEAREAGPIRDSILEQARIELGLKLFLREGGYSAFTTTFEDLHGLKQLPGLAVQRLMSEGYGFGAEGDWRTAGLLRMMKIMADNEGTSFMEDYTYHLEPGNEMILGAHMLEVCPTIAAQRPGIRVHPLSIGGKADPARLVFDGRPGPALNVSLIDLGNRFRLLINKVDAVHPKSAMPHLPVARVLWKPRPSLHDSAEAWMYAGGAHHTVFSYHVTTEQLLDWAEWVDMEALVIDEQTSLSSFRRQLKWNDAYYRIR

SEQ ID NO: 4 - protein, 499 aa, unknown
  sequence from Human Microbiome dataset
MLKTKNYQFWFCTGSQDLYGDECLAHVAEHAKKIVEALNASGNLPYEVVWKPTLITNELIRRTFNEANTDENCAGVITWMHTFSPAKSWILGLQEFRKPLLHLHTQFNREIPYDTIDMDFMNENQSAHGDREFGHIFSRLHMNRKVVVGYWADEDVQKQIGSWMRTAVGVVESSHIRVMRIADNMRNVAVTEGDKVEAQIKFGWEVDAYPVNEAVEAVNAVSQADIDTLVEEYYDKYEILLEGRDEKEFRRHVAVQAGIEIGLERFLEENNYQAIVTHFGDLGGFKQLPGLAMQRLMEKGYGFGAEGDWKTAAMVRLMKIMTGGMKDAKGTSFMEDYTYNLVPGKEGILEAHMLEVCPTIADGKISIKEQPLSMGDREDPARLVFTAKEGPAIAASLIDLGDRFRLLINEVECKKTEKPMPKLPVATAFWTPKPNLKIGAQSWILAGGAHHTAFSYDLSAEQMGDWAEAMGIEAVYIDADTTIRQLKNELRWNELAYRR

SEQ ID NO: 5 - protein, 498 aa, unknown
  sequence from Human Microbiome dataset
MKTGRDYKFWFCTGSQDLYGEECLRKVAEHSAKIVEGLNASGRLPFEVVLKPTLIDPATIRRTLNEANEDGECAGVITWMHTFSPAKMWILGLKEYRKPLCHLHTQFNEEIPYDTIDMDFMNENQSAHGDREFGHMVSRMGMERKIIVGHWANAEVQEKIGSWMRTAIGIMESSHIRVCRIGDNMNNVAVTEGDKVEAEVKFGWEIDHYCVNDAVEYVNAVSEGDVNALVEEYYSKYQILLEGRDPEEFRAHVAAQAKIEIGLEKFLEDGDYHAIVTHFGMLGGLQQLPGLAIQRLMEKGYGFGGEGDWKTAAMVRLMKIMAAGVPGAKGTSFMEDYTYNLVPGKEGILQAHMLEVCPSIAEGPISIKVQPLSMGNREDPARLVFTSKTGPAVATSLVDLGNRFRLIINAVDCKKCEKEMPKLPVATAFWTPQPDLATGAQAWILAGGAHHTAFSYDLTVDQMVDWAAAMGIESVVIDKDTTIRNFKNELRWNSIYYR

SEQ ID NO: 6 - protein, 498 aa, unknown
  sequence from cow rumen metagenome dataset
MIQTKAYKFWFCTGSQDLYGDEVLRHVADHSKEIVEELNKSGILPYEVVWKPVLITNQLIRQTFNEANADDSCAGVITWMHTFSPAKSWILGLQEFRKPLLHLHTQYNEEIPYDTIDMDFMNENQAAHGDREYGHIVSRMGIERKVIAGYWKDNEVRSRIASWMRTAVGVMESSHIRVMRVADNMRNVAVTEGDKVEAQIKFGWEVDTYPVNEIADSVATVSASDVNALLDEYYDKYEIILDGRDPDEFKKHVAVQAQIELGFERFLEEKNYQAIVTHFGDLGALGQLPGLAIQRLMEKGYGFGAEGDWKVAAMVRLMKIMTSGMKDAKGTSMLEDYTYNLVRGKEGILEAHMLEICPTIADGPISIRVKPLSMGDREDPARLVFTSKEGKGVATSLIDLGNRFRLIINEVECKKTEKPMPNLPVATAYWTPYPDLYTGAEAWILAGGAHHTAFSYDLTSGQMADWAEMMGIEAVIIDKNTTIPAFKKELKLGDVFYR

SEQ ID NO: 7 - protein, 477 aa, unknown
  sequence from cow rumen metagenome dataset
MKFWFVTGSQFLYGEETLRQVEEDSKKIVDGLRLPFPVEYKLTVKTESEIERIVKEANYDDECAGIITFCHTFSPSKMWINGLALLQKPWLHFHTQFNETIPNEAIDMDYMNLHQSAHGDREHGFIGARLRVPRAVVAGYWKDPAVQAKIGEWQRAAVGVMFSRSLKIVRFGDNMREVAVTEGDKIEAQLRLGWQVNTFAVGDLVEYMDAVTDAEIDALMKEYAELYEFSEADTDTIRYQAREEIAIEKILVREGAKAFSNTFEDLHGMKQLPGLATQHLMHKGYGFGAEGDWKTAGMTAIVKAMYPDGNTSFMEDYTYDYERQLILGSHMLEVCPSIAADRPRIEVHKLGIGGKDAPARIVFEGRAGSAKVLSLIDIGGRFRLIQQDIECEKPFQSMPNLPVARTMWRPAPSFLEGLECWIIAGGAHHTVLSYDITDETVRDFARIMGIELVVINKDTTKEKLERDIMIGDVIYGR

SEQ ID NO: 8 - protein, 477 aa, unknown
  sequence from cow rumen metagenome dataset
MKFWFITGSQFLYGEETIRQVEEDSKKIVDGLKLPFPVEYKLTVKKESEIERIVKEANFDDECAGIITFCHTFSPSKMWINGLAILQKPWLHFHTQFNETIPNEAIDMAYMNLHQSAHGDREHGFIGARLRMPRAVVAGYWKDPEVQAKIAEWQRAAVGVMFSKSLKIVRFGDNMREVAVTEGDKIEAQLKLGWQVNTFAVGDLVEYMNAVTDAEIDVLMKEYAELYDYDKADEETIRYQAREEIAIEKILVREGAKAFSNTFEDLHGMQQLPGLATQHLMHKGYGFGAEGDWKTAGMTAIVKAMYPDGNTSFMEDYTYDYERKLILGSHMLEVCPSIAADRPRIEVHPLGIGGKEPPARIVFEGKAGSAKVLSLIDIGGRLRLIQQDIECEKPFQSMPNLPVARTMWRPAPSFLEGLECWIIAGGAHHTVLSYDISDETVRDFARIMGIELVVINKDTTKEKLERDIMIGDMIYGR

SEQ ID NO: 9 - protein, 498 aa, unknown
  sequence from cow rumen metagenome dataset
MSEMKKYQFWFCTGSQDLYGDECLAHVAAHSKEMVEGLNKSGVLPFEIVWKPTLITNELIRKTFNEANNDPNCAGVITWMHTFSPAKSWILGLQEFRKPLLHLHTQYNEEIPYATMDMDFMNENQAAHGDREYAHILSRMRIERKVVVGFWKDSEVQKKIASWMRTAIGIMESSHIRVCRVADNMRNVAVTEGDKVEAQLKFGWEIDAYPVNEIAEAVAAVSASDTNALVDEYYSKYDICLEGRDPEEFKKHVAVQAQIEIGFERFLKEKNYQAIVTHFGDLGALKQLPGLAIQRLMEKGYGFGAEGDWKVAAMVRLMKIMSAGMKDAKGSSMLEDYTYNLVKGKEGIIQAHMLEICPSISDGPIQIKCQPLSMGDREDPARLVFQSKTGAGIATSLIDLGNRFRLIIQDVECKKVEKPLPKLPTAINFWTPQPDFYTGTEAWLLAGGAHHTAFSYDITAEQMGDWAAAMGIEAVFIDKNTNIRDFKKDLMLGEVFYR

SEQ ID NO: 10 - protein, 487 aa, unknown
  sequence from cow rumen metagenome dataset
MQREFWFIVGSQFLYGQDVLDTVDARAREMAAELSKVLPYPLVYKVTAKTNKEIADTVKEANYRDEVMGIVTWCHTFSPSKMWINGLVNLQKPYCHLATQYNRELPNEEIDIDFMNLNQAAHGDREHGFIAARLRMPRKVIAGYWQDEKVHKRLSDWMKAAVGVDVSKHMKVMRFGDNMREVAVTEGDKVETQIKLGWQVNTWAVGDLVKEMNNVTEAEIDALFAEYEAQYDIATDNLAAIRYQAKEEIAMKKMLDREGCKAFSNTFQDLYGMEQLPGLASQHLMAQGYGYGGEGDWKVSAMTAILKAMGENGNGASAFMEDYTYHLVEGQEYSLGAHMLEVCPSLAADKPRIETHHLGIGMNEKDPARLVFEGKAGKGIVTSLIDMGGRMRLIVQDIEAVKPILPMPNLPVARVMWRAMPDLTTGVECWITAGGAHHTVLSFDVTPAMLRDWARMMDIEFVYITKDTTPEELEEELLIKDLVWKLK

SEQ ID NO: 11 - protein, 499 aa, unknown
  sequence from Human Microbiome dataset
MLKTKNYQFWFCTGSQDLYGDECLAHVAEHSKIIVDALNKSGNLPYEVVWKPTMITNEVIRKTFNEANTDENCAGVITWMHTFSPAKSWILGLQEYRKPLLHLHTQFNREIPYDTIDMDFMNENQAAHGDREYGHIFSRLNMERKVVAGYWEDEDVQKQIGSWMRTAVGVVESSHVRVMRVADNMRNVAVTEGDKVEAQIKFGWEVDAYPVNEVVEAVNAVSQADIDTLVEEYYDKYDILLEGRDEKEFREHVAVQAGIELGFERFLDENNYQAVVTHFGDLGGLKQLPGLAMQRLMEKGYGFGAEGDWKTAAMVRVMKIMTQGMKDAKGTSFMEDYTYNLVSGKEGVLEAHMLEVCPTIADGKISIKEQPLSMGNREDPARLVFTSKTGPAIATSLIDLGDRFRLIINDVDCKKTEKPMPKLPVATAFWTPQPNLKVGTEAWILAGGAHHTAFSYDLTAEQMGDWAACMGIEAVYIDKDTTIRQFKNELLWNSVAYRK

SEQ ID NO: 12 - protein, 498 aa, unknown
  sequence from Human Microbiome dataset
MTGVKNYKFWFCTGSQDLYGEECLAHVAEHSRIIVESLNRSGILPYEVVWKPTLITNELIRRTFNEANADEECAGVITWMHTFSPAKSWILGLQEFRKPLMHFHTQFNREIPYDTIDMDFMNENQSAHGDREYGHMVTRMGIERKVIVGHWSDEKVVGRIAGWMRTAVGIMESSHVRVVRFADNMRNVAVTEGDKVEAQVKFGWEVDAYPVNELCQYVKAVPKGDITALVDEYYSKYTILLEGRDPEEFKRHVAVQAQIEAGLERFLVEKDYHAIVTHFGDLGELQQLPGLAIQRLMEKGYGFGGEGDWKTAAMVRLMKIMAQGVKNAKGTSFMEDYTYNLVPGKEGILEAHMLEVCPSIADGEISIKVNPLSMGDREDPARLVFTSKTGHGIATSLVDLGTRFRLIINDVECRKTEKAMPKLPVATAFWTPEPSLATGAEAWILAGGAHHTAFSYDLTAEQMGDWAESMGIEVVYIDKDTTIRGLKNEMRWNGAVYR

SEQ ID NO: 13 - protein, 498 aa, unknown
  sequence from Human Microbiome dataset
MIAVKNYKFWFCTGSQDLYGDECLAHVAEHSGIIVDSLNKSGILPYEVVLKPTLITNELIRRTFNEANADEECAGVITWMHTFSPAKSWILGLQEYRKPLMHFHTQFNQEIPYDSIDMDFMNENQSAHGDREYGHMVTRMGIERKVIVGHWRDEKVVGRIAAWMRTAVGIMESSHVRVARFADNMRNVAVTEGDKVEAQMKFGWEVDAYPVNELAEYVKAVPKGDITALVDEYYSKYTILLEGRDPEEFKRHVAVQAQIEAGLEKFLLEKDYHAIVTHFGDLGELQQLPGLAIQRLMEKGYGFGAEGDWKTAAMVRLMKIMTQGMKDAKGTSFMEDYTYNLVPGKEGILEAHMLEVCPTIADGEISIKACPLSMGDREDPARLVFTSKTGHGIAASLVDLGTRFRLIINDVECKKTEKPMPKLPVATAFWTPEPNLATGAESWILAGGAHHTAFSYDLTAEQMGDWADAMGIETVYIDKDTTIRGLKNELRWNAAAYR

SEQ ID NO: 14 - protein, 499 aa, unknown
  sequence from Human Microbiome dataset
MLKKKEYKFWFCTGSQDLYGDECLAHVAEHAKIIVEKLNESGVLPYEVVWKPTLITNELIRKTFNEANIDDECAGVITWMHTFSPAKSWILGLQEFRKPLLHLHTQFNMEIPYDTIDMDFMNENQSAHGGREFGHIFTRLGIERKVVVGHWSDEKVQEKIASWMRTAVGVIESSHVRVMRVADNMRNVAVTEGDKVEAQIKFGWEVDAYPVNEIAESVDAVSAADVNTLVEEYYDKYEILLEGRDPEEFRKHVAVQAQIELGFERFLEEKNYQAIVTHFGDLGVLKQLPGLAIQRLMQKGYGFGAEGDWKTAAMVRIMKIMTEGMKDAKGTSMLEDYTYNFVPGKEGILQAHMLEICPSIADGPISIKVNPLSMGDREDPARLVFTSKEGKGIATSLIDLGDRFRLIINTVDCKKNEKPMPKLPVATNFWTPEPDLATGAEAWILCGGAHHTAFSYDITAEQMGDWAAMMGIEAVYIDKDTTIRNLKNELRWNELAFRK

SEQ ID NO: 15 - protein, 498 aa, unknown
  sequence from Human Microbiome dataset
MKAAKDYKFWFCTGSQDLYGDECLAHVAEHSRIIVDALNKSGVLPYEIVWKPTLITNELIRKTFNEANADENCAGVITWMHTFSPAKSWILGLQEFRKPLLHFHTQFNREIPYDTIDMDFMNENQAAHGDREYGHIVSRMGIERKIIVGYWEDRDVQEKIASWMLTAIGIMESSHIRVCRIADNMRNVAVTEGDKVEAQIKFGWEIDAYPVNEIAEYVAAVPQGEINALVEEYYSKYDIILEGRDPQEFREHVAVQAGIEIGFEKFLEEKNYQAIVTHFGDLGSLKQLPGLAIQRLMEKGYGFGGEGDWKTAAMVRLMKIMTAGVKNPKGTSFMEDYTYNLVPGKEGVLEAHMLEVCPSVADGPIGIKVCPLSMGDREDPARLVYTSKTGPAIATSLIDLGNRFRLIINEVECKKVEKPMPKLPVATAFWTPYPDLKTGAEAWILAGGAHHTAFSYDLTAEQMGDWAAAMGIEAVYIDKDTTIRNFKRDLQLGNIVYR

SEQ ID NO: 16 - protein, 498 aa, unknown
  sequence from Human Microbiome dataset
MVTGRNYKFWFCTGSQDLYGDECLRKVAEHSRIIVEELNKSGVLPFELVWKPTLITNELIRKTFNEANADDECAGVITWMHTFSPAKSWILGLKEYRKPLCHLHTQFNQEIPYDTIDMDFMNENQSAHGGREYGHIVTRMGIERKVIVGHWADKKVQERLASWMRTAVGIMESSHIRVCRVADNMRNVAVTEGDKVEAQIKFGWEVDAYPVNEVCDYVKDVSKGDIDVLVEEYYNKYDILFEGRDPEEFKRHVAVQAAIEIGFERFLEEKNYQAVVTHFGDLGGLQQLPGLAMQRLMEKGYGFGAEGDWKTAAMVRLMKIMTAGVKDAKGTSFMEDYTYNLVPGKEGILQSHMLEVCPTIADGKIGIKVCPLSMGDREDPARLFTSKTGPAVATSLVDLGDRFRLIINDVDCKKVEKPMPKLPVGSAFWTPQPDLATGAEAWILAGGAHHTAFSYDLTAEQMGDWAAAMGIEAVYIDKDTTIRNFKNELRWNEVAFRK

SEQ ID NO: 17 - protein, 491 aa, unknown
  sequence from cow rumen metagenome dataset
MEDIMKREFWFIVGSQFLYGQDVLDTVDARAKEMAAELSKVLPYPLVYKVTAKTNKEITDVIKEANYRDECAGIVTWCHTFSPSKMWINGLANLQKPYCHLATQYNKEIPNDEIDMDFMNLNQAAHGDREHGFIAARLRLPRKVIAGFWQDEKIHKRLSDWMRAAVGVAVSKKMKVMRFGDNMREVAVTEGDKVEVQTKLGWQVNTWAVGDLVKEMGKVTEAEIDALVAEYEANYDIATDNTAAIRYQAREEIAMKKMLDREGCRAFTNTFQDLYGMEQLPGLASQHLMAQGYGYGGEGDWKVSAMTAILKAMGENGNGASGFMEDYTYHLVEGQEYSLGAHMLEVCPSLAADKPRIETHHLGIGMNEKDPARLVFEGKAGKGIVVSLIDMGGRLRLIVQDIEAVKPILPMPNLPVARVMWRAMPDLTTGVECWITAGGAHHTVLSYDVTAEQMRDWARMMDIEFVHITKDTTPEKLEEELLVKDLVWKLK

SEQ ID NO: 18 - protein, 488 aa, unknown
  sequence from cow rumen metagenome dataset
MSKEFWFVVGSQDLYGEEVLKIVAERAAEMAAWLSEKLPYPLIYKVTAMSSNQITSVMKEANFDDNCLGVVTWCHTFSPSKMWLTGLDLLQKPWCHFATQYNLEIPNEEIDMDFMNLNQAAHGDREHGFIGARLRKARKVVAGYWKDEKVIARLAEFQKVAVGVDASKHMKVMRFGDNMRDVAVTEGDKVEVQKKLGWEVNTWAVGDLVKEMNAVTDEEVEALFNEYKASYDINTDNIYAIKYQAREEIAIKKMMDRNGCKAFSNTFQDLYGMEQLPGLASQHLMSLGYGYGGEGDWKVSAMTAILKAMGENGNGASAFMEDYTYHLVKGHEYSLGAHMLEVCPSLAADKPRIETHHLGIGMNEKDPARLVFEGKEGRGIVASLIDMGGRLRLIVQDIEAVKPIMPMPNLPVARVMWRALPDLTDGVECWITAGGAHHTVLSYDVTPEMMRDFAKFMDIEFVHIDKDTTVEKLEDELMVKDLVWKMKG

SEQ ID NO: 19 - protein, 488 aa, unknown
  sequence from cow rumen metagenome dataset
MGNKKNFWFVVGSQFLYGNEVLETVAARAQEMAEKMSKSLPYELKFKGIVKTWDEATQYAKEANFDDNCCGVITWCHTFSPSKMWIEAFRLLQKPLLHFATQYNRYIPDKEIDMDFMNLNQAAHGDREHGFIIARMRLQQKIVTGFWEDQPVLDEIGTWMRAAVAYDFSRNLRVMRFGDNMREVAVTEGDKVEAQIKFGWQVNTWPVGKLVEEIGKVTEEEVDELLKVYTDTYELATDDIETIRYQAREEIAMKKMMTAEGANAFVNTFQDLIGMKQLPGIASQHLMAQGYGYGAEGDWKLSALVSIVKKMTEGMTGGTSFMEDYTYHLDPNAEYALGAHMLEVCPSIAADKPRIEVHPLGIGDREDPARLVFEGHEGDAVVVTLIDMGERFRMLVQDIHCVKPIYEMPNLPVARVMWEGKPSLNEGLKMWLMAGGAHHSVLSYDATPEMLKDLARMMDIEFVHITADSKPEEFEKDLFFADLAWKLK

SEQ ID NO: 20 - protein, 470 aa, unknown
  sequence from cow rumen metagenome dataset
MKKIYFITGSQDLYGEDVLKTVAKDSQEMVNFLDEQVGERAEIEFLGVVRDSEICLDFILKANFDKECIGIITWMHTFSPAKMWIRGLKVLHKPMLHLHTQYNEKLPYDSIDMDFMNLNQAAHGDREFGFIAARMNIKQHVLSGYYKNKDFIEGVKQYIDVCLSIDAAKYLRVAMFGSNMRDVAVTDGDRVQSEIDFGWNVNYYGIGDLVDIINKVKDEEIDAQFEEYKKRYTINTTNIEAIKEQAKYEVALKKFIKKENVQAFTDNFQDLHGLKQLPGLAVQDLMQEGISFGPEGDYKTPALLATLLPMTKYRKGATGFIEDYTYDLIEGKEIELGSHMLEVPPSFATSKPEIQVRPLSIGDKAAPARLVFDSIEGEGLQITMVDMGTHFRIIAAKIQLVKQPAPMPKLPVARIMWKHIPNFKISTEAWMLYGGGHHSVITTALTIEDIKLFAKLTGTELCVIDENTKI

SEQ ID NO: 21 - DNA, 1488 bp, unknown
  Sequence from the human microbiome dataset
atgaacttgaagccacacaccttctggttcgtcactggttcccaacacttgtacggtccagaaactttggaacaagtcgctgaacactccagaatcgtcgctactgaatttgacaaggacccagttttcacctacccaatcgtcttcaagccaatcgttaccactccagatgaaatctacaagttgatcttggaagctaacaacgacgaatcctgtgctggtatcatgacttggatgcacaccttctctccagctaagatgtggatcgctggtttgtcccaattgcaaaagccattgttgcacttccacactcaattcaacagagatatcccttgggaaaccatcgacatggatttcatgaacttgaaccaatctgctcacggtgacagagaatacggtcacatcggtgctagattgggtatcgctagaaaggttgtcgttggtcactgggaagacggtgaagtcagaggttctatcgctggttggatgagaaccgctgctgcttacgctgaatccagaagattgaaggtcgctagattcggtgacaacatgagacaagtcgctgttaccgaaggtgacaaggttgaagctcaaatcaagttgggttggtctgtcaacggttacggtatcggtgacttggttcaatccatgaacgaagtcggtgacgaagaagttaaggctttgttgaacgaatacgctgaatcttactccatcactaaggaaggtttgtctgacggtccagtcagagattccatcgcttaccaagctagaatcgaaatcgctttgagaagattcttggaagaaggtggtttcggtgctttcaccactaccttcgaagacttgcacggtatgaagcaattgccaggtttggctgttcaaagattgatggaatctggttacggtttcggtggtgaaggtgactggaagactgctgctttgaccagagtcttgaaggttttggctgacaacaagtctacttccttcatggaagattacacctaccacttcgaaccaggtaaccacatgatcttgggttctcacatgttggaagtctgtccaactatcgctttggacaagccaaccttggaagttcacccattgggtatcggtggtaaaggtgacccagctagattggtcttcaacggtcaagatggtccagctgttaacgcttctttgatcgacttgggtcacagattcagattgttggtcaacgtcgttgacggtgtcaaggttgaacaaccaatgccaaagttgccagtcgctagagttttgtggaagccacaaccatctttgagagaatctgctgaagcctggatcttggctggtggtgctcaccacactgttttgtcttacgctatgaccgctgaacacttgtccgactgggctgaaatgaccggtatcgaagctgtcgttatcgacaaggatactaccatcccaagattcaagaacgaattgagatggtctgaagctgcttacagattgagatga

SEQ ID NO: 22 - DNA, 1488 bp, unknown
  Sequence from the human microbiome dataset
atgaagttgaagccacactctttctggttcgttactggttcccaacacttgtacggtccagaaaccttggaagaagtcgctggtcactccagaatcatcgctgaacaattggacaaggacccagctatcggtttcccagttgtcttcaagccaatcgttaccactccagacgaaatctacaagttgatcttggctgctaacggtgacgaaacttgtgctggtatcatcacttggatgcacaccttctctccagctaagatgtggatcgctggtttgtcccaattgcaaaagccattgttgcacttccacactcaattcaacagagacatcccttgggaaaccatcgacatggatttcatgaacttgaaccaatctgctcacggtgacagagaatacggtcacatcggtgctagattgggtatcaacagaaagatcgttgtcggtcactgggaagacgaagaagtcagagcttctttggctggttggatgagaactgctgttgcttacgctgaatccagacaattgaaggtcgctagattcggtgacaacatgagagaagttgctgtcaccgaaggtgacaaggttgaagctcaaatcaagttcggttggtctgttaacggttacggtgtcggtgacttggttcaagtcttgaacgaagtcaccgatgctgaagctgaagccttgttgaaggaatacgctgaacaatacactatcacccaagctggtttgtcttccggtccaatcagagactccatcgcttaccaagccaagttggaaatcgctatgaagagattcttggaacaaggtggtttcggtgctttcaccactaccttcgaagacttgcacggtttgaagcaattgccaggtttggctgttcaaagattgatggaagctggttacggtttcggtggtgaaggtgactggaagactgctgctttgaccagagttttgaaggtcttggctaacaacaagtctacttccttcatggaagactacacctaccacttcgaaccaggtaaccacatgatcttgggtgctcacatgttggaagtttgtccaactatcgctgctactaagccaaccatcgaagtccacccattgggtatcggtggtaaagctgacccagctagaatggttttcgatggtcaagctggtccagctgttaacgcttctttggttgacttgggtcacagattcagattgttggtcaacgttgtcgatggtgttaaggtcgaaaagccaatgccaaagttgccagttgctagagtcttgtggaagccacaaccatctttgagagaatctgctgaagcctggatcttggctggtggtgctcaccacactgttttgtcttacgctatcaccgctgaaaacttgtccgactgggctgaaatggttggtatcgaagctgtcatcatcgacaaggatacctctgtcccaagattcaagaacgaattgagatggtccgacgctgcttacagattgagatga

SEQ ID NO: 23 - DNA, 1488 bp, unknown
  Sequence from the human microbiome dataset
atgcaaagaactccatacgaattctggttcgtcactggttcccaacacttgtacggttccgaagctttggctgaagtctcctcccactccagacaaatcactcaagccttcaacgaagctgactctatctccttcccaatcgttgtcaagccagttgtcaagaccccagaagaaatcttgcaattgtgtatggaagctaactctgacgaaaactgtgctggtttgatcacttggatgcacaccttctctccaggtaaaatgtggatcggtggtttgtcccaattgcacaagccattgttgcacttccacactcaattccacagagaaatcccttgggacagaatcgacatggatttcatgaacttgcaccaatctgctcacggtgacagagaatttggtttcatcgctaccagattgggtatcttgagaaaggaagttgtcggtcactggagagacgaagctgttcaaaagagattgtctgattggatgagaactgctatcgcttgtttggaaggtaaaaagttgaaggttgctagattcggtgacaacatgagaagagttgctgtcaccgaaggtgacaaggtcgaagctcaaatccaattcggttggtctatcaacggttacggtgttggtgacttggtccaaagaatcactgacatctccgataccgctgttcaccaattgttcagagaataccaagaaagatacgacttcccaccagaagctagagaagctggtccaatcagagattctatcttggaacaagctagaatcgaattgggtttgaagttgttcttgagagaaggtggttactccgctttcaccactaccttcgaagacttgcacggtttgaagcaattgccaggtttggctgtccaaagattgatgtctgaaggttacggtttcggtgctgaaggtgactggagaactgctggtttgttgagaatgatgaagatcatggctgacaacgaaggtacttccttcatggaagattacacctaccacttggaaccaggtaacgaaatgatcttgggtgctcacatgttggaagtttgtccaaccatcgctgctcaaagaccaggtatcagagtccacccattgtctatcggtggtaaagctgacccagctagattggttttcgatggtagaccaggtccagctttgaacgtctctttgatcgacttgggtaacagattcagattgttgatcaacaaggttgatgctgtccacccaaagtccgctatgccacacttgccagttgctagagtcttgtggaagccaagaccatctttgcacgactctgctgaagcctggatgtacgctggtggtgctcaccacactgttttctcttaccacgtcactaccgaacaattgttggactgggctgaatgggttgatatggaagccttggttatcgacgaacaaacctccttgtcttccttcagaagacaattgaagtggaacgacgcttactacagaatcagatga

SEQ ID NO: 24 - DNA, 1500 bp, unknown
  Sequence from the human microbiome dataset
atgttgaagactaagaactaccaattctggttctgtactggttcccaagatttgtacggtgacgaatgtttggctcacgttgctgaacacgctaagaagatcgttgaagccttgaacgcttctggtaacttgccatacgaagttgtctggaagccaaccttgatcactaacgaattgatcagaagaaccttcaacgaagctaacactgacgaaaactgtgctggtgtcatcacctggatgcacactttctctccagctaagtcttggatcttgggtttgcaagagttcagaaagccattgttgcacttgcacacccaattcaacagagaaatcccatacgacactatcgacatggatttcatgaacgaaaaccaatctgctcacggtgacagagaatttggtcacatcttctccagattgcacatgaacagaaaggttgtcgttggttactgggctgacgaagatgttcaaaagcaaatcggttcttggatgagaaccgctgttggtgtcgttgaatcttcccacatcagagtcatgagaatcgctgacaacatgagaaacgtcgctgttactgaaggtgacaaggttgaagctcaaatcaagttcggttgggaagttgacgcttacccagtcaacgaagctgtcgaagctgttaacgctgtctcccaagctgacatcgataccttggtcgaagaatactacgacaagtacgaaatcttgttggaaggtagagatgaaaaggagttcagaagacacgtcgctgttcaagctggtatcgaaatcggtttggaaagattcttggaagaaaacaactaccaagctatcgttactcacttcggtgacttgggtggtttcaagcaattgccaggtttggctatgcaaagattgatggaaaagggttacggtttcggtgctgaaggtgactggaagaccgctgctatggtcagattgatgaagatcatgactggtggtatgaaggacgctaagggtacttctttcatggaagattacacttacaacttggttccaggtaaagaaggtatcttggaagctcacatgttggaagtctgtccaaccatcgctgacggtaaaatctctatcaaggaacaaccattgtctatgggtgacagagaagatccagctagattggttttcactgctaaggaaggtccagctatcgctgcttctttgatcgacttgggtgacagattcagattgttgatcaacgaagttgaatgtaagaagaccgaaaagccaatgccaaagttgccagtcgctaccgctttctggactccaaagccaaacttgaagatcggtgctcaatcctggatcttggctggtggtgctcaccacactgctttctcttacgacttgtccgctgaacaaatgggtgactgggctgaagctatgggtatcgaagctgtctacatcgacgctgataccactatcagacaattgaagaacgaattgagatggaacgaattggcttacagaagatga

SEQ ID NO: 25 - DNA, 1497 bp, unknown
  Sequence from the human microbiome dataset
atgaagactggtagagattacaagttctggttctgtactggttcccaagatttgtacggtgaagaatgtttgagaaaggttgctgaacactccgctaagatcgtcgaaggtttgaacgcttctggtagattgccattcgaagttgtcttgaagccaaccttgatcgatccagctaccatcagaagaactttgaacgaagctaacgaagacggtgaatgtgctggtgttatcacctggatgcacactttctccccagctaagatgtggatcttgggtttgaaggaatacagaaagccattgtgtcacttgcacacccaattcaacgaagaaatcccatacgatactatcgacatggatttcatgaacgaaaaccaatccgctcacggtgacagagaatttggtcacatggtttccagaatgggtatggaaagaaagatcatcgtcggtcactgggctaacgctgaagttcaagaaaagatcggttcttggatgagaaccgctatcggtatcatggaatcttcccacatcagagtctgtagaatcggtgacaacatgaacaacgttgctgtcactgaaggtgacaaggtcgaagctgaagtcaagttcggttgggaaatcgatcactactgtgttaacgacgctgttgaatacgtcaacgctgtttccgaaggtgacgtcaacgctttggttgaagaatactactctaagtaccaaatcttgttggaaggtagagacccagaagagttcagagctcacgtcgctgctcaagctaagatcgaaatcggtttggaaaagttcttggaagacggtgactaccacgctatcgttacccacttcggtatgttgggtggtttgcaacaattgccaggtttggctatccaaagattgatggaaaagggttacggtttcggtggtgaaggtgactggaagactgctgctatggtcagattgatgaagatcatggctgctggtgttccaggtgctaagggtacttctttcatggaagactacacttacaacttggtcccaggtaaagaaggtatcttgcaagctcacatgttggaagtctgtccatctatcgctgaaggtccaatctccatcaaggttcaaccattgtctatgggtaacagagaagacccagctagattggttttcacctccaagactggtccagctgtcgctacctctttggttgatttgggtaacagattcagattgatcatcaacgctgttgactgtaagaagtgtgaaaaggaaatgccaaagttgccagttgctaccgctttctggactccacaaccagacttggctactggtgctcaagcctggatcttggctggtggtgctcaccacaccgctttctcctacgacttgactgtcgatcaaatggttgactgggctgctgctatgggtatcgaatctgttgtcatcgacaaggataccactatcagaaacttcaagaacgaattgagatggaactctatctactacagatga

SEQ ID NO: 26 - DNA, 1497 bp, unknown
  Sequence from the  cow rumen metagenome dataset
atgatccaaactaaggcttacaagttctggttctgtactggttcccaagatttgtacggtgacgaagttttgagacacgttgctgatcactctaaggaaatcgttgaagaattgaacaagtccggtatcttgccatacgaagttgtctggaagccagtcttgatcaccaaccaattgatcagacaaactttcaacgaagctaacgctgacgattcttgtgctggtgttatcacctggatgcacactttctctccagctaagtcttggatcttgggtttgcaagagttcagaaagccattgttgcacttgcacacccaatacaacgaagaaatcccatacgatactatcgacatggatttcatgaacgaaaaccaagctgctcacggtgacagagaatacggtcacatcgtttccagaatgggtatcgaaagaaaggtcatcgctggttactggaaggacaacgaagttagatccagaatcgcttcctggatgagaaccgctgttggtgtcatggaatcttcccacatcagagttatgagagtcgctgataacatgagaaacgttgctgtcactgaaggtgacaaggttgaagctcaaatcaagttcggttgggaagttgacacctacccagtcaacgaaatcgctgattctgttgctactgtctctgcttccgacgtcaacgctttgttggacgaatactacgataagtacgaaatcatcttggacggtagagacccagatgagttcaagaagcacgttgctgtccaagctcaaatcgaattgggtttcgaaagattcttggaagaaaagaactaccaagctatcgttacccacttcggtgacttgggtgctttgggtcaattgccaggtttggctatccaaagattgatggaaaagggttacggtttcggtgctgaaggtgactggaaggttgctgctatggtcagattgatgaagatcatgacctctggtatgaaggatgctaagggtacttccatgttggaagactacacttacaacttggttagaggtaaagaaggtatcttggaagctcacatgttggaaatctgtccaactatcgctgacggtccaatctctatcagagtcaagccattgtctatgggtgacagagaagatccagctagattggttttcacctctaaggaaggtaaaggtgtcgctacttccttgatcgacttgggtaacagattcagattgatcatcaacgaagttgaatgtaagaagaccgaaaagccaatgccaaacttgccagtcgctaccgcttactggactccatacccagacttgtacactggtgctgaagcctggatcttggctggtggtgctcaccacaccgctttctcttacgacttgacttccggtcaaatggctgattgggctgaaatgatgggtatcgaagctgttatcatcgataagaacaccactatcccagctttcaagaaggaattgaagttgggtgacgtcttctacagatga

SEQ ID NO: 27 - DNA, 1434 bp, unknown
  Sequence from the  cow rumen metagenome dataset
atgaagttctggttcgttactggttcccaattcttgtacggtgaagaaaccttgagacaagttgaagaagactctaagaagatcgttgacggtttgagattgccattcccagttgaatacaagttgaccgtcaagactgaatctgaaatcgaaagaatcgttaaggaagctaactacgacgatgaatgtgctggtatcatcaccttctgtcacactttctctccatccaagatgtggatcaacggtttggctttgttgcaaaagccttggttgcacttccacacccaattcaacgaaactatcccaaacgaagctatcgacatggattacatgaacttgcaccaatccgctcacggtgacagagaacacggtttcatcggtgctagattgagagttccaagagctgttgtcgctggttactggaaggacccagctgtccaagctaagatcggtgaatggcaaagagctgctgttggtgtcatgttctccagatccttgaagatcgtcagattcggtgacaacatgagagaagttgctgtcaccgaaggtgacaagatcgaagctcaattgagattgggttggcaagttaacaccttcgctgttggtgacttggtcgaatacatggacgctgtcactgatgctgaaatcgacgctttgatgaaggaatacgctgaattgtacgaattttctgaagctgacaccgatactatcagataccaagctagagaagaaatcgctatcgaaaagattttggttagagaaggtgctaaggctttctccaacaccttcgaagacttgcacggtatgaagcaattgccaggtttggctactcaacacttgatgcacaagggttacggtttcggtgctgaaggtgactggaagaccgctggtatgactgctatcgtcaaggctatgtacccagacggtaacacctctttcatggaagattacacttacgactacgaaagacaattgatcttgggttctcacatgttggaagtttgtccatccatcgctgctgatagaccaagaatcgaagtccacaagttgggtatcggtggtaaagacgctccagctagaatcgttttcgaaggtagagctggttctgctaaggtcttgtccttgatcgatatcggtggtagattcagattgatccaacaagacatcgaatgtgaaaagccattccaatctatgccaaacttgccagttgctagaaccatgtggagaccagctccatccttcttggaaggtttggaatgttggatcatcgctggtggtgctcaccacactgttttgtcttacgacatcaccgatgaaactgtcagagatttcgctagaatcatgggtatcgaattggttgtcatcaacaaggacaccactaaggaaaagttggaaagagatatcatgatcggtgacgtcatctacggtagatga

SEQ ID NO: 28 - DNA, 1434 bp, unknown
  Sequence from the  cow rumen metagenome dataset
atgaagttctggttcatcactggttcccaattcttgtacggtgaagaaactatcagacaagtcgaagaagattccaagaagatcgtcgacggtttgaagttgccattcccagttgaatacaagttgaccgtcaagaaggaatctgaaatcgaaagaatcgttaaggaagctaacttcgacgatgaatgtgctggtatcatcaccttctgtcacactttctctccatccaagatgtggatcaacggtttggctatcttgcaaaagccttggttgcacttccacacccaattcaacgaaactatcccaaacgaagctatcgacatggcttacatgaacttgcaccaatctgctcacggtgacagagaacacggtttcatcggtgctagattgagaatgccaagagctgttgtcgctggttactggaaggacccagaagttcaagctaagatcgctgaatggcaaagagctgctgttggtgtcatgttctctaagtccttgaagatcgtcagattcggtgacaacatgagagaagttgctgtcaccgaaggtgacaagatcgaagctcaattgaagttgggttggcaagtcaacaccttcgctgttggtgacttggtcgaatacatgaacgctgttactgacgctgaaatcgatgtcttgatgaaggaatacgctgaattgtacgactacgataaggctgacgaagaaactatcagataccaagctagagaagaaatcgctatcgaaaagattttggttagagaaggtgctaaggctttctctaacaccttcgaagacttgcacggtatgcaacaattgccaggtttggctactcaacacttgatgcacaagggttacggtttcggtgctgaaggtgactggaagaccgctggtatgactgctatcgtcaaggctatgtacccagacggtaacacctccttcatggaagactacacttacgattacgaaagaaagttgatcttgggttctcacatgttggaagtttgtccatccatcgctgctgacagaccaagaatcgaagtccacccattgggtatcggtggtaaagaaccaccagctagaatcgttttcgaaggtaaagctggttctgctaaggtcttgtccttgatcgacatcggtggtagattgagattgatccaacaagatatcgaatgtgaaaagccattccaatctatgccaaacttgccagttgctagaactatgtggagaccagctccatccttcttggaaggtttggaatgttggatcatcgctggtggtgctcaccacaccgttttgtcttacgacatctccgatgaaactgtcagagacttcgctagaatcatgggtatcgaattggttgtcatcaacaaggataccactaaggaaaagttggaaagagacatcatgatcggtgacatgatctacggtagatga

SEQ ID NO: 29 - DNA, 1497 bp, unknown
  Sequence from the  cow rumen metagenome dataset
atgtccgaaatgaagaagtaccaattctggttctgtactggttcccaagatttgtacggtgacgaatgtttggctcacgttgctgctcactctaaggaaatggtcgaaggtttgaacaagtccggtgtcttgccattcgaaatcgtttggaagccaaccttgatcactaacgaattgatcagaaagaccttcaacgaagctaacaacgacccaaactgtgctggtgttatcacctggatgcacactttctctccagctaagtcttggatcttgggtttgcaagagttcagaaagccattgttgcacttgcacacccaatacaacgaagaaatcccatacgctactatggacatggatttcatgaacgaaaaccaagctgctcacggtgacagagaatacgctcacatcttgtccagaatgagaatcgaaagaaaggttgtcgttggtttctggaaggattctgaagtccaaaagaagatcgcttcctggatgagaaccgctatcggtatcatggaatcttcccacatcagagtctgtagagttgctgacaacatgagaaacgtcgctgttactgaaggtgacaaggtcgaagctcaattgaagttcggttgggaaatcgacgcttacccagttaacgaaatcgctgaagctgtcgctgctgtttctgcttccgacaccaacgctttggtcgatgaatactactctaagtacgacatctgtttggaaggtagagatccagaagagttcaagaagcacgtcgctgttcaagctcaaatcgaaatcggtttcgaaagattcttgaaggaaaagaactaccaagctatcgttactcacttcggtgacttgggtgctttgaagcaattgccaggtttggctatccaaagattgatggaaaagggttacggtttcggtgctgaaggtgactggaaggtcgctgctatggttagattgatgaagatcatgtctgctggtatgaaggacgctaagggttcttccatgttggaagattacacctacaacttggtcaagggtaaagaaggtatcatccaagctcacatgttggaaatctgtccatctatctccgacggtccaatccaaatcaagtgtcaaccattgtctatgggtgacagagaagatccagctagattggttttccaatctaagaccggtgctggtatcgctacttccttgatcgacttgggtaacagattcagattgatcatccaagatgtcgaatgtaagaaggttgaaaagccattgccaaagttgccaaccgctatcaacttctggactccacaaccagacttctacaccggtactgaagcctggttgttggctggtggtgctcaccacaccgctttctcttacgacatcactgctgaacaaatgggtgactgggctgctgctatgggtatcgaagctgtcttcatcgacaagaacactaacatcagagacttcaagaaggatttgatgttgggtgaagttttctacagatga

SEQ ID NO: 30 - DNA, 1464 bp, unknown
  Sequence from the  cow rumen metagenome dataset
atgcaaagagaattctggttcatcgtcggttcccaattcttgtacggtcaagatgttttggacactgttgatgctagagctagagaaatggctgctgaattgtctaaggtcttgccatacccattggtctacaaggttaccgctaagactaacaaggaaatcgctgacactgttaaggaagctaactacagagatgaagtcatgggtatcgttacctggtgtcacactttctctccatccaagatgtggatcaacggtttggtcaacttgcaaaagccatactgtcacttggctacccaatacaacagagaattgccaaacgaagaaatcgacatcgatttcatgaacttgaaccaagctgctcacggtgacagagaacacggtttcatcgctgctagattgagaatgccaagaaaggtcatcgctggttactggcaagacgaaaaggttcacaagagattgtctgattggatgaaggctgctgttggtgttgacgtttccaagcacatgaaggtcatgagattcggtgacaacatgagagaagtcgctgttaccgaaggtgacaaggtcgaaactcaaatcaagttgggttggcaagttaacacttgggctgtcggtgacttggttaaggaaatgaacaacgttaccgaagctgaaatcgacgctttgttcgctgaatacgaagctcaatacgacatcgctactgataacttggctgctatcagataccaagctaaggaagaaatcgctatgaagaagatgttggatagagaaggttgtaaggctttctctaacaccttccaagacttgtacggtatggaacaattgccaggtttggcttcccaacacttgatggctcaaggttacggttacggtggtgaaggtgactggaaggtctctgctatgactgctatcttgaaggctatgggtgaaaacggtaacggtgcttccgctttcatggaagactacacctaccacttggtcgaaggtcaagaatactctttgggtgctcacatgttggaagtttgtccatccttggctgctgacaagccaagaatcgaaactcaccacttgggtatcggtatgaacgaaaaggacccagctagattggtcttcgaaggtaaagctggtaaaggtatcgttacctctttgatcgatatgggtggtagaatgagattgatcgtccaagacatcgaagctgttaagccaatcttgccaatgccaaacttgccagtcgctagagttatgtggagagctatgccagacttgaccactggtgttgaatgttggatcaccgctggtggtgctcaccacactgtcttgtctttcgacgttaccccagctatgttgagagactgggctagaatgatggatatcgaatttgtctacatcactaaggataccactccagaagaattggaagaagaattgttgatcaaggacttggtttggaagttgaagtga

SEQ ID NO: 31 - DNA, 1500 bp, unknown
  Sequence from the human microbiome dataset
atgttgaagactaagaactaccaattctggttctgtactggttcccaagatttgtacggtgacgaatgtttggctcacgttgctgaacactctaagatcatcgttgacgctttgaacaagtccggtaacttgccatacgaagttgtctggaagccaaccatgatcactaacgaagttatcagaaagaccttcaacgaagctaacactgacgaaaactgtgctggtgtcatcacctggatgcacactttctctccagctaagtcttggatcttgggtttgcaagaatacagaaagccattgttgcacttgcacacccaattcaacagagaaatcccatacgacactatcgacatggatttcatgaacgaaaaccaagctgctcacggtgacagagaatacggtcacatcttctccagattgaacatggaaagaaaggttgtcgctggttactgggaagacgaagatgttcaaaagcaaatcggttcctggatgagaaccgctgtcggtgttgtcgaatcttcccacgttagagtcatgagagttgctgacaacatgagaaacgttgctgtcactgaaggtgacaaggtcgaagctcaaatcaagttcggttgggaagttgacgcttacccagtcaacgaagttgtcgaagctgttaacgctgtctctcaagctgacatcgataccttggttgaagaatactacgacaagtacgatatcttgttggaaggtagagacgaaaaggagttcagagaacacgttgctgtccaagctggtatcgaattgggtttcgaaagattcttggacgaaaacaactaccaagctgttgtcactcacttcggtgacttgggtggtttgaagcaattgccaggtttggctatgcaaagattgatggaaaagggttacggtttcggtgctgaaggtgactggaagaccgctgctatggttagagtcatgaagatcatgactcaaggtatgaaggacgctaagggtacttctttcatggaagattacacttacaacttggtttccggtaaagaaggtgtcttggaagctcacatgttggaagtctgtccaaccatcgctgacggtaaaatctctatcaaggaacaaccattgtctatgggtaacagagaagacccagctagattggttttcacctctaagactggtccagctatcgctacctccttgatcgacttgggtgacagattcagattgatcatcaacgacgtcgattgtaagaagactgaaaagccaatgccaaagttgccagttgctaccgctttctggactccacaaccaaacttgaaggtcggtactgaagcctggatcttggctggtggtgctcaccacaccgctttctcttacgacttgactgctgaacaaatgggtgactgggctgcttgtatgggtatcgaagctgtttacatcgacaaggataccactatcagacaattcaagaacgaattgttgtggaactctgtcgcttacagaaagtaa

SEQ ID NO: 32 - DNA, 1497 bp, unknown
  Sequence from the human microbiome dataset
atgactggtgttaagaactacaagttctggttctgtactggttcccaagatttgtacggtgaagaatgtttggctcacgtcgctgaacactccagaatcatcgttgaatctttgaacagatccggtatcttgccatacgaagttgtctggaagccaaccttgatcactaacgaattgatcagaagaaccttcaacgaagctaacgctgacgaagaatgtgctggtgtcatcacctggatgcacactttctctccagctaagtcttggatcttgggtttgcaagagttcagaaagccattgatgcacttccacacccaattcaacagagaaatcccatacgacactatcgacatggatttcatgaacgaaaaccaatccgctcacggtgacagagaatacggtcacatggttactagaatgggtatcgaaagaaaggttatcgtcggtcactggtctgacgaaaaggttgtcggtagaatcgctggttggatgagaaccgctgttggtatcatggaatcttcccacgtcagagttgtcagattcgctgacaacatgagaaacgttgctgtcactgaaggtgacaaggttgaagctcaagtcaagttcggttgggaagttgacgcttacccagtcaacgaattgtgtcaatacgttaaggctgtcccaaagggtgacatcaccgctttggtcgatgaatactactccaagtacactatcttgttggaaggtagagacccagaagagttcaagagacacgttgctgtccaagctcaaatcgaagctggtttggaaagattcttggttgaaaaggactaccacgctatcgtcacccacttcggtgacttgggtgaattgcaacaattgccaggtttggctatccaaagattgatggaaaagggttacggtttcggtggtgaaggtgactggaagactgctgctatggttagattgatgaagatcatggctcaaggtgtcaagaacgctaagggtacttctttcatggaagactacacttacaacttggttccaggtaaagaaggtatcttggaagctcacatgttggaagtttgtccatctatcgctgacggtgaaatctccatcaaggtcaacccattgtctatgggtgacagagaagatccagctagattggttttcacctccaagactggtcacggtatcgctacctctttggttgacttgggtactagattcagattgatcatcaacgatgtcgaatgtagaaagaccgaaaaggctatgccaaagttgccagtcgctaccgctttctggactccagaaccatctttggctactggtgctgaagcctggatcttggctggtggtgctcaccacaccgctttctcctacgacttgactgctgaacaaatgggtgactgggctgaatctatgggtatcgaagttgtctacatcgacaaggataccactatcagaggtttgaagaacgaaatgagatggaacggtgctgtctacagataa

SEQ ID NO: 33 - DNA, 1497 bp, unknown
  Sequence from the human microbiome dataset
atgatcgctgttaagaactacaagttctggttctgtactggttcccaagatttgtacggtgacgaatgtttggctcacgttgctgaacactctggtatcatcgttgactctttgaacaagtccggtatcttgccatacgaagttgtcttgaagccaaccttgatcactaacgaattgatcagaagaaccttcaacgaagctaacgctgacgaagaatgtgctggtgtcatcacctggatgcacactttctctccagctaagtcttggatcttgggtttgcaagaatacagaaagccattgatgcacttccacacccaattcaaccaagaaatcccatacgactctatcgacatggatttcatgaacgaaaaccaatccgctcacggtgacagagaatacggtcacatggttactagaatgggtatcgaaagaaaggttatcgtcggtcactggagagacgaaaaggttgtcggtagaatcgctgcttggatgagaaccgctgttggtatcatggaatcttcccacgttagagtcgctagattcgctgacaacatgagaaacgttgctgtcactgaaggtgacaaggtcgaagctcaaatgaagttcggttgggaagttgacgcttacccagtcaacgaattggctgaatacgttaaggctgtcccaaagggtgacatcaccgctttggtcgatgaatactactctaagtacactatcttgttggaaggtagagacccagaagagttcaagagacacgttgctgtccaagctcaaatcgaagctggtttggaaaagttcttgttggaaaaggactaccacgctatcgttacccacttcggtgacttgggtgaattgcaacaattgccaggtttggctatccaaagattgatggaaaagggttacggtttcggtgctgaaggtgactggaagaccgctgctatggtcagattgatgaagatcatgactcaaggtatgaaggacgctaagggtacttctttcatggaagattacacttacaacttggttccaggtaaagaaggtatcttggaagctcacatgttggaagtctgtccaactatcgctgacggtgaaatctctatcaaggcttgtccattgtctatgggtgacagagaagatccagctagattggttttcacctctaagactggtcacggtatcgctgcttccttggttgacttgggtactagattcagattgatcatcaacgatgtcgaatgtaagaagactgaaaagccaatgccaaagttgccagtcgctaccgctttctggactccagaaccaaacttggctaccggtgctgaatcttggatcttggctggtggtgctcaccacaccgctttctcctacgacttgactgctgaacaaatgggtgactgggctgatgctatgggtatcgaaactgtttacatcgacaaggataccactatcagaggtttgaagaacgaattgagatggaacgctgctgcttacagataa

SEQ ID NO: 34 - DNA, 1500 bp, unknown
  Sequence from the human microbiome dataset
atgttgaagaagaaggaatacaagttctggttctgtactggttcccaagatttgtacggtgacgaatgtttggctcacgttgctgaacacgctaagatcatcgtcgaaaagttgaacgaatccggtgttttgccatacgaagttgtctggaagccaaccttgatcactaacgaattgatcagaaagaccttcaacgaagctaacatcgacgatgaatgtgctggtgtcatcacctggatgcacactttctctccagctaagtcttggatcttgggtttgcaagagttcagaaagccattgttgcacttgcacacccaattcaacatggaaatcccatacgacactatcgacatggatttcatgaacgaaaaccaatctgctcacggtggtagagaatttggtcacatcttcactagattgggtatcgaaagaaaggttgtcgttggtcactggtccgacgaaaaggttcaagaaaagatcgcttcttggatgagaaccgctgtcggtgttatcgaatcttcccacgtcagagttatgagagtcgctgacaacatgagaaacgtcgctgttactgaaggtgacaaggttgaagctcaaatcaagttcggttgggaagttgacgcttacccagttaacgaaatcgctgaatctgttgacgctgtttccgctgctgatgtcaacaccttggttgaagaatactacgacaagtacgaaatcttgttggaaggtagagatccagaagagttcagaaagcacgtcgctgttcaagctcaaatcgaattgggtttcgaaagattcttggaagaaaagaactaccaagctatcgtcactcacttcggtgacttgggtgttttgaagcaattgccaggtttggctatccaaagattgatgcaaaagggttacggtttcggtgctgaaggtgactggaagaccgctgctatggtcagaatcatgaagatcatgactgaaggtatgaaggacgctaagggtacttctatgttggaagattacacttacaacttcgttccaggtaaagaaggtatcttgcaagctcacatgttggaaatctgtccatctatcgctgacggtccaatctccatcaaggtcaacccattgtctatgggtgacagagaagatccagctagattggttttcacctccaaggaaggtaaaggtatcgctacttctttgatcgacttgggtgacagattcagattgatcatcaacaccgttgactgtaagaagaacgaaaagccaatgccaaagttgccagttgctaccaacttctggactccagaaccagacttggctactggtgctgaagcctggatcttgtgtggtggtgctcaccacaccgctttctcttacgacatcactgctgaacaaatgggtgactgggctgctatgatgggtatcgaagctgtctacatcgacaaggataccactatcagaaacttgaagaacgaattgagatggaacgaattggctttcagaaagtaa

SEQ ID NO: 35 - DNA, 1497 bp, unknown
  Sequence from the human microbiome dataset
atgaaggctgctaaggattacaagttctggttctgtactggttctcaagatttgtacggtgacgaatgtttggctcacgttgctgaacactccagaatcatcgttgacgctttgaacaagtccggtgttttgccatacgaaatcgtctggaagccaaccttgatcactaacgaattgatcagaaagaccttcaacgaagctaacgctgacgaaaactgtgctggtgtcatcacctggatgcacactttctctccagctaagtcttggatcttgggtttgcaagagttcagaaagccattgttgcacttccacacccaattcaacagagaaatcccatacgacactatcgacatggatttcatgaacgaaaaccaagctgctcacggtgacagagaatacggtcacatcgtttccagaatgggtatcgaaagaaagatcatcgttggttactgggaagacagagatgtccaagaaaagatcgcttcctggatgttgaccgctatcggtatcatggaatcttcccacatcagagtctgtagaatcgctgacaacatgagaaacgttgctgtcactgaaggtgacaaggttgaagctcaaatcaagttcggttgggaaatcgacgcttacccagtcaacgaaatcgctgaatacgttgctgctgtcccacaaggtgaaatcaacgctttggttgaagaatactactctaagtacgacatcatcttggaaggtagagatccacaagagttcagagaacacgttgctgtccaagctggtatcgaaatcggtttcgaaaagttcttggaagaaaagaactaccaagctatcgtcactcacttcggtgacttgggttctttgaagcaattgccaggtttggctatccaaagattgatggaaaagggttacggtttcggtggtgaaggtgactggaagaccgctgctatggttagattgatgaagatcatgactgctggtgtcaagaacccaaagggtacttctttcatggaagactacacttacaacttggttccaggtaaagaaggtgtcttggaagctcacatgttggaagtttgtccatctgtcgctgatggtccaatcggtatcaaggtttgtccattgtctatgggtgacagagaagatccagctagattggtctacacctctaagactggtccagctatcgctacctccttgatcgacttgggtaacagattcagattgatcatcaacgaagttgaatgtaagaaggtcgaaaagccaatgccaaagttgccagttgctaccgctttctggactccatacccagacttgaagactggtgctgaagcctggatcttggctggtggtgctcaccacaccgctttctcttacgacttgactgctgaacaaatgggtgactgggctgctgctatgggtatcgaagctgtttacatcgacaaggataccactatcagaaacttcaagagagacttgcaattgggtaacatcgtctacagataa

SEQ ID NO: 36 - DNA, 1497 bp, unknown
  Sequence from the human microbiome dataset
atggttactggtagaaactacaagttctggttctgtactggttcccaagatttgtacggtgacgaatgtttgagaaaggttgctgaacactccagaatcatcgttgaagaattgaacaagtccggtgttttgccattcgaattggtctggaagccaaccttgatcactaacgaattgatcagaaagaccttcaacgaagctaacgctgacgatgaatgtgctggtgtcatcacctggatgcacactttctctccagctaagtcttggatcttgggtttgaaggaatacagaaagccattgtgtcacttgcacacccaattcaaccaagaaatcccatacgacactatcgacatggatttcatgaacgaaaaccaatctgctcacggtggtagagaatacggtcacatcgttactagaatgggtatcgaaagaaaggttatcgtcggtcactgggctgacaagaaggttcaagaaagattggcttcctggatgagaaccgctgtcggtatcatggaatcttcccacatcagagtttgtagagtcgctgacaacatgagaaacgttgctgtcactgaaggtgacaaggttgaagctcaaatcaagttcggttgggaagttgacgcttacccagttaacgaagtctgtgactacgttaaggatgtctctaagggtgacatcgatgttttggtcgaagaatactacaacaagtacgacatcttgttcgaaggtagagatccagaagagttcaagagacacgttgctgtccaagctgctatcgaaatcggtttcgaaagattcttggaagaaaagaactaccaagctgttgtcacccacttcggtgacttgggtggtttgcaacaattgccaggtttggctatgcaaagattgatggaaaagggttacggtttcggtgctgaaggtgactggaagaccgctgctatggttagattgatgaagatcatgactgctggtgtcaaggacgctaagggtacttctttcatggaagattacacttacaacttggttccaggtaaagaaggtatcttgcaatcccacatgttggaagtttgtccaaccatcgctgacggtaaaatcggtatcaaggtctgtccattgtctatgggtgacagagaagatccagctagattgttcacctctaagactggtccagctgttgctacttccttggttgacttgggtgacagattcagattgatcatcaacgacgttgattgtaagaaggtcgaaaagccaatgccaaagttgccagtcggttctgctttctggaccccacaaccagacttggctactggtgctgaagcctggatcttggctggtggtgctcaccacaccgctttctcctacgacttgactgctgaacaaatgggtgactgggctgctgctatgggtatcgaagctgtttacatcgacaaggataccactatcagaaacttcaagaacgaattgagatggaacgaagtcgctttcagaaagtaa

SEQ ID NO: 37 - DNA, 1476 bp, unknown
  Sequence from the cow rumen metagenome dataset
atggaagacatcatgaagagagaattttggttcatcgttggttcccaattcttgtacggtcaagacgttttggacactgttgacgctagagctaaggaaatggctgctgaattgtctaaggtcttgccatacccattggtctacaaggttaccgctaagactaacaaggaaatcactgacgtcatcaaggaagctaactacagagatgaatgtgctggtatcgttacctggtgtcacactttctctccatccaagatgtggatcaacggtttggctaacttgcaaaagccatactgtcacttggctacccaatacaacaaggaaatcccaaacgacgaaatcgacatggatttcatgaacttgaaccaagctgctcacggtgacagagaacacggtttcatcgctgctagattgagattgccaagaaaggttatcgctggtttctggcaagacgaaaagatccacaagagattgtctgattggatgagagctgctgttggtgtcgctgtttccaagaagatgaaggtcatgagattcggtgacaacatgagagaagtcgctgttactgaaggtgacaaggtcgaagttcaaactaagttgggttggcaagttaacacctgggctgtcggtgacttggttaaggaaatgggtaaagtcaccgaagctgaaatcgatgctttggttgctgaatacgaagctaactacgacatcgctaccgataacactgctgctatcagataccaagctagagaagaaatcgctatgaagaagatgttggacagagaaggttgtagagctttcaccaacactttccaagatttgtacggtatggaacaattgccaggtttggcttctcaacacttgatggctcaaggttacggttacggtggtgaaggtgactggaaggtctctgctatgactgctatcttgaaggctatgggtgaaaacggtaacggtgcttccggtttcatggaagactacacctaccacttggtcgaaggtcaagaatactctttgggtgctcacatgttggaagtttgtccatccttggctgctgacaagccaagaatcgaaactcaccacttgggtatcggtatgaacgaaaaggacccagctagattggttttcgaaggtaaagctggtaaaggtatcgttgtctctttgatcgacatgggtggtagattgagattgatcgtccaagatatcgaagctgttaagccaatcttgccaatgccaaacttgccagtcgctagagttatgtggagagctatgccagacttgaccactggtgtcgaatgttggatcaccgctggtggtgctcaccacactgtcttgtcctacgacgttaccgctgaacaaatgagagactgggctagaatgatggatatcgaatttgttcacatcaccaaggacactaccccagaaaagttggaagaagaattgttggttaaggatttggtttggaagttgaagtaa

SEQ ID NO: 38 - DNA, 1467 bp, unknown
  Sequence from the cow rumen metagenome dataset
atgtctaaggaattttggttcgtcgtcggttcccaagatttgtacggtgaagaagttttgaagatcgtcgctgaaagagctgctgaaatggctgcttggttgtctgaaaagttgccatacccattgatctacaaggtcactgctatgtcttccaaccaaatcacctccgttatgaaggaagctaacttcgacgataactgtttgggtgttgtcacctggtgtcacactttctctccatccaagatgtggttgactggtttggacttgttgcaaaagccttggtgtcacttcgctacccaatacaacttggaaatcccaaacgaagaaatcgacatggatttcatgaacttgaaccaagctgctcacggtgacagagaacacggtttcatcggtgctagattgagaaaggctagaaaggttgtcgctggttactggaaggacgaaaaggtcatcgctagattggctgaatttcaaaaggttgctgtcggtgttgacgcttctaagcacatgaaggttatgagattcggtgacaacatgagagatgtcgctgttactgaaggtgacaaggtcgaagttcaaaagaagttgggttgggaagtcaacacttgggctgtcggtgacttggttaaggaaatgaacgctgtcaccgatgaagaagttgaagccttgttcaacgaatacaaggcttcttacgacatcaacactgataacatctacgctatcaagtaccaagctagagaagaaatcgctatcaagaagatgatggacagaaacggttgtaaggctttctccaacaccttccaagatttgtacggtatggaacaattgccaggtttggcttctcaacacttgatgtccttgggttacggttacggtggtgaaggtgactggaaggtttctgctatgactgctatcttgaaggctatgggtgaaaacggtaacggtgcttccgctttcatggaagactacacctaccacttggtcaagggtcacgaatactctttgggtgctcacatgttggaagtttgtccatccttggctgctgacaagccaagaatcgaaacccaccacttgggtatcggtatgaacgaaaaggacccagctagattggtcttcgaaggtaaagaaggtagaggtatcgttgcttctttgatcgacatgggtggtagattgagattgatcgtccaagatatcgaagctgttaagccaatcatgccaatgccaaacttgccagtcgctagagttatgtggagagctttgccagacttgactgatggtgtcgaatgttggatcaccgctggtggtgctcaccacactgtcttgtcttacgacgttaccccagaaatgatgagagacttcgctaagttcatggatatcgaatttgttcacatcgacaaggataccaccgttgaaaagttggaagatgaattgatggttaaggatttggtttggaagatgaagggttaa

SEQ ID NO: 39 - DNA, 1467 bp, unknown
  Sequence from the cow rumen metagenome dataset
atgggtaacaagaagaacttctggttcgtcgtcggttctcaattcttgtacggtaacgaagttttggaaactgtcgctgctagagctcaagaaatggctgaaaagatgtctaagtccttgccatacgaattgaagttcaagggtatcgtcaagacctgggacgaagctactcaatacgctaaggaagctaacttcgacgataactgttgtggtgttatcacctggtgtcacactttctctccatccaagatgtggatcgaagccttcagattgttgcaaaagccattgttgcacttcgctacccaatacaacagatacatcccagacaaggaaatcgacatggatttcatgaacttgaaccaagctgctcacggtgacagagaacacggtttcatcatcgctagaatgagattgcaacaaaagatcgtcaccggtttctgggaagaccaaccagttttggatgaaatcggtacttggatgagagctgctgtcgcttacgacttctccagaaacttgagagttatgagattcggtgacaacatgagagaagtcgctgttactgaaggtgacaaggtcgaagctcaaatcaagttcggttggcaagttaacacctggccagtcggtaaattggttgaagaaatcggtaaagtcactgaagaagaagttgacgaattgttgaaggtctacaccgatacttacgaattggctaccgacgatatcgaaactatcagataccaagctagagaagaaatcgctatgaagaagatgatgaccgctgaaggtgctaacgctttcgttaacactttccaagacttgatcggtatgaagcaattgccaggtatcgcttctcaacacttgatggctcaaggttacggttacggtgctgaaggtgactggaagttgtctgctttggtctccatcgttaagaagatgaccgaaggtatgaccggtggtacttctttcatggaagactacacttaccacttggacccaaacgctgaatacgctttgggtgctcacatgttggaagtctgtccatccatcgctgctgacaagccaagaatcgaagttcacccattgggtatcggtgacagagaagatccagctagattggtcttcgaaggtcacgaaggtgacgctgttgtcgttaccttgatcgatatgggtgaaagattcagaatgttggtccaagacatccactgtgttaagccaatctacgaaatgccaaacttgccagtcgctagagttatgtgggaaggtaaaccatctttgaacgaaggtttgaagatgtggttgatggctggtggtgctcaccactctgtcttgtcctacgacgctaccccagaaatgttgaaggacttggctagaatgatggatatcgaatttgttcacatcactgctgactccaagccagaagaatttgaaaaggacttgttcttcgctgatttggcttggaagttgaagtaa

SEQ ID NO: 40 - DNA, 1413 bp, unknown
  Sequence from the cow rumen metagenome dataset
atgaagaagatttacttcatcactggttcccaagacttgtacggtgaagacgttttgaagactgtcgctaaggactcccaagaaatggttaacttcttggacgaacaagtcggtgaaagagctgaaatcgaatttttgggtgttgtcagagactctgaaatctgtttggatttcatcttgaaggctaacttcgacaaggaatgtatcggtatcatcacctggatgcacactttctccccagctaagatgtggatcagaggtttgaaggttttgcacaagccaatgttgcacttgcacacccaatacaacgaaaagttgccatacgactctatcgacatggatttcatgaacttgaaccaagctgctcacggtgacagagaatttggtttcatcgctgctagaatgaacatcaagcaacacgttttgtccggttactacaagaacaaggacttcatcgaaggtgttaagcaatacatcgacgtctgtttgtctatcgatgctgctaagtacttgagagtcgctatgttcggttccaacatgagagacgttgctgtcactgacggtgacagagttcaatctgaaatcgacttcggttggaacgtcaactactacggtatcggtgacttggttgatatcatcaacaaggtcaaggacgaagaaatcgatgctcaattcgaagaatacaagaagagatacaccatcaacaccactaacatcgaagctatcaaggaacaagccaagtacgaagttgctttgaagaagttcatcaagaaggaaaacgtccaagccttcaccgacaacttccaagatttgcacggtttgaagcaattgccaggtttggctgttcaagacttgatgcaagaaggtatctctttcggtccagaaggtgactacaagaccccagctttgttggctaccttgttgccaatgactaagtacagaaagggtgctaccggtttcatcgaagactacacttacgatttgatcgaaggtaaagaaatcgaattgggttctcacatgttggaagttccaccatctttcgctacttccaagccagaaatccaagtcagaccattgtctatcggtgacaaggctgctccagctagattggttttcgattccatcgaaggtgaaggtttgcaaatcactatggttgacatgggtactcacttcagaatcatcgctgctaagatccaattggttaagcaaccagctccaatgccaaagttgccagtcgctagaatcatgtggaagcacatcccaaacttcaagatttctaccgaagcctggatgttgtacggtggtggtcaccactccgtcatcaccactgctttgactatcgaagacatcaagttgttcgctaagttgactggtactgaattgtgtgttatcgatgaaaacactaagatttaa

SEQ ID NO: 41 - protein, 496 aa, Bacillus subtilis
MLQTKDYEFWFVTGSQHLYGEETLELVDQHAKSICEGLSGVSSRYKITHKPVVTSSETIRQLLREAEYSETCAGIITWMHTFSPAKMWIEGLSSYQKPLMHLHTQYNRDIPWGTIDMDFMNSNQSAHGDREYGYINSRMGLSRKVVAGYWDDEEVKKEISQWMDTAAALNESRHIKVARFGDNMRHVAVTDGDKVGAHIQFGWQVDGYGIGDLVEVMNRITDDEVDTLYAEYDRLYVISEETKRDEAKVASIKEQAKIELGLTTFLEQGGYSAFTTSFEVLHGMKQLPGLAVQRLMEKGYGFAGEGDWKTAALVRMMKIMSQGKRTSFMEDYTYHFEPGNEMILGSHMLEVCPTVALDQPKIEVHPLSIGGKEDPARFVFNGISGSAIQASLVDIGGRFRLVLNEVNGQEIEKDMPNLPVARVLWKPEPSLKTAAEAWILAGGAHHTCLSYELTVEQMLDWAEMAGIESVLISRDTTIHKLKHELKWNEALYRLQK

SEQ ID NO: 42 - DNA, 1491 bp, artificial sequence
  Codon-optimized coding region of B. subtilis       AI.
atgttgcaaactaaggattacgaattctggttcgttactggttctcaacacttgtacggtgaagaaactttggaattggtcgatcaacacgctaagtctatctgtgaaggtttgtccggtgtctcttccagatacaagatcacccacaagccagttgtcacctcttccgaaactatcagacaattgttgagagaagctgaatactctgaaacttgtgctggtatcatcacctggatgcacactttctctccagctaagatgtggatcgaaggtttgtcttcctaccaaaagccattgatgcacttgcacacccaatacaacagagacatcccttggggtactatcgacatggatttcatgaactctaaccaatccgctcacggtgacagagaatacggttacatcaactccagaatgggtttgtccagaaaggttgtcgctggttactgggacgatgaagaagtcaagaaggaaatctctcaatggatggacaccgctgctgctttgaacgaatccagacacatcaaggttgctagattcggtgacaacatgagacacgttgctgtcactgacggtgacaaggttggtgctcacatccaattcggttggcaagttgacggttacggtatcggtgacttggttgaagtcatgaacagaatcaccgacgatgaagttgacactttgtacgctgaatacgatagattgtacgtcatctctgaagaaaccaagagagacgaagctaaggttgcttccatcaaggaacaagctaagatcgaattgggtttgaccactttcttggaacaaggtggttactctgctttcaccacttccttcgaagtcttgcacggtatgaagcaattgccaggtttggctgttcaaagattgatggaaaagggttacggtttcgctggtgaaggtgactggaagaccgctgctttggtcagaatgatgaagatcatgtctcaaggtaaaagaacctccttcatggaagactacacttaccacttcgaaccaggtaacgaaatgatcttgggttctcacatgttggaagtttgtccaactgtcgctttggaccaaccaaagatcgaagttcacccattgtctatcggtggtaaagaagatccagctagattcgtcttcaacggtatctctggttccgctatccaagcctctttggttgacatcggtggtagattcagattggttttgaacgaagtcaacggtcaagaaatcgaaaaggacatgccaaacttgccagttgctagagtcttgtggaagccagaaccatctttgaagactgctgctgaagcctggatcttggctggtggtgctcaccacacctgtttgtcttacgaattgactgtcgaacaaatgttggactgggctgaaatggctggtatcgaatctgttttgatctccagagataccactatccacaagttgaagcacgaattgaagtggaacgaagccttgtacagattgcaaaagtaa

SEQ ID NO: 43 - DNA, 16404 bp, artificial sequence
  constructed plasmid
aaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattaggcgcctacttctagggggcctatcaagtaaattactcctggtacactgaagtatataagggatatagaagcaaatagttgtcagtgcaatccttcaagacgattgggaaaatactgtaatataaatcgtaaaggaaaattggaaattttttaaagatgtcttcactggttactcttaataacggtctgaaaatgcccctagtcggcttagggtgctggaaaattgacaaaaaagtctgtgcgaatcaaatttatgaagctatcaaattaggctaccgtttattcgatggtgcttgcgactacggcaacgaaaaggaagttggtgaaggtatcaggaaagccatctccgaaggtcttgtttctagaaaggatatatttgttgtttcaaagttatggaacaattttcaccatcctgatcatgtaaaattagctttaaagaagaccttaagcgatatgggacttgattatttagacctgtattatattcacttcccaatcgccttcaaatatgttccatttgaagagaaataccctccaggattctatacgggcgcagaaggattctatacgggcgcagaactagtgatctcgaggttccagagctcggatccaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcatcccgggttagatgagagtcttttccagttcgcttaaggggacaatcttggaattatagcgatcccaattttcattatccacatcggatatgctttccattacatgccatggaaaattgtcattcagaaatttatcaaaaggaactgcaattttattagagtcatataacaatgaccacatggccttataacaaccaccaagggcacatgagtttggtgtttctagcctaaaattaccctttgtagcaccaatgacttgagcaaacttcttcacaatagcatcgtttttagaagccccacctacaaaaaaagtcctttctggccttttatttaggtagtcccgcagcggagattcatcgtaatcaaacttcacgattgtatcttcgttcagtctctgttgtgagcttgcgtttgaatccgaaagcaggggagatattcttaccctgcaacttaaagcctgtgattctacaatatttttggcatcgtgcctcttgtctttgaacttggccacctctctttcaatcatacccgtttttggattgaagataacccttttgtttatggcttttacgctaggaacgatctcccccagaggaaaatatacacctaattcattttcactactttctgagtcatctagcacagcttgattaaaaagagtccaatcgttagtcttctcataattattttcccgttctttgtttaactcgtctcttatcctctcccttgccaaagaaccattacaataacaaatcatacccatataatggtttggcagagttggatgaatgaaaagatgatagttcggagaggggtgatacttatcggtgaccagaagaactgtagtacttgttcctagggaaacgagaacgtcattcttccgcaggggtaaagaacatatagtggctaaattatccccagtcatgggagagaccttgcagtttgtattgaaaccgtacttctcaataaaatatttacagatggtacccgctatcaaatttttcatgggtgctctcattaatttttgtctgatagttttatccttagaagaactatcaattagatgtagtagctcatcactgaattttctttcacgtatatcataaaggttcataccacaggcatctgcctcctctaattcaacaagatggcccactaagatagaagtcaaaaaattagacactaaagaaatggtctttgttttttcgtaagcttctggttctaattgtgcaattttcagaatttgaggaccagtaaatctaaaatgggctctggaccctgttaattgagccattttttcaggcccacctatgcactcttcaaactcttgacattgctttgcagtactgtggtcttgccaattgggggcggtttgccttgcaaatgctacagagctcacgtagtgcaataaatctttttccggtttcttattcaattgctctaacagagattcggcttgggaggaccagtagacagacccgtgctgctggcaggaccctgagacggccataactttgttcaatggaaatttagcctcgcgatatttcgagagaaccagatctagagcctctaaccacatggctacgggacattcgatagtgtcgccgtgtatatagacacccttctttgtgtgataatgcggaagatccttttcaaattccactgtttctgaatggacaatttttaggtcctggttaatggcgagacatttcagttgttgggtcgaaagatcaaacccaagatagtatgagtctaaagacattgtgttggaaacctctcttgtctgtctctgaattactgaacacaacatactagtcgtacggttttattttttacttatattgctggtagggtaaaaaaatataactcctaggaataggttgtctatatgtttttgtcttgcttctataattgtaacaaacaaggaaagggaaaatactgggtgtaaaagccattgagtcaagttaggtcatcccttttatacaaaatttttcaattttttttccaagattcttgtacgattaattattttttttttgcgtcctacagcgtgatgaaaatttccgcctgctgcaagatgagcgggaacgggcgaaatgtgcacgcgcacaacttacgaaacgcggatgagtcactgacagccaccgcagaggttctgactcctactgagctctattggaggtggcagaaccggtaccggaggagaccgctataaccggtttgaatttattgtcacagtgtcacatcagcggcaactcagaagtttgacagcaagcaagttcatcattcgaactagccttattgttttagttcagtgacagcgaactgccgtactcgatgctttatttctcacggtagagcggaagaacagataggggcagcgtgagaagagttagaaagtaaatttttatcacgtctgaagtattcttattcataggaaattttgcaaggttttttagctcaataacgggctaagttatataaggtgttcacgcgattttcttgttatgtatacctcttctggcgcgcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacacgtacgactagtatgactcaattcactgacattgataagttggccgtctccaccataagaattttggctgtggacaccgtatccaaggccaactcaggtcacccaggtgctccattgggtatggcaccagctgcacacgttctatggagtcaaatgcgcatgaacccaaccaacccagactggatcaacagagatagatttgtcttgtctaacggtcacgcggtcgctttgttgtattctatgctacatttgactggttacgatctgtctattgaagacttgaaacagttcagacagttgggttccagaacaccaggtcatcctgaatttgagttgccaggtgttgaagttactaccggtccattaggtcaaggtatctccaacgctgttggtatggccatggctcaagctaacctggctgccacttacaacaagccgggctttaccttgtctgacaactacacctatgttttcttgggtgacggttgtttgcaagaaggtatttcttcagaagcttcctccttggctggtcatttgaaattgggtaacttgattgccatctacgatgacaacaagatcactatcgatggtgctaccagtatctcattcgatgaagatgttgctaagagatacgaagcctacggttgggaagttttgtacgtagaaaatggtaacgaagatctagccggtattgccaaggctattgctcaagctaagttatccaaggacaaaccaactttgatcaaaatgaccacaaccattggttacggttccttgcatgccggctctcactctgtgcacggtgccccattgaaagcagatgatgttaaacaactaaagagcaaattcggtttcaacccagacaagtcctttgttgttccacaagaagtttacgaccactaccaaaagacaattttaaagccaggtgtcgaagccaacaacaagtggaacaagttgttcagcgaataccaaaagaaattcccagaattaggtgctgaattggctagaagattgagcggccaactacccgcaaattgggaatctaagttgccaacttacaccgccaaggactctgccgtggccactagaaaattatcagaaactgttcttgaggatgtttacaatcaattgccagagttgattggtggttctgccgatttaacaccttctaacttgaccagatggaaggaagcccttgacttccaacctccttcttccggttcaggtaactactctggtagatacattaggtacggtattagagaacacgctatgggtgccataatgaacggtatttcagctttcggtgccaactacaaaccatacggtggtactttcttgaacttcgtttcttatgctgctggtgccgttagattgtccgctttgtctggccacccagttatttgggttgctacacatgactctatcggtgtcggtgaagatggtccaacacatcaacctattgaaactttagcacacttcagatccctaccaaacattcaagtttggagaccagctgatggtaacgaagtttctgccgcctacaagaactctttagaatccaagcatactccaagtatcattgctttgtccagacaaaacttgccacaattggaaggtagctctattgaaagcgcttctaagggtggttacgtactacaagatgttgctaacccagatattattttagtggctactggttccgaagtgtctttgagtgttgaagctgctaagactttggccgcaaagaacatcaaggctcgtgttgtttctctaccagatttcttcacttttgacaaacaacccctagaatacagactatcagtcttaccagacaacgttccaatcatgtctgttgaagttttggctaccacatgttggggcaaatacgctcatcaatccttcggtattgacagatttggtgcctccggtaaggcaccagaagtcttcaagttcttcggtttcaccccagaaggtgttgctgaaagagctcaaaagaccattgcattctataagggtgacaagctaatttctcctttgaaaaaagctttctaaattctgatcgtagatcatcagatttgatatgatattatttgtgaaaaaatgaaataaaactttatacaacttaaatacaactttttttataaacgattaagcaaaaaaatagtttcaaacttttaacaatattccaaacactcagtccttttccttcttatattataggtgtacgtattatagaaaaatttcaatgattactttttctttctttttccttgtaccagcacatggccgagcttgaatgttaaacccttcgagagaatcacaccattcaagtataaagccaataaagaatataactcctaaaaggctaattgaaaccctgtgatttttgcccgggtttaaggcgcgccctttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaccactagcatggctgccggtgtcccaaaaattgatgcgttagaatctttgggcaatcctttggaggatgccaagagagctgcagcatacagagcagttgatgaaaatttaaaatttgatgatcacaaaattattggaattggtagtggtagcacagtggtttatgttgccgaaagaattggacaatatttgcatgaccctaaattttatgaagtagcgtctaaattcatttgcattccaacaggattccaatcaagaaacttgattttggataacaagttgcaattaggctccattgaacagtatcctcgcattgatatagcgtttgacggtgctgatgaagtggatgagaatttacaattaattaaaggtggtggtgcttgtctatttcaagaaaaattggttagtactagtgctaaaaccttcattgtcgttgctgattcaagaaaaaagtcaccaaaacatttaggtaagaactggaggcaaggtgttcccattgaaattgtaccttcctcatacgtgagggtcaagaatgatctattagaacaattgcatgctgaaaaagttgacatcagacaaggaggttctgctaaagcaggtcctgttgtaactgacaataataacttcattatcgatgcggatttcggtgaaatttccgatccaagaaaattgcatagagaaatcaaactgttagtgggcgtggtggaaacaggtttattcatcgacaacgcttcaaaagcctacttcggtaattctgacggtagtgttgaagttaccgaaaagtgagcggccgcgtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagatgcagtaatatacacagattcccgcggacgtgggaaggaaaaaattagataacaaaatctgagtgatatggaaattccgctgtatagctcatatctttccctccaccgcggtggtcgactttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaacttaagactcgagatggtcaaaccaattatagctcccagtatccttgcttctgacttcgccaacttgggttgcgaatgtcataaggtcatcaacgccggcgcagattggttacatatcgatgtcatggacggccattttgttccaaacattactctgggccaaccaattgttacctccctacgtcgttctgtgccacgccctggcgatgctagcaacacagaaaagaagcccactgcgttcttcgattgtcacatgatggttgaaaatcctgaaaaatgggtcgacgattttgctaaatgtggtgctgaccaatttacgttccactacgaggccacacaagaccctttgcatttagttaagttgattaagtctaagggcatcaaagctgcatgcgccatcaaacctggtacttctgttgacgttttatttgaactagctcctcatttggatatggctcttgttatgactgtggaacctgggtttggaggccaaaaattcatggaagacatgatgccaaaagtggaaactttgagagccaagttcccccatttgaatatccaagtcgatggtggtttgggcaaggagaccatcccgaaagccgccaaagccggtgccaacgttattgtcgctggtaccagtgttttcactgcagctgacccgcacgatgttatctccttcatgaaagaagaagtctcgaaggaattgcgttctagagatttgctagattagacgtctgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacgtacgactagtatgtctgaaccagctcaaaagaaacaaaaggttgctaacaactctctagaacaattgaaagcctccggcactgtcgttgttgccgacactggtgatttcggctctattgccaagtttcaacctcaagactccacaactaacccatcattgatcttggctgctgccaagcaaccaacttacgccaagttgatcgatgttgccgtggaatacggtaagaagcatggtaagaccaccgaagaacaagtcgaaaatgctgtggacagattgttagtcgaattcggtaaggagatcttaaagattgttccaggcagagtctccaccgaagttgatgctagattgtcttttgacactcaagctaccattgaaaaggctagacatatcattaaattgtttgaacaagaaggtgtctccaaggaaagagtccttattaaaattgcttccacttgggaaggtattcaagctgccaaagaattggaagaaaaggacggtatccactgtaatttgactctattattctccttcgttcaagcagttgcctgtgccgaggcccaagttactttgatttccccatttgttggtagaattctagactggtacaaatccagcactggtaaagattacaagggtgaagccgacccaggtgttatttccgtcaagaaaatctacaactactacaagaagtacggttacaagactattgttatgggtgcttctttcagaagcactgacgaaatcaaaaacttggctggtgttgactatctaacaatttctccagctttattggacaagttgatgaacagtactgaacctttcccaagagttttggaccctgtctccgctaagaaggaagccggcgacaagatttcttacatcagcgacgaatctaaattcagattcgacttgaatgaagacgctatggccactgaaaaattgtccgaaggtatcagaaaattctctgccgatattgttactctattcgacttgattgaaaagaaagttaccgcttaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcggatccacgatcgcattgcggattacgtattctaatgttcagtaccgttcgtataatgtatgctatacgaagttatgcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtacccgccaactctgttcgagaatgatgtaatcaagaaggtctcacaaaaccatccaggcagtaccacttcccaagtattgcttagatgggcaactcagagaggcattgccgtcattccaaaatcttccaagaaggaaaggttacttggcaacctagaaatcgaaaaaaagttcactttaacggagcaagaattgaaggatatttctgcactaaatgccaacatcagatttaatgatccatggacctggttggatggtaaattccccacttttgcctgatccagccagtaaaatccatactcaacgacgatatgaacaaatttccctcattccgatgctgtatatgtgtataaatttttacatgctcttctgtttagacacagaacagctttaaataaaatgttggatatactttttctgcctgtggtgtcatccacgcttttaattcatctcttgtatggttgacaatttggctattttttaacagaacccaacggtaattgaaattaaaagggaaacgagtgggggcgatgagtgagtgatacggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 44 - protein, 440 aa, artificial sequence
  constructed xylose isomerase
MAKEYFPQIQKIQYQGPKSTDPLSFKYYNPEEVINGKTMREHLKFALSWWHTMGGDGTDMFGCGTTDKTWGQSDPAARAKAKVDAAFEIMDKLSIDYYCFHDRDLSPEYGSLKATNDQLDIVTDYIKEKQGDKFKCLWGTAKCFDHPRFMHGAGTSPSADVFAFSAAQIKKALESTVKLGANGYVFWGGREGYETLLNTNMGLELDNMARLMKMAVEYGRSIGFKGDFYIEPKPKEPTKHQYDFDTATVLGFLRKYGLDKDFKMNIEANHATLAQHTFQHELRVARDNGVFGSIDANQGDVLLGWDTDQFPTNIYDTTMCMYEVIKAGGFTNGGLNFDAKARRGSFTPEDIFYSYIAGMDAFALGFRAALKLIEDGRIDKFVADRYASWNTGIGADIIAGKADFASLEKYALEKGEVTASLSSGRQEMLESIVNNVLFSL

SEQ ID NO: 45 - DNA, 1323 bp, artificial sequence
  codon optimized coding region for constructed       xylose isomerase VDxylA
atggctaaggaatacttcccacaaatccaaaagatccaataccaaggtccaaagtccactgacccattgtccttcaagtactacaacccagaagaagtcatcaacggtaaaaccatgagagaacacttgaagttcgctttgtcttggtggcacaccatgggtggtgacggtactgatatgttcggttgtggtactactgacaagacttggggtcaatctgatccagctgctagagctaaggctaaggttgacgctgctttcgaaatcatggacaagttgtccatcgattactactgtttccacgacagagatttgtctccagaatacggttccttgaaggctaccaacgaccaattggatatcgtcactgactacatcaaggaaaagcaaggtgacaagttcaagtgtttgtggggtactgctaagtgtttcgaccacccaagattcatgcacggtgctggtacttctccatccgctgacgttttcgctttctctgctgctcaaatcaagaaggctttggaatccaccgttaagttgggtgctaacggttacgtcttctggggtggtagagaaggttacgaaaccttgttgaacactaacatgggtttggaattggacaacatggctagattgatgaagatggctgttgaatacggtagatctatcggtttcaagggtgacttctacatcgaaccaaagccaaaggaaccaactaagcaccaatacgacttcgataccgctactgtcttgggtttcttgagaaagtacggtttggacaaggatttcaagatgaacatcgaagctaaccacgctaccttggctcaacacactttccaacacgaattgagagttgctagagacaacggtgtcttcggttccatcgatgctaaccaaggtgacgttttgttgggttgggacaccgatcaattcccaactaacatctacgacaccactatgtgtatgtacgaagtcatcaaggctggtggtttcaccaacggtggtttgaacttcgacgctaaggctagaagaggttctttcactccagaagacatcttctactcctacatcgctggtatggacgctttcgctttgggtttcagagctgctttgaagttgatcgaagacggtagaatcgataagttcgttgctgacagatacgcttcttggaacaccggtatcggtgctgacatcatcgctggtaaagctgatttcgcttctttggaaaagtacgctttggaaaagggtgaagtcactgcttccttgtcctctggtagacaagaaatgttggaatccatcgtcaacaacgttttgttctctttgtga

SEQ ID NO: 46 - DNA, 2966 bp, artificial sequence
  constructed chimeric expression cassette for       VDxykA
tgacagcaggattatcgtaatacgtaatagttgaaaatctcaaaaatgtgtgggtcattacgtaaataatgataggaatgggattcttctatttttcctttttccattctgtcgaccgcacgccgaaatgcatgcaagtaacctattcaaagtaatatctcatacatgtttcatgagggtaacaacatgcgactgggtgagcatatgttccgctgatgtgatgtgcaagataaacaagcaagacagaaactaacttcttcttcatgtaataaacacaccccgcgtttatttacctatctttaaacttcaacaccttatatcataactaatatttcttgagataagcacactgcacccataccttccttaaaaacgtagcttccagtttttggtggttctggcttccttcccgattccgcccgctaaacgcataattttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccgtttttctgttagacggtgtcttgatctacttgctatcgttcaacaccaccttatcttctaactattttttttttagctcatttgaatcagcttatggtgatggcacatttttgcataaacctagctgtcctcgttgaacataggaaaaaaaaatatataaacaaggctctttcactctccttggaatcagatttgggtttgttccctttattttcatatttcttgtcatattcttttctcaattattattttctactcataacctcacgcaaaataacacagtcaaatcaatcaagtttaaacagtatggctaaggaatacttcccacaaatccaaaagatccaataccaaggtccaaagtccactgacccattgtccttcaagtactacaacccagaagaagtcatcaacggtaaaaccatgagagaacacttgaagttcgctttgtcttggtggcacaccatgggtggtgacggtactgatatgttcggttgtggtactactgacaagacttggggtcaatctgatccagctgctagagctaaggctaaggttgacgctgctttcgaaatcatggacaagttgtccatcgattactactgtttccacgacagagatttgtctccagaatacggttccttgaaggctaccaacgaccaattggatatcgtcactgactacatcaaggaaaagcaaggtgacaagttcaagtgtttgtggggtactgctaagtgtttcgaccacccaagattcatgcacggtgctggtacttctccatccgctgacgttttcgctttctctgctgctcaaatcaagaaggctttggaatccaccgttaagttgggtgctaacggttacgtcttctggggtggtagagaaggttacgaaaccttgttgaacactaacatgggtttggaattggacaacatggctagattgatgaagatggctgttgaatacggtagatctatcggtttcaagggtgacttctacatcgaaccaaagccaaaggaaccaactaagcaccaatacgacttcgataccgctactgtcttgggtttcttgagaaagtacggtttggacaaggatttcaagatgaacatcgaagctaaccacgctaccttggctcaacacactttccaacacgaattgagagttgctagagacaacggtgtcttcggttccatcgatgctaaccaaggtgacgttttgttgggttgggacaccgatcaattcccaactaacatctacgacaccactatgtgtatgtacgaagtcatcaaggctggtggtttcaccaacggtggtttgaacttcgacgctaaggctagaagaggttctttcactccagaagacatcttctactcctacatcgctggtatggacgctttcgctttgggtttcagagctgctttgaagttgatcgaagacggtagaatcgataagttcgttgctgacagatacgcttcttggaacaccggtatcggtgctgacatcatcgctggtaaagctgatttcgcttctttggaaaagtacgctttggaaaagggtgaagtcactgcttccttgtcctctggtagacaagaaatgttggaatccatcgtcaacaacgttttgttctctttgtgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgag

SEQ ID NO: 47 - DNA, 8601 bp, artificial sequence
  constructed plasmid
aaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggcgccacttgtgcatgattaccgacaaccaaaaccagtcacagattctattaatcctccaaatgtaaacataaccacctccacaaccaacaagaacctagatggcatttatattttgccagctcctcgtatgaatcccccggctcaaacacaataccaaatgattcatgcgccagacagcatgcaacatccaccaacatttagtaaaaacaacacatcaagcaatcctaaatcccaccaatactcaaagtagaagatcagcatcctttcaattgctgaaaggttcacctaaagtaccgctcatattccaaaaggattcttcactacatagaaagggcagccaattgtgtgtttttcagaaagggttttaaaaaaacaggagggtgcttgttcttgttgttccctaccatcgatggatttcgaaaaactatttataggaccatctgattttcacctccatcattgtatcatatactaacaagcatatccaaatttgtaattctatcatgaaatttccagagaaagaaacgcaagggaactgagaaatcaaacactagttgacagcaggattatcgtaatacgtaatagttgaaaatctcaaaaatgtgtgggtcattacgtaaataatgataggaatgggattcttctatttttcctttttccattctgtcgaccgcacgccgaaatgcatgcaagtaacctattcaaagtaatatctcatacatgtttcatgagggtaacaacatgcgactgggtgagcatatgttccgctgatgtgatgtgcaagataaacaagcaagacagaaactaacttcttcttcatgtaataaacacaccccgcgtttatttacctatctttaaacttcaacaccttatatcataactaatatttcttgagataagcacactgcacccataccttccttaaaaacgtagcttccagtttttggtggttctggcttccttcccgattccgcccgctaaacgcataattttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccgtttttctgttagacggtgtcttgatctacttgctatcgttcaacaccaccttatcttctaactattttttttttagctcatttgaatcagcttatggtgatggcacatttttgcataaacctagctgtcctcgttgaacataggaaaaaaaaatatataaacaaggctctttcactctccttggaatcagatttgggtttgttccctttattttcatatttcttgtcatattcttttctcaattattattttctactcataacctcacgcaaaataacacagtcaaatcaatcaagtttaaacagtatggctaaggaatacttcccacaaatccaaaagatccaataccaaggtccaaagtccactgacccattgtccttcaagtactacaacccagaagaagtcatcaacggtaaaaccatgagagaacacttgaagttcgctttgtcttggtggcacaccatgggtggtgacggtactgatatgttcggttgtggtactactgacaagacttggggtcaatctgatccagctgctagagctaaggctaaggttgacgctgctttcgaaatcatggacaagttgtccatcgattactactgtttccacgacagagatttgtctccagaatacggttccttgaaggctaccaacgaccaattggatatcgtcactgactacatcaaggaaaagcaaggtgacaagttcaagtgtttgtggggtactgctaagtgtttcgaccacccaagattcatgcacggtgctggtacttctccatccgctgacgttttcgctttctctgctgctcaaatcaagaaggctttggaatccaccgttaagttgggtgctaacggttacgtcttctggggtggtagagaaggttacgaaaccttgttgaacactaacatgggtttggaattggacaacatggctagattgatgaagatggctgttgaatacggtagatctatcggtttcaagggtgacttctacatcgaaccaaagccaaaggaaccaactaagcaccaatacgacttcgataccgctactgtcttgggtttcttgagaaagtacggtttggacaaggatttcaagatgaacatcgaagctaaccacgctaccttggctcaacacactttccaacacgaattgagagttgctagagacaacggtgtcttcggttccatcgatgctaaccaaggtgacgttttgttgggttgggacaccgatcaattcccaactaacatctacgacaccactatgtgtatgtacgaagtcatcaaggctggtggtttcaccaacggtggtttgaacttcgacgctaaggctagaagaggttctttcactccagaagacatcttctactcctacatcgctggtatggacgctttcgctttgggtttcagagctgctttgaagttgatcgaagacggtagaatcgataagttcgttgctgacagatacgcttcttggaacaccggtatcggtgctgacatcatcgctggtaaagctgatttcgcttctttggaaaagtacgctttggaaaagggtgaagtcactgcttccttgtcctctggtagacaagaaatgttggaatccatcgtcaacaacgttttgttctctttgtgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcccgggatttaaatgtggcttactccattgttgatgcaaaagttgtaaatttcacgaattatttaatgcgttccttgcaaccttctattttgatgaacatcaggaattgaaacaaaaaaaaggcttcaatctcaacggaaaacgggaagaaaactacactcgattatactatatatgccaagaagattctccgacagattgtctacttaatttcacataatatatcttgttttactagcttattatatagcgtcgcatttaattcatggcgccatcacccgcagggaatataacgacaaggccgataccacgggaaaaatagggcgagcggaaatactaaaagaaaaataagcttccgaaataaaacaccgacaatgaagttcttggcaaggttcggttaggatccgcattgcggattacgtattctaatgttcagtaccgttcgtataatgtatgctatacgaagttatgcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccgagatacccacttcgaaagttactgatattataactcttgtgtcctctcttctaataccttactttcacctttctcacgtagttaaagttgcaacaacacattttgtcctcatccaatttcttctatagaatatccgtttgcctccaggagtgaagaaatgatagcagtaacactgtgaaagcgagactaagagaaacgacttaaagctcgaagacttcttgaggatacgtttatgtttctgtggcttcttcttcgcggcgcggttctcgcgtataggaatgttctaagacaagaaggcatgaagttatgttaacagattctatatctactcgctacgcatatataaacggattcatcattgaaacaatggtacttgtggtaatgtgtacgacgatttcaacccgaataaaagcaaaagtgcaaaaaaaaaacaagaagcgcttagcactgttgaatcatttagaacactactaatgctggtaatacggcgccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 48 - DNA, 8645 bp, artificial sequence
  constructed plasmid
accttggctcaacacactttccaacacgaattgagagttgctagagacaacggtgtcttcggttccatcgatgctaaccaaggtgacgttttgttgggttgggacaccgatcaattcccaactaacatctacgacaccactatgtgtatgtacgaagtcatcaaggctggtggtttcaccaacggtggtttgaacttcgacgctaaggctagaagaggttctttcactccagaagacatcttctactcctacatcgctggtatggacgctttcgctttgggtttcagagctgctttgaagttgatcgaagacggtagaatcgataagttcgttgctgacagatacgcttcttggaacaccggtatcggtgctgacatcatcgctggtaaagctgatttcgcttctttggaaaagtacgctttggaaaagggtgaagtcactgcttccttgtcctctggtagacaagaaatgttggaatccatcgtcaacaacgttttgttctctttgtgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcccgggatttaaatccttataccctcatcttaccgcagtgcggttttacgcctcatgttatttttgccagctttaataacaacatcagtaatattacgttattggatcttccactccggttcgaggaaaaaaagagaggaggagaaacgcataagctacaataatgagtgagttaacgctttaatttgctcagtgatcattgctagccggatatttgtgtttttgtagaacccagccatacctaatcatctcagtatattatctgaattcttgactcaaaatatggattttctcgaacgtctcacttccaatcatcccatcaaatgctgtggatgaaaacatttaaaacacgggcaagaaaaaatgagtatagtatagttgtggccagttcttgttgtactaatgcacttgctttcgtatcaaagttgaagaagcactgaaagaacaacaaaaaatttattaaaagcaaaaatcattctacgttcaacgaaaatgtaaggcaaatatatctttatataggcaagcataaccgacgcggatccgcattgcggattacgtattctaatgttcagtaccgttcgtataatgtatgctatacgaagttatgcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtacctgaaaggtttacgatattagctgctaagttctcccttgccctagtgaattacgtttttttcgattaaaagggttggtcccgaaattgacctcttatatgtacctcaattgcagatagcatcaaatttagactacgtcatcaaccaaattacaccctatgaaaaacataagattataagcatgcgtttgtgtaaaggtcataccattttatacgttgacgcaatggtgcaaatagaaaggtttacgaaagctcttattccgcttagcgcaattctattgttaatttcaagtaaaaagataaatatctcacgtccttatattatctttgattaaaatttcacgatcaatgtaacgaaaaaaggtcgcaaatatttctttttcatcactctgattaaacaatggcatcatgaaacgctcattggatcgttacacgttctcattgtacatatgtatgatttagaacagcttgttcgcctgggcgccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggcgcccgaggtttgagtcaatgtacgagattttgaaaccatgagggatgtgccctcaaactttatgatctggtttctttttttatttatatcgtttttatgctgggttacgttgttgttttatattccttgtctagtattacggtcagcagaggccaaatcctatatgcattatatgtggttagcattgactccttaatagaacggccagttatcaatcaaattgcctataagcatggatcactagcggctagtattgtattgcatttggaatagccctcttcgttgagccacaaagatatcttgcggaaatgtggctaaagcaacttttgatgattgctgggaaacggtttggtaaactgctgaagcaattgttgtacacaatacttctgcttttggccccatctggatttttcatttccgcttttgcctcaggtgcaacaataactatctttgagcatcgtaccaccaactagttgacagcaggattatcgtaatacgtaatagttgaaaatctcaaaaatgtgtgggtcattacgtaaataatgataggaatgggattcttctatttttcctttttccattctgtcgaccgcacgccgaaatgcatgcaagtaacctattcaaagtaatatctcatacatgtttcatgagggtaacaacatgcgactgggtgagcatatgttccgctgatgtgatgtgcaagataaacaagcaagacagaaactaacttcttcttcatgtaataaacacaccccgcgtttatttacctatctttaaacttcaacaccttatatcataactaatatttcttgagataagcacactgcacccataccttccttaaaaacgtagcttccagtttttggtggttctggcttccttcccgattccgcccgctaaacgcataattttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccgtttttctgttagacggtgtcttgatctacttgctatcgttcaacaccaccttatcttctaactattttttttttagctcatttgaatcagcttatggtgatggcacatttttgcataaacctagctgtcctcgttgaacataggaaaaaaaaatatataaacaaggctctttcactctccttggaatcagatttgggtttgttccctttattttcatatttcttgtcatattcttttctcaattattattttctactcataacctcacgcaaaataacacagtcaaatcaatcaagtttaaacagtatggctaaggaatacttcccacaaatccaaaagatccaataccaaggtccaaagtccactgacccattgtccttcaagtactacaacccagaagaagtcatcaacggtaaaaccatgagagaacacttgaagttcgctttgtcttggtggcacaccatgggtggtgacggtactgatatgttcggttgtggtactactgacaagacttggggtcaatctgatccagctgctagagctaaggctaaggttgacgctgctttcgaaatcatggacaagttgtccatcgattactactgtttccacgacagagatttgtctccagaatacggttccttgaaggctaccaacgaccaattggatatcgtcactgactacatcaaggaaaagcaaggtgacaagttcaagtgtttgtggggtactgctaagtgtttcgaccacccaagattcatgcacggtgctggtacttctccatccgctgacgttttcgctttctctgctgctcaaatcaagaaggctttggaatccaccgttaagttgggtgctaacggttacgtcttctggggtggtagagaaggttacgaaaccttgttgaacactaacatgggtttggaattggacaacatggctagattgatgaagatggctgttgaatacggtagatctatcggtttcaagggtgacttctacatcgaaccaaagccaaaggaaccaactaagcaccaatacgacttcgataccgctactgtcttgggtttcttgagaaagtacggtttggacaaggatttcaagatgaacatcgaagctaaccacgct

SEQ ID NO: 49 - DNA, 8537 bp, artificial sequence
  constructed plasmid
aaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggcgccttgagcattagttgatcattaccgctgcagtcagttaatgatatccctaaataggcatattgtacaatttcaaatatattacccgaattacaagaacaatagggtcttctacaatattactatgaacaattcataggggaacatagtccacttcaatgtcggtgccaacgattctatttgttgtaagtatatatgctgtttcccggcaatactttatattgggggaattttctggaggtttaagggcatattactacgtcaaggctgggattgaccttctggtatatattacgttgcgcaggacattccttatacttgagcctttgtaaaaacttcttcgttctcctgaccaaccgttcaattatgttgtgaattttttgatccgagtttgaaacatccaaattcacaaacaaaccgttgtcatcctgagttggcgtactagttgacagcaggattatcgtaatacgtaatagttgaaaatctcaaaaatgtgtgggtcattacgtaaataatgataggaatgggattcttctatttttcctttttccattctgtcgaccgcacgccgaaatgcatgcaagtaacctattcaaagtaatatctcatacatgtttcatgagggtaacaacatgcgactgggtgagcatatgttccgctgatgtgatgtgcaagataaacaagcaagacagaaactaacttcttcttcatgtaataaacacaccccgcgtttatttacctatctttaaacttcaacaccttatatcataactaatatttcttgagataagcacactgcacccataccttccttaaaaacgtagcttccagtttttggtggttctggcttccttcccgattccgcccgctaaacgcataattttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccgtttttctgttagacggtgtcttgatctacttgctatcgttcaacaccaccttatcttctaactattttttttttagctcatttgaatcagcttatggtgatggcacatttttgcataaacctagctgtcctcgttgaacataggaaaaaaaaatatataaacaaggctctttcactctccttggaatcagatttgggtttgttccctttattttcatatttcttgtcatattcttttctcaattattattttctactcataacctcacgcaaaataacacagtcaaatcaatcaagtttaaacagtatggctaaggaatacttcccacaaatccaaaagatccaataccaaggtccaaagtccactgacccattgtccttcaagtactacaacccagaagaagtcatcaacggtaaaaccatgagagaacacttgaagttcgctttgtcttggtggcacaccatgggtggtgacggtactgatatgttcggttgtggtactactgacaagacttggggtcaatctgatccagctgctagagctaaggctaaggttgacgctgctttcgaaatcatggacaagttgtccatcgattactactgtttccacgacagagatttgtctccagaatacggttccttgaaggctaccaacgaccaattggatatcgtcactgactacatcaaggaaaagcaaggtgacaagttcaagtgtttgtggggtactgctaagtgtttcgaccacccaagattcatgcacggtgctggtacttctccatccgctgacgttttcgctttctctgctgctcaaatcaagaaggctttggaatccaccgttaagttgggtgctaacggttacgtcttctggggtggtagagaaggttacgaaaccttgttgaacactaacatgggtttggaattggacaacatggctagattgatgaagatggctgttgaatacggtagatctatcggtttcaagggtgacttctacatcgaaccaaagccaaaggaaccaactaagcaccaatacgacttcgataccgctactgtcttgggtttcttgagaaagtacggtttggacaaggatttcaagatgaacatcgaagctaaccacgctaccttggctcaacacactttccaacacgaattgagagttgctagagacaacggtgtcttcggttccatcgatgctaaccaaggtgacgttttgttgggttgggacaccgatcaattcccaactaacatctacgacaccactatgtgtatgtacgaagtcatcaaggctggtggtttcaccaacggtggtttgaacttcgacgctaaggctagaagaggttctttcactccagaagacatcttctactcctacatcgctggtatggacgctttcgctttgggtttcagagctgctttgaagttgatcgaagacggtagaatcgataagttcgttgctgacagatacgcttcttggaacaccggtatcggtgctgacatcatcgctggtaaagctgatttcgcttctttggaaaagtacgctttggaaaagggtgaagtcactgcttccttgtcctctggtagacaagaaatgttggaatccatcgtcaacaacgttttgttctctttgtgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcccgggatttaaatgccgttgacattcatgtaagcagtattagcttttaaactgccattaacgcacctatacgattgcggcggtgcgaaaacagtaactagcactcttacggctcattgtttcctctatccaaagttgcctatatcacaataaaattagaaattaatcattttaataagcttgctgccattcagtgactcgcaatctacgttttaaacgtaatttaaaaatccacttccacctacatattttgcaacagtcgtacgacagaaactgaggctctataaaatgagatgtgctaattgttctttcttggctggtttcagctacatctttctgtaatcaatctacaaatttacacgcgagcttcattttgacagtaaacaaatgtaaaagacacatgcaataaaagcggtccagaaaacaaacgacaaagccaccaaaaatgtttgcaaacgttggatttagaactttgagggggatccgcattgcggattacgtattctaatgttcagtaccgttcgtataatgtatgctatacgaagttatgcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccaattgtttctccacatgtgtaccgtataaagttcttgttatgaattagaaggattatcacaaaagccctccatcaaatctgataatttcattacccaaagactgtaataccttgggtgtaaagatcgtattttttgatgtcttcgtgatattttgcagatctttccattttataaatttctgcaaatatcgtgcatcagctcatcttggaattggcacaatctgcgtaatttttttcttggttctcttctccttttgattttaagagcgctttctattcagtagcagattttcagtaaataaagccatctttctcaagtaaactcatgatcatgcagttgttcaaaattacaccaataacgatgttatctttaaaactttccccagaaagaagatggcacttttgcactaaatacttcaaactatttaactttgaagcaggacatattgccggggcgccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 50 - DNA, 6728 bp, artificial sequence
  constructed vector
acatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaatgtccaatttactgcccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtgttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggat

SEQ ID NO: 51 - DNA, 2431 bp, artificial sequence
  chimeric expression cassette for BSaraA
cttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatgttgcaaactaaggattacgaattctggttcgttactggttctcaacacttgtacggtgaagaaactttggaattggtcgatcaacacgctaagtctatctgtgaaggtttgtccggtgtctcttccagatacaagatcacccacaagccagttgtcacctcttccgaaactatcagacaattgttgagagaagctgaatactctgaaacttgtgctggtatcatcacctggatgcacactttctctccagctaagatgtggatcgaaggtttgtcttcctaccaaaagccattgatgcacttgcacacccaatacaacagagacatcccttggggtactatcgacatggatttcatgaactctaaccaatccgctcacggtgacagagaatacggttacatcaactccagaatgggtttgtccagaaaggttgtcgctggttactgggacgatgaagaagtcaagaaggaaatctctcaatggatggacaccgctgctgctttgaacgaatccagacacatcaaggttgctagattcggtgacaacatgagacacgttgctgtcactgacggtgacaaggttggtgctcacatccaattcggttggcaagttgacggttacggtatcggtgacttggttgaagtcatgaacagaatcaccgacgatgaagttgacactttgtacgctgaatacgatagattgtacgtcatctctgaagaaaccaagagagacgaagctaaggttgcttccatcaaggaacaagctaagatcgaattgggtttgaccactttcttggaacaaggtggttactctgctttcaccacttccttcgaagtcttgcacggtatgaagcaattgccaggtttggctgttcaaagattgatggaaaagggttacggtttcgctggtgaaggtgactggaagaccgctgctttggtcagaatgatgaagatcatgtctcaaggtaaaagaacctccttcatggaagactacacttaccacttcgaaccaggtaacgaaatgatcttgggttctcacatgttggaagtttgtccaactgtcgctttggaccaaccaaagatcgaagttcacccattgtctatcggtggtaaagaagatccagctagattcgtcttcaacggtatctctggttccgctatccaagcctctttggttgacatcggtggtagattcagattggttttgaacgaagtcaacggtcaagaaatcgaaaaggacatgccaaacttgccagttgctagagtcttgtggaagccagaaccatctttgaagactgctgctgaagcctggatcttggctggtggtgctcaccacacctgtttgtcttacgaattgactgtcgaacaaatgttggactgggctgaaatggctggtatcgaatctgttttgatctccagagataccactatccacaagttgaagcacgaattgaagtggaacacgaagccttgtacagattgcaaaagtaattaattaatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcgg

SEQ ID NO: 52 - protein, 566 aa, Escherichia coli
MAIAIGLDFGSDSVRALAVDCASGEEIATSVEWYPRWQKGQFCDAPNNQFRHHPRDYIESMEAALKTVLAELSVEQRAAVVGIGVDSTGSTPAPIDADGNVLALRPEFAENPNAMFVLWKDHTAVERSEEITRLCHAPGNVDYSRYIGGIYSSEWFWAKILHVTRQDSAVAQSAASWIELCDWVPALLSGTTRPQDIRRGRCSAGHKSLWHESWGGLPPASFFDELDPILNRHLPSPLFTDTWTADIPVGTLCPEWAQRLGLPESVVISGGAFDCHMGAVGAGAQPNALVKVIGTSTCDILIADKQSVGERAVKGICGQVDGSVVPGFIGLEAGQSAFGDIYAWFGRVLSWPLEQLAAQHPELKAQINASQKQLLPALTEAWAKNPSLDHLPVVLDWFNGRRSPNANQRLKGVITDLNLATDAPLLFGGLIAATAFGARAIMECFTDQGIAVNNVMALGGIARKNQVIMQACCDVLNRPLQIVASDQCCALGAAIFAAVAAKVHADIPSAQQKMASAVEKTLQPRSEQAQRFEQLYRRYQQWAMSAEQHYLPTSAPAQAAQAVATL

SEQ ID NO: 53 - DNA, 1701 bp, artificial sequence
  codon optimized coding region for E. coli       ribulokinase
atggctatcgctatcggtttggacttcggttctgactccgttagagctttggctgttgactgtgcttccggtgaagaaatcgctacttctgtcgaatggtatccaagatggcaaaagggtcaattctgtgacgctccaaacaaccaattcagacaccacccaagagattacatcgaatctatggaagctgctttgaagactgttttggctgaattgtctgtcgaacaaagagctgctgttgtcggtatcggtgttgactctactggttccaccccagctccaatcgacgctgatggtaacgttttggctttgagaccagaattcgctgaaaacccaaacgctatgttcgttttgtggaaggaccacactgctgtcgaaagatccgaagaaatcaccagattgtgtcacgctccaggtaacgttgactactccagatacatcggtggtatctactcttccgaatggttctgggctaagattttgcacgttactagacaagactctgctgtcgctcaatctgctgcttcctggatcgaattgtgtgactgggttccagctttgttgtctggtactactagaccacaagatatcagaagaggtagatgttctgctggtcacaagtccttgtggcacgaatcttggggtggtttgccaccagcttccttcttcgacgaattggacccaatcttgaacagacacttgccatctccattgttcaccgacacttggaccgctgatatcccagtcggtactttgtgtccagaatgggctcaaagattgggtttgccagaatctgttgtcatctccggtggtgctttcgactgtcacatgggtgctgttggtgctggtgctcaaccaaacgctttggttaaggtcatcggtacttccacctgtgacatcttgatcgctgataagcaatctgttggtgaaagagctgtcaagggtatctgtggtcaagttgacggttccgttgtcccaggtttcatcggtttggaagctggtcaatctgctttcggtgacatctacgcttggttcggtagagttttgtcctggccattggaacaattggctgctcaacacccagaattgaaggctcaaatcaacgcttctcaaaagcaattgttgccagctttgactgaagcctgggctaagaacccatccttggaccacttgccagttgtcttggattggttcaacggtagaagatccccaaacgctaaccaaagattgaagggtgttatcactgacttgaacttggctaccgatgctccattgttgttcggtggtttgatcgctgctactgctttcggtgctagagctatcatggaatgtttcaccgaccaaggtatcgctgttaacaacgtcatggctttgggtggtatcgctagaaagaaccaagttatcatgcaagcctgttgtgacgtcttgaacagaccattgcaaatcgttgcttctgatcaatgttgtgctttgggtgctgctatcttcgctgctgttgctgctaaggtccacgctgacatcccatccgctcaacaaaagatggcttctgctgtcgaaaagaccttgcaaccaagatccgaacaagctcaaagattcgaacaattgtacagaagataccaacaatgggctatgtccgctgaacaacactacttgccaacttctgctccagctcaagctgctcaagctgttgctaccttgtaa

SEQ ID NO: 54 - DNA, 2911 bp, artificial sequence
  constructed chimeric expression cassette for       ECaraB
gagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcgaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaataaaacatggctatcgctatcggtttggacttcggttctgactccgttagagctttggctgttgactgtgcttccggtgaagaaatcgctacttctgtcgaatggtatccaagatggcaaaagggtcaattctgtgacgctccaaacaaccaattcagacaccacccaagagattacatcgaatctatggaagctgctttgaagactgttttggctgaattgtctgtcgaacaaagagctgctgttgtcggtatcggtgttgactctactggttccaccccagctccaatcgacgctgatggtaacgttttggctttgagaccagaattcgctgaaaacccaaacgctatgttcgttttgtggaaggaccacactgctgtcgaaagatccgaagaaatcaccagattgtgtcacgctccaggtaacgttgactactccagatacatcggtggtatctactcttccgaatggttctgggctaagattttgcacgttactagacaagactctgctgtcgctcaatctgctgcttcctggatcgaattgtgtgactgggttccagctttgttgtctggtactactagaccacaagatatcagaagaggtagatgttctgctggtcacaagtccttgtggcacgaatcttggggtggtttgccaccagcttccttcttcgacgaattggacccaatcttgaacagacacttgccatctccattgttcaccgacacttggaccgctgatatcccagtcggtactttgtgtccagaatgggctcaaagattgggtttgccagaatctgttgtcatctccggtggtgctttcgactgtcacatgggtgctgttggtgctggtgctcaaccaaacgctttggttaaggtcatcggtacttccacctgtgacatcttgatcgctgataagcaatctgttggtgaaagagctgtcaagggtatctgtggtcaagttgacggttccgttgtcccaggtttcatcggtttggaagctggtcaatctgctttcggtgacatctacgcttggttcggtagagttttgtcctggccattggaacaattggctgctcaacacccagaattgaaggctcaaatcaacgcttctcaaaagcaattgttgccagctttgactgaagcctgggctaagaacccatccttggaccacttgccagttgtcttggattggttcaacggtagaagatccccaaacgctaaccaaagattgaagggtgttatcactgacttgaacttggctaccgatgctccattgttgttcggtggtttgatcgctgctactgctttcggtgctagagctatcatggaatgtttcaccgaccaaggtatcgctgttaacaacgtcatggctttgggtggtatcgctagaaagaaccaagttatcatgcaagcctgttgtgacgtcttgaacagaccattgcaaatcgttgcttctgatcaatgttgtgctttgggtgctgctatcttcgctgctgttgctgctaaggtccacgctgacatcccatccgctcaacaaaagatggcttctgctgtcgaaaagaccttgcaaccaagatccgaacaagctcaaagattcgaacaattgtacagaagataccaacaatgggctatgtccgctgaacaacactacttgccaacttctbamhgctccagctcaagctgctcaagctgttgctaccttgtaaggatccaggagcaatgcaaaatctaggggtagaattactttttgaaaaggaaaaatattcaggtttgttgtttttatgtaagttgtatgatttgatatacatatatatatatatataatatatattgtacatgtgtttttccggggaagaatggattatccggaggtgtgaataaaatgatgacgattataggtttgtgttgtaatatttagataactcaattctcgccagtttgaactccaacctagactggttcaaagcttttgctatcaagatgagatatatggaattttcgtctttatcgtccacttgtatctttatttcctcgtcatcttcatcaatattgattccattaataatcgatttatcgctcagagtgttgaccaattcggtcttgttggggaagaaatgttccatttttcttcccaagttttgaattctttcacaaacccaggcaattctttgtaagcctaatgcagcagaagaaccctttaaaaaatggccca

SEQ ID NO: 55 - protein, 231 aa, Escherichia coli
MLEDLKRQVLEANLALPKHNLVTLTWGNVSAVDRERGVFVIKPSGVDYSIMTADDMVVVSIETGEVVEGAKKPSSDTPTHRLLYQAFPSIGGIVHTHSRHATIWAQAGQSIPATGTTHADYFYGTIPCTRKMTDAEINGEYEWETGNVIVETFEKQGIDAAQMPGVLVHSHGPFAWGKNAEDAVHNAIVLEEVAYMGIFCRQLAPQLPDMQQTLLNKHYLRKHGAKAYYGQ

SEQ ID NO: 56 - DNA, 696 bp, artificial sequence
  E coli L-ribulose-5-phosphate 4-epimerase codon      optimized coding region
atgttggaagatttgaagagacaagttttggaagctaacttggctttgccaaagcacaacttggtcaccttgacctggggtaacgtctctgctgttgacagagaaagaggtgtcttcgttatcaagccatctggtgttgattactccatcatgactgctgacgatatggttgtcgtttccatcgaaaccggtgaagtcgttgaaggtgctaagaagccatcttccgacaccccaactcacagattgttgtaccaagccttcccatctatcggtggtatcgtccacactcactccagacacgctaccatctgggctcaagctggtcaatctatcccagctactggtactactcacgctgactacttctacggtactatcccatgtactagaaagatgaccgatgctgaaatcaacggtgaatacgaatgggaaactggtaacgtcatcgttgaaaccttcgaaaagcaaggtatcgacgctgctcaaatgccaggtgtcttggttcactctcacggtccattcgcttggggtaaaaacgctgaagatgctgttcacaacgctatcgtcttggaagaagttgcttacatgggtatcttctgtagacaattggctccacaattgccagacatgcaacaaaccttgttgaacaagcactacttgagaaagcacggtgctaaggcttactacggtcaataa

SEQ ID NO: 57 - DNA, 1691 bp, artificial sequence
  constructed chimeric expression cassette for       ECaraD
agttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgttggaagatttgaagagacaagttttggaagctaacttggctttgccaaagcacaacttggtcaccttgacctggggtaacgtctctgctgttgacagagaaagaggtgtcttcgttatcaagccatctggtgttgattactccatcatgactgctgacgatatggttgtcgtttccatcgaaaccggtgaagtcgttgaaggtgctaagaagccatcttccgacaccccaactcacagattgttgtaccaagccttcccatctatcggtggtatcgtccacactcactccagacacgctaccatctgggctcaagctggtcaatctatcccagctactggtactactcacgctgactacttctacggtactatcccatgtactagaaagatgaccgatgctgaaatcaacggtgaatacgaatgggaaactggtaacgtcatcgttgaaaccttcgaaaagcaaggtatcgacgctgctcaaatgccaggtgtcttggttcactctcacggtccattcgcttggggtaaaaacgctgaagatgctgttcacaacgctatcgtcttggaagaagttgcttacatgggtatcttctgtagacaattggctccacaattgccagacatgcaacaaaccttgttgaacaagcactacttgagaaagcacggtgctaaggcttactacggtcaataagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggt

SEQ ID NO: 58 - DNA, 8202 bp, artificial sequence
  constructed plasmid
  misc_feature (421)..(421): n is a, c, g, or t
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttntgcgggcggccgctggacaatttattcatggcatcgtcattgatataagtggcttgagctgtggataagaaaagccatatatttatataaacatttagatatgaataggaagtagattgttcgacgcaactacccgttcaagaagtataatggggaatggtctcatcttccctcacaggatatagttctctgaagagatacatacgtttgtgtatactatgcttctttatcaactcaagttttgtagaggaagacgttgaagatggtgatgtgacatctttactattctccagcacgttttcagtatttacttaatcgtatattaatgacgtcccttatctattaactttccggtttttctttttttcggtgaatgttctttccgttttagtgaatttttcaattgtaattgacgcaatcggtttataacaagcagacataaatatcaagctcgagccaaatcacaaaaaaagccttatagbamhcttgccctgacaaagaatatacaactcgggaaggatccgagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcgaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaataaaacatggctatcgctatcggtttggacttcggttctgactccgttagagctttggctgttgactgtgcttccggtgaagaaatcgctacttctgtcgaatggtatccaagatggcaaaagggtcaattctgtgacgctccaaacaaccaattcagacaccacccaagagattacatcgaatctatggaagctgctttgaagactgttttggctgaattgtctgtcgaacaaagagctgctgttgtcggtatcggtgttgactctactggttccaccccagctccaatcgacgctgatggtaacgttttggctttgagaccagaattcgctgaaaacccaaacgctatgttcgttttgtggaaggaccacactgctgtcgaaagatccgaagaaatcaccagattgtgtcacgctccaggtaacgttgactactccagatacatcggtggtatctactcttccgaatggttctgggctaagattttgcacgttactagacaagactctgctgtcgctcaatctgctgcttcctggatcgaattgtgtgactgggttccagctttgttgtctggtactactagaccacaagatatcagaagaggtagatgttctgctggtcacaagtccttgtggcacgaatcttggggtggtttgccaccagcttccttcttcgacgaattggacccaatcttgaacagacacttgccatctccattgttcaccgacacttggaccgctgatatcccagtcggtactttgtgtccagaatgggctcaaagattgggtttgccagaatctgttgtcatctccggtggtgctttcgactgtcacatgggtgctgttggtgctggtgctcaaccaaacgctttggttaaggtcatcggtacttccacctgtgacatcttgatcgctgataagcaatctgttggtgaaagagctgtcaagggtatctgtggtcaagttgacggttccgttgtcccaggtttcatcggtttggaagctggtcaatctgctttcggtgacatctacgcttggttcggtagagttttgtcctggccattggaacaattggctgctcaacacccagaattgaaggctcaaatcaacgcttctcaaaagcaattgttgccagctttgactgaagcctgggctaagaacccatccttggaccacttgccagttgtcttggattggttcaacggtagaagatccccaaacgctaaccaaagattgaagggtgttatcactgacttgaacttggctaccgatgctccattgttgttcggtggtttgatcgctgctactgctttcggtgctagagctatcatggaatgtttcaccgaccaaggtatcgctgttaacaacgtcatggctttgggtggtatcgctagaaagaaccaagttatcatgcaagcctgttgtgacgtcttgaacagaccattgcaaatcgttgcttctgatcaatgttgtgctttgggtgctgctatcttcgctgctgttgctgctaaggtccacgctgacatcccatccgctcaacaaaagatggcttctgctgtcgaaaagaccttgcaaccaagatccgaacaagctcaaagattcgaacaattgtacagaagataccaacaatgggctatgtccgctgaacaacactacttgccaacttctbamhgctccagctcaagctgctcaagctgttgctaccttgtaaggatccaggagcaatgcaaaatctaggggtagaattactttttgaaaaggaaaaatattcaggtttgttgtttttatgtaagttgtatgatttgatatacatatatatatatatataatatatattgtacatgtgtttttccggggaagaatggattatccggaggtgtgaataaaatgatgacgattataggtttgtgttgtaatatttagataactcaattctcgccagtttgaactccaacctagactggttcaaagcttttgctatcaagatgagatatatggaattttcgtctttatcgtccacttgtatctttatttcctcgtcatcttcatcaatattgattccattaataatcgatttatcgctcagagtgttgaccaattcggtcttgttggggaagaaatgttccatttttcttcccaagttttgaattctttcacaaacccaggcaattctttgtaagcctaatgcagcagaagaaccctttaaaaaatggcccattaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggccttctaaatcaccatttttggtgaacggccttgataaggatgttagttgtgttattgctgggttagacacgaaggtaaattaccaccgtttggctgttacactgcagtatttgcagaaggattctgttcactttgttggtacaaatgttgattctactttcccgcaaaagggttatacatttcccggtgcaggctccatgattgaatcattggcattctcatctaataggaggccatcgtactgtggtaagccaaatcaaaatatgctaaacagcattatatcggcattcaacctggatagatcaaagtgctgtatggttggtgacagattaaacaccgatatgaaattcggtgttgaaggtgggttaggtggcacactactcgttttgagtggtattgaaaccgaagagagagccttgaagatttcgcacgattatccaagacctaaattttacattgataaacttggtgaasccatctacaccttaaccaataatgagttatagggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattccctaataagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 59 - DNA, 10887 bp, artificial sequence
  constructed plasmid
ggcctcgagcggaccggatccttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatgttgcaaactaaggattacgaattctggttcgttactggttctcaacacttgtacggtgaagaaactttggaattggtcgatcaacacgctaagtctatctgtgaaggtttgtccggtgtctcttccagatacaagatcacccacaagccagttgtcacctcttccgaaactatcagacaattgttgagagaagctgaatactctgaaacttgtgctggtatcatcacctggatgcacactttctctccagctaagatgtggatcgaaggtttgtcttcctaccaaaagccattgatgcacttgcacacccaatacaacagagacatcccttggggtactatcgacatggatttcatgaactctaaccaatccgctcacggtgacagagaatacggttacatcaactccagaatgggtttgtccagaaaggttgtcgctggttactgggacgatgaagaagtcaagaaggaaatctctcaatggatggacaccgctgctgctttgaacgaatccagacacatcaaggttgctagattcggtgacaacatgagacacgttgctgtcactgacggtgacaaggttggtgctcacatccaattcggttggcaagttgacggttacggtatcggtgacttggttgaagtcatgaacagaatcaccgacgatgaagttgacactttgtacgctgaatacgatagattgtacgtcatctctgaagaaaccaagagagacgaagctaaggttgcttccatcaaggaacaagctaagatcgaattgggtttgaccactttcttggaacaaggtggttactctgctttcaccacttccttcgaagtcttgcacggtatgaagcaattgccaggtttggctgttcaaagattgatggaaaagggttacggtttcgctggtgaaggtgactggaagaccgctgctttggtcagaatgatgaagatcatgtctcaaggtaaaagaacctccttcatggaagactacacttaccacttcgaaccaggtaacgaaatgatcttgggttctcacatgttggaagtttgtccaactgtcgctttggaccaaccaaagatcgaagttcacccattgtctatcggtggtaaagaagatccagctagattcgtcttcaacggtatctctggttccgctatccaagcctctttggttgacatcggtggtagattcagattggttttgaacgaagtcaacggtcaagaaatcgaaaaggacatgccaaacttgccagttgctagagtcttgtggaagccagaaccatctttgaagactgctgctgaagcctggatcttggctggtggtgctcaccacacctgtttgtcttacgaattgactgtcgaacaaatgttggactgggctgaaatggctggtatcgaatctgttttgatctccagagataccactatccacaagttgaagcacgaattgaagtggaacgaagccttgtacagattgcaaaagtaattaattaatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcgggcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttattgaccgtagtaagccttagcaccgtgctttctcaagtagtgcttgttcaacaaggtttgttgcatgtctggcaattgtggagccaattgtctacagaagatacccatgtaagcaacttcttccaagacgatagcgttgtgaacagcatcttcagcgtttttaccccaagcgaatggaccgtgagagtgaaccaagacacctggcatttgagcagcgtcgataccttgcttttcgaaggtttcaacgatgacgttaccagtttcccattcgtattcaccgttgatttcagcatcggtcatctttctagtacatgggatagtaccgtagaagtagtcagcgtgagtagtaccagtagctgggatagattgaccagcttgagcccagatggtagcgtgtctggagtgagtgtggacgataccaccgatagatgggaaggcttggtacaacaatctgtgagttggggtgtcggaagatggcttcttagcaccttcaacgacttcaccggtttcgatggaaacgacaaccatatcgtcagcagtcatgatggagtaatcaacaccagatggcttgataacgaagacacctctttctctgtcaacagcagagacgttaccccaggtcaaggtgaccaagttgtgctttggcaaagccaagttagcttccaaaacttgtctcttcaaatcttccaacattttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactagatctatccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatctcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagtcaccggtggc

SEQ ID NO: 60 - protein, 500 aa, Escherichia coli
MTIFDNYEVWFVIGSQHLYGPETLRQVTQHAEHVVNALNTEAKLPCKLVLKPLGTTPDEITAICRDANYDDPCAGLVVWLHTFSPAKMWINGLTMLNKPLLQFHTQFNAALPWDSIDMDFMNLNQTAHGGREFGFIGARMRQQHAVVTGHWQDKQAHERIGSWMRQAVSKQDTRHLKVCRFGDNMREVAVTDGDKVAAQIKFGFSVNTWAVGDLVQVVNSISDGDVNALVDEYESCYTMTPATQIHGEKRQNVLEAARIELGMKRFLEQGGFHAFTTTFEDLHGLKQLPGLAVQRLMQQGYGFAGEGDWKTAALLRIMKVMSTGLQGGTSFMEDYTYHFEKGNDLVLGSHMLEVCPSIAVEEKPILDVQHLGIGGKDDPARLIFNTQTGPAIVASLIDLGDRYRLLVNCIDTVKTPHSLPKLPVANALWKAQPDLPTASEAWILAGGAHHTVFSHALNLNDMRQFAEMHDIEITVIDNDTRLPAFKDALRWNEVYYGFRR

SEQ ID NO: 61 - protein, 493 aa, Bacillus licheniformis
MIQAKTHVFWFVTGSQHLYGEEAVQEVEEHSKMICNGLNDGDLRFQVEYKAVATSLDGVRKLFEEANRDEECAGIITWMHTFSPAKMWIPGLSELNKPLLHFHTQFNRDIPWDKIDMDFMNINQSAHGDREYGFIGARLGIPRKVIAGYWEDREVKRSIDKWMSAAVAYIESRHIKVARFGDNMRNVAVTEGDKIEAQIQLGWSVDGYGIGDLVTEINAVSEQSLSELISEYEELYEWPEGEAARESVKEQARIELGLKRFLSSGGYTAFTTTFEDLHGMKQLPGLAVQRLMAEGYGFGGEGDWKTAALVRMMKMMAGGKETSFMEDYTYHFEPGNEMILGSHMLEVCPSIAEHKPRIEVHPLSMGAKDDPARLVFDGIAGPAVNVSLIDLGGRFRLVINKVEAVKVPHDMPNLPVARVLWKPQPSLRTSAEAWILAGGAHHTCLSYQLTAEQMLDWAEMSGIEAVLINRDTTILNLRNELKWSEAAYRLRKF

SEQ ID NO: 62 - protein, 488 aa, Clostridium acetobutylicum
MLENKKMEFWFVVGSQHLYGEEALKEVRKNSETIVDELNKSANLPYKIIFKDLATSADKIKEIMKEVNYRDEVAGVITWMHTFSPAKMWIAGTKILQKPLLHFATQYNENIPWKTIDMDYMNLHQSAHGDREYGFINARLKKHNKVVVGYWKDKEVQKQVSDWMKVAAGYIASESIKVARFGDNMRNVAVTEGDKVEAQIQFGWTVDYFGIGDLVAEMDKVSQDEINKTYEEFKDLYILDPGENDPAFYEKQVKEQIKIEIGLRRFLEKGNYNAFTTNFEDLYGMKQLPGLAVQRLNAEGYGFAGEGDWKTAALDRLLKVMTNNTATGFMEDYTYELSRGNEKALGAHMLEVDPTFASDKPKVIVKPLGIGDKEDPARLIFNGSTGKGVAVSMLDLGTHYRLIINGLTAVKPDEDMPNLPVAKMVWKPEPNFIEGVKSWIYAGGGHHTVVSLELTVEQVYDWSRMVGLEAVIIDKDTKLRDIIEKTTK

SEQ ID NO: 63 - protein, 474 aa, Leuconostoc mesenteroides
MADIKDYKFWFVTGSQFLYGPEVLKQVEEDSKKIIEKLNESGNLPYPIEFKTVGVTAENITEAMKEANYDDSVAGVITWAHTFSPAKNWIRGTQLLNKPLLHLATQMLNNIPYDSIDFDYMNLNQSAHGDREYAFINARLRLNNKIVFGHWADEAVQVQIGKWMDVAVAYEESFKIKVVTFADKMRNVAVTDGDKIEAQIKFGWTVDYWGVGDLVTYVNAIDDADIDNLYIELQDKYDFVAGQNDSEKYEHNVKYQLREYLGIKRFLTDKGYSAFTTNFEDLVGLEQLPGLAAQLLMADGFGFAGEGDWKTAALTRLLKIVSHNQATAFMEDYTLDLRQGHEAILGSHMLEVDPTIASDKPRVEVHPLGIGGKEDPARLVFSGRTGDAVDVTISDFGDEFKLISYDVTGNKPEAETPYLPVAKQLWTPKAGLKAGAEGWLTVGGGHHTTLSFSVDSEQLTDLANLFGVTYVDIK

SEQ ID NO: 64 - protein, 474 aa, Lactobacillus plantarum
MLSVPDYEFWFVTGSQHLYGEEQLKSVAKDAQDIADKLNASGKLPYKVVFKDVMTTAESITNFMKEVNYNDKVAGVITWMHTFSPAKNWIRGTELLQKPLLHLATQYLNNIPYADIDFDYMNLNQSAHGDREYAYINARLQKHNKIVYGYWGDEDVQEQIARWEDVAVAYNESFKVKVARFGDTMRNVAVTEGDKVEAQIKMGWTVDYYGIGDLVEEINKVSDADVDKEYADLESRYEMVQGDNDADTYKHSVRVQLAQYLGIKRFLERGGYTAFTTNFEDLWGMEQLPGLASQLLIRDGYGFGAEGDWKTAALGRVMKIMSHNKQTAFMEDYTLDLRHGHEAILGSHMLEVDPSIASDKPRVEVHPLDIGGKDDPARLVFTGSEGEAIDVTVADFRDGFKMISYAVDANKPEAETPNLPVAKQLWTPKMGLKKGALEWMQAGGGHHTMLSFSLTEEQMEDYATMVGMTKAFLK

SEQ ID NO: 65 - protein, 474 aa, Pediococcus pentosaceus
MKKVQDYEFWFVTGSQFLYGEETLRSVEKDAKEIVDKLNESKKLPYPVKFKLVATTAENITEVMKEVNYNDKVAGVITWMHTFSPAKNWIRGTELLQKPLLHLATQFLNHIPYDTIDFDYMNLNQSAHGDREYAFINARLRKNNKIISGYWGDEGIQKQIAKWMDVAVAYNESYGIKVVTFADKMRNVAVTDGDKIEAQIKFGWTVDYWGVADLVEEVNAVSDEDIDKKYEEMKNDYNFVEGQNSSEKFEHNTKYQIREYFGLKKFMDDRGYTAFTTNFEDLAGLEQLPGLAAQMLMAEGYGFAGEGDWKTAALDRLLKIMAHNKQTVFMEDYTLDLREGHEAILGSHMLEVDPSIASDTPRVEVHPLDIGGKEDPARFVFTGMEGDAVDVTMADYGDEFKLMSYDVTGNKTEKETPYLPVAKQLWTPKQGWKQGAEGWLTLGGGHHTVLSFAIDAEQLQDLSNMFGLTYVNIK

SEQ ID NO: 66 - protein, 33 aa, artificial sequence
  237-269 amino acid motif with variation at       multiple positions
  VARIANT (2)..(2): R or K
  VARIANT (6)..(6): R or K
  VARIANT (11)..(11): I or M
  VARIANT (12)..(12): E or K
  VARIANT (14)..(14): I or M
  VARIANT (15)..(15): L or M
  VARIANT (16)..(16): V or T or D
  VARIANT (17)..(17): R or A
  VARIANT (18)..(18): E or N
  VARIANT (20)..(20): A or C
  VARIANT (21)..(21): K or N or R
  VARIANT (24)..(24): S or V or T
  VARIANT (28)..(28): E or Q
  VARIANT (31)..(31): H or I or Y
IXYQAXEEIAXXKXXXXXGXXAFXNTFXDLXGM

SEQ ID NO: 67 - protein, 33 aa, artificial sequence; identical to SEQ ID NO: 66
  Amino acid sequence of the 237-269 Motif shown       in FIG. 2A
  MISC_FEATURE (2)..(2): Arg or Lys
  MISC_FEATURE (6)..(6): Arg or Lys
  MISC_FEATURE (11)..(11): Ile or Met
  MISC_FEATURE (12)..(12): any amino acid
  MISC_FEATURE (14)..(14): Ile or Met
  MISC_FEATURE (15)..(15): Leu or Met
  MISC_FEATURE (16)..(16): any amino acid
  MISC_FEATURE (17)..(17): Arg or Ala
  MISC_FEATURE (18)..(18): Glu or Asn
  MISC_FEATURE (20)..(20): Ala or Cys
  MISC_FEATURE (21)..(21): any amino acid
  MISC_FEATURE (24)..(24): any amino acid
  MISC_FEATURE (28)..(28): Gln or Glu
  MISC_FEATURE (31)..(31): any amino acid
  MISC_FEATURE (32)..(32): any amino acid

SEQ ID NO: 68 - protein, 477 aa, Ruminococcus flavefaciens
MKFWFVTGSQFLYGEETLRQVEEDSKKIVDGLDLPFPVEYKMTVKKESEIERIIKEANYDDECAGIITFCHTFSPSKMWINGLALLQKPWLHFHTQFNETIPNEGIDMDYMNLHQSAHGDREHGFIGARLRMPRAVVAGHWKDKKVQEKIAEWQRAAVGALFSKSLKIVRFGDNMREVAVTEGDKIEAQLKLGWQVNTFAVGDLVEIMNAVKDAEIDELMKEYAELYDYDKADEETIRYQAREEIAIEKILVREGAKAFSNTFEDLHGMRQLPGLATQHLMHKGYGFGAEGDWKTAGMTAIVKAMYPEGNTSFMEDYTYDYKHELILGSHMLEVCPSIAADKPRIEVHKLGIGGKEAPARIVFEGRAGSAKALSLIDIGGRFRLISQDVECEKPFQSMPNLPVARTMWKPAPSFLEGLECWIIAGGAHHTVLSYDITDETVRDFARIMGIELVVINKDTTKEKLERDIMIGDMIYGR

SEQ ID NO: 69 - protein, 477 aa, Ruminococcus flavefaciens
MKFWFITGSQFLYGEETLRQVDEDSKKIVAGLKLPFPVEYKSTVKTESEIQRIIKEANFDDECAGVITFCHTFSPSKMWINGLALLQKPWLHFHTQFNETIPNEAIDMDYMNLHQSAHGDREHGFIGARLRMPRAVVAGHWQDPEVQAKIAEWQRAAVGVMFSKSLKIVRFGDNMREVAVTEGDKIEAQLKLGWQVNTFAVGDLVEIMNAVTDAEIDALMKEYAELYDYKKEDEETIRYQAREEIAIEKILVREGAKAYSNTFEDLHGMKQLPGLATQHLMHKGYGFGAEGDWKTAGMTAIVKAMYPEGNTSFMEDYTYDYKQELILGSHMLEVCPSIAADRPRIEVHKLGIGGKEPPARIVFEGKAGSAKVLSLIDIGGRLRLIQQDIECVKPFQSMPNLPVARTMWRPAPSFLDGLECWIIAGGAHHTVLSYDISDEAVRSFARIMGIELVVINKDTTVNGLERDIMIGDVIYGR

SEQ ID NO: 70 - protein, 477 aa, Ruminococcus flavefaciens
MKFWFITGSQFLYGEETLRQVDEDSKKIVAGLKLPFPVEYKSTVKTEREIERIIKEANYDDECAGIITFCHTFSPSKMWINGLALLQKPWLHFHTQFNETIPNEAIDMDYMNLHQSAHGDREHGFIGARLRMPRAVVAGHWQDPEVQAKIAEWQRAAVGVMFSKSLKIVRFGDNMREVAVTEGDKVEAQLKLGWQVNTFAVGDLVEIMNAVTNTEIDALMKEYAELYDYKKEDEETIRYQAREEIAIEKILVREGAKAFSNTFEDLHGMKQLPGLATQHLMHKGYGFGAEGDWKTAGMTAIVKAMYPDGNTSFMEDYTYDYKQQLILGSHMLEVCPSIAADKPRIEVHKLGIGGKEPPARIVFEGKAGSAKALSLIDIGGRLRLISQDVECVKPFQSMPNLPVARTMWRPAPSFLEGLECWIVAGGAHHTVLSYDISDEAVRSFARIMGIELVVINKDTTVNGLERDIMIGDVIYGR

SEQ ID NO: 71 - protein, 477 aa, Ruminococcus flavefaciens
MKFWFVTGSQFLYGEETLRQVEEDSKKIVAGLKLPFPVEYKLTVKKEAEITKIIKEANYDDECAGIITFCHTFSPSKMWINGLRSLQKPWLHFHTQFNDNIPNDAIDMDYMNLHQSAHGDREHGFIGARLRMPRAVVAGHWADPAVQEKIADWMRAAVGVQFSKSLKIVRFGDNMREVAVTEGDKIEAQIKLGWQVNTFAVGDLVQIMNAVTDAEIDALMKEYAELYDFDKADEECIRYQAREEIAIEKILVREGAMAFSNTFEDLHGMKQLPGLATQHLMHKGYGFGAEGDWKTAGMTAIIKAMYPDGNTSFMEDYNYDYKHELILGAHMLEVCPSIAAGRPRIEVHPLGIGGKDAPARIVFEGKAGSAKAISLIDIGGRLRLIAQDVECEKPFQTMPNLPVARTMWKPAPSFLEGLECWIIAGGAHHTVLSYDISDETVRDFARIMGIELVVINKNTNKYQLERDMMIGDVIYGR